(19) United States
(12) Patent Application Publication
(10) Pub. No.: US 2024/0150797 A1
(43) Pub. Date: May 9, 2024

(54) BIOCATALYTICAL PRODUCTION OF DIHYDROCHALCONES

(71) Applicant: Symrise AG, Holzminden (DE)

(72) Inventors: Torsten Geissler, Einbeck (DE); Jakob Peter Ley, Holzminden (DE); Bastian Zirpel, Holzminden (DE); Dong Yi, Greifswald (DE); Uwe Bornscheuer, Greifswald (DE)

(73) Assignee: Symrise AG, Holzminden (DE)

(21) Appl. No.: 18/278,159

(22) PCT Filed: Mar. 3, 2021
(86) PCT No.: PCT/EP2021/055319

§ 371 Date: Aug. 21, 2023

(51) Int. Cl. C12P 7/26; C12N 9/02; C12N 9/90; C12N 15/63; C12R 1/19

(52) U.S. Cl. CPC C12P 7/26; C12N 9/0004; C12N 9/90; C12N 15/63; C12R 2001/19; C12Y 505/01006

(57) ABSTRACT

The present invention lies in the field of food ingredients and concerns a method for the production of dihydrochalcones from various educts as well as the corresponding enzymes, which are used for the production of dihydrochalcones. Furthermore, the present invention concerns transgenic microorganisms and vectors for expressing the enzymes according to the invention.

The present invention lies in the field of food ingredients and concerns a method for the production of dihydrochalcones from various educts as well as the corresponding enzymes, which are used for the production of dihydrochalcones. Furthermore, the present invention concerns transgenic microorganisms and vectors for expressing the enzymes according to the invention.

There is a constant need of flavouring substances in the food technology area. Especially the class of dihydrochalcones is of interest, as these substances or mixtures of these substances exhibit superior properties compared to other flavouring substances. The natural source of dihydrochalcones are plants, an especially high content can be found in apple leaves (Adamu et al., Investigations on the formation of dihydrochalcones in apple (Malus sp.) leaves. Acta Horticulturae 2019, 1242, 415-420). As the recovery and extraction of dihydrochalcones from plants is not favourable in terms of yield and process costs, there are several production methods for dihydrochalcones described in the literature.

A desired product is hesperetin dihydrochalcone (3).

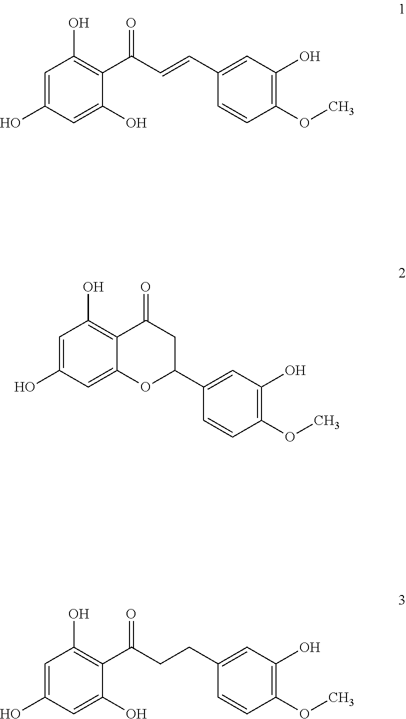 [SMILES: COc1ccc(/C=C/C(=O)c2c(O)cc(O)cc2O)cc1O ; COc1ccc(C2CC(=O)c3c(O)cc(O)cc3O2)cc1O ; COc1ccc(CCC(=O)c2c(O)cc(O)cc2O)cc1O]

The aromatic effect of hesperetin dihydrochalcone (3) as flavouring substance is described in WO 2017186299 A1. This property is also known from J. Agric. Food Chem., 25(4), 763-772 and J. Med., 1981, 24(4), 408-428. Mixtures of hesperetin dihydrochalcone (3) with corn syrup with increased fruit sugar content and other sweeteners are described in WO 2019080990 A1.

It is described that the production of hesperetin chalcone (1) from hesperetin (2) can be achieved in a one pot reaction by reacting 1,8-Diazabicyclo[5.4.0]undec-7-ene, tert-Butyldimethylsilyl chloride and hydrochloric acid (Miles, Christopher O.; et al Australian Journal of Chemistry (1989), 42(7), 1103-13).

Further methods to generate hesperetin chalcone (1) comprise the aldol condensation of trihydroxyacetone with isovanillin by addition of potassium hydroxide (Wadher, S. J.; et al International Journal of Chemical Sciences (2006), 4(4), 761-766).

In a further step, hesperetin chalcone (1) can be further reduced via hydrogenation using hydrogen or formic acid and palladium catalysts to form hesperetin dihydrochalcone (3) (Gan, Li-She; et al Bioorganic & Medicinal Chemistry Letters (2017), 27(6), 1441-1445, US20180177758 A1).

It is also described that the production of hesperetin dihydrochalcone (3) can be achieved directly from hesperetin chalcone (2) by inorganic catalysts such as iron or platinum (CN111018684).

Hesperetin dihydrochalcone (3) can also be prepared by acidic hydrolysis of neohesperidine dihydrochalcon (WO 2019080990 A1). Furthermore, hesperetin dihydrochalcone (3) can be obtained from hesperetin chalcone (2) as described in DE 2148332 A1 or CN 111018684 by dissolving hesperetin chalcone (2) in 10% aqueous KOH solution and subsequent reduction by means of hydrogen (Pd/C catalyst). The use of protective groups, other bases or reducing agents and the possibility of an acid-catalysed aldol reaction are known to the skilled person.

However, all methods described in the state of the art cannot be declared as natural production methods according to EC 1334/2008 and are limited to producing a specific dihydrochalcone.

Labelling as natural is crucial for many consumers for purchase decision, so it is clear that there is a particular need for appropriate dihydrochalcones that are allowed to carry this label. Obtaining the dihydrochalcones from plant raw materials, is a timely and costly process and for some of the dihydrochalcones listed herein not possible, as they cannot be found in nature.

Concerning enzymatic or fermentative methods only the conversion of naringenin, eriodictyol and homoeriodictyol has been reported in the state of the art (EP 2963109A1, Gall et. al Angew. Chem. Int. Ed. 2013, 52, 1-5). It is furthermore mentioned that the single enzyme flavanonol-cleaving reductase is capable of converting naringenin and homoeriodictyol to the respective dihydrochalcones (Braune et. al. 2019 Appl Environ Microbiol 85:e01233-19). None of the 4-O-methylated derivatives were converted by the enzyme.

It is furthermore known that the gut bacterium Eubacterium ramulus is able to convert naringenin-7-O-glucoside via a pathway, which involves the production of the dihydroychalcone phloretin. Unfortunately, this is further degraded to phloroglucinol and dihydrocinnamic acid (H. Schneider, M. Blaut, Arch. Microbiol. 2000, 173, 71-75).

Chalcone reduction activities to its respective dihydrochalcones are described for several organisms in literature, e.g. in Żyszka-Haberecht et al., 2018 and Stompor et al., 2016.

All of the reported methods and processes are either expensive, laborious or not available for commercial production.

A primary task of this invention was therefore to provide a method and suitable biocatalysts for the production of a variety of desired dihydrochalcones, which is preferably cost-efficient and scalable and wherein the obtained products can be labelled as “natural”.

The primary task of this invention is solved by providing a method for the biocatalytical manufacturing of dihydrochalcones, comprising or consisting of the steps:
i) providing at least one ene reductase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 24 to 46 and 169 to 176;
ii) optionally providing at least one genetically engineered chalcone isomerase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 145 to 158;
iii) providing at least one flavanone and/or at least one chalcone and/or at least one of the corresponding glycosides,
iv) incubating the at least one ene reductase provided in step i) and optionally the at least one chalcone isomerase provided in step ii) together with the at least one flavanone and/or the at least one chalcone and/or the at least one corresponding glycoside provided in step iii);
v) obtaining at least one dihydrochalcone;
vi) optionally purifying the obtained dihydrochalcone.

Dihydrochalcones are open chain flavonoids, named systematically as propanone derivatives, structurally related to 1,3-diphenylpropenones (chalcones), biosynthesized in plants and exhibiting a wide spectrum of biological activities. The conversion from chalcones to dihydrochalcones can be mediated by enzymes, which reduce the double bond of the chalcones and therefore form dihydrochalcones.

Biocatalytical manufacturing in terms of the present invention is to be understood as the manufacturing of a product from an educt under the aid of biocatalysts. Such biocatalysts are generally understood as being enzymes, which can be present as part of living biological systems (cells), partially purified or purified. Each biocatalyst catalyses a unique chemical reaction.

An enzyme used in the context of the method according to the present invention is an ene reductase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 24 to 46 and 169 to 176. Ene reductases catalyse the asymmetric reduction of electronically activated carbon-carbon double bonds with a high chemoselectivity and elevated stereoselectivity.

It was surprisingly found that the ene reductases used in the method according to the present invention showed a superior activity and selectivity for catalysing the transformation from chalcones to dihydrochalcones. The ene reductases with the SEQ ID Nos 24 to 46 and 169 to 176 were selected based on their catalytic activity and selectivity, wherein the SEQ ID Nos 24 to 46 are naturally occurring sequences from different organisms as described in the sequence description and wherein the SEQ ID Nos 169 to 176 are genetically engineered sequences derived from Arabidopsis thaliana enzyme AtDBR1.

Whenever the present disclosure refers to sequence homologies of amino acid sequences in terms of percentages, it refers to values, which can be calculated using EMBOSS Water Pairwise Sequence Alignments (Nucleotide) (http://www.ebi.ac.uk/Tools/psa/emboss_water/nucleotide.html) for nucleic acid sequences or EMBOSS Water Pairwise Sequence Alignments (Protein) (http://www.ebi.ac.uk/Tools/psa/emboss_water/) for amino acid sequences. In the case of the local sequence alignment tools provided by the European Molecular Biology Laboratory (EMBL) European Bioinformatics Institute (EBI), a modified Smith-Waterman algorithm is used (see http://www.ebi.ac.uk/Tools/psa/ and Smith, T. F. & Waterman, M. S. “Identification of common molecular subsequences” Journal of Molecular Biology, 1981 147 (1):195-197). Furthermore, here, when performing the respective pairwise alignment of two sequences using the modified Smith-Waterman algorithm, reference is made to the default parameters currently given by EMBL-EBI. These are (i) for amino acid sequences: Matrix=BLOSUM62, Gap open penalty=10 and Gap extend penalty=0.5 and (ii) for nucleic acid sequences: Matrix=DNAfull, Gap open penalty=10 and Gap extend penalty=0.5.

The term “sequence homology” can be used interchangeably with “sequence identity” in the context of the present invention. Both terms always refer to the total length of an enzyme according to the invention compared to the total length of an enzyme to which the sequence identity or sequence homology is determined.

In terms of the present invention, it is preferred to provide an additional chalcone isomerase in step ii) of the method according to the invention. Chalcone isomerases are enzymes, which catalyse the reaction from a chalcone to a flavanone and vice versa. Another name of chalcone isomerase is chalcone-flavanone isomerase.

It was surprisingly found that a genetically engineered chalcone isomerase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 145 to 158 show a superior performance in catalysing the reaction from flavanone to chalcone and can be therefore used in a method starting with a flavanone as educt for producing dihydrochalcones. It was especially surprising that chalcone isomerases as used in a method according to the invention accelerate the conversion of chalcone to dihydrochalcone by an ene reductase according to the present invention and as provided in step i) of the method according to the present invention as they are able to convert flavanone to chalcone, which is then supplied to the subsequent reaction from chalcone to dihydrochalcone.

“Genetically engineered” in terms of the present invention means that the enzyme according to the present invention is altered or modified in comparison to a naturally occurring enzyme or an enzyme known from the state of the art. Suitable modifications can be mutations in the amino acid sequence. Suitable mutagenesis methods, as well as the necessary conditions and reagents, are well known to those skilled in the art. Mutations occur at the gene level, for example, through the replacement (or substitution), removal (or deletion), or addition of bases. These mutations have different effects on the amino acid sequence of the resulting protein. In the case of substitution, so-called “nonsense” mutations can occur, causing protein biosynthesis to stop early and the resulting protein to remain dysfunctional. In the so-called “missense” mutation, only the encoded amino acid changes; these mutations result in a functional change in the resulting protein and, in the best case, may cause improved stability or activity of the resulting protein. In general nomenclature, amino acid substitution mutations are designated based on their position and the amino acid substituted, for example, as A143G. This notation means that at position 143 of the N- to C-terminal amino acid sequence, the amino acid alanine has been exchanged for guanine.

Flavanones are the first flavonoid products of the flavonoid biosynthetic pathway. They are characterized by the presence of a chiral centre at C2 and the absence of the C2-C3 bond.

Flavanones are found at high concentrations in citrus fruits. They are preferably used as educts according to the present invention and are further specified below.

Chalcones are α,β-unsaturated ketones, consisting of two aromatic rings (A and B) attached by α,β-unsaturated carbonyl system with different substituents. Chalcones preferably used as educts according to the present invention are further specified below.

The term “corresponding glycosides” in connection with the used flavanones and/or chalcones refers to corresponding flavanones and/or chalcones having a sugar bound to another functional group via a glycosidic bond. Glycosides of flavanones and/or chalcones are especially present in natural sources of flavanones and/or chalcones.

The obtained dihydrochalcones from the method according to the invention can be present as a mixture of different dihydrochalcones and/or in a mixture together with other compounds depending on the used flavanone and/or chalcone and/or their corresponding glycosides or, respectively the used starting material. They can be further purified by suitable methods known to the person skilled in the art. The obtained mixture of or purified dihydrochalcones are preferably incorporated as flavouring agents in preparations such as aroma compositions, preparations intended for nutrition or enjoyment.

Preferably, a preparation intended for nutrition or enjoyment may be selected from the group consisting of (reduced-calorie) baked goods (e.g. bread, dry biscuits, cakes, other baked articles), confectionery (e.g. muesli bar products, chocolates, chocolate bars, other products in bar form, fruit gums, dragees, hard and soft caramels, chewing gum), non-alcoholic drinks (e.g. cocoa, coffee, green tea, black tea, (green, black) tea drinks enriched with (green, black) tea extracts, rooibos tea, other herbal teas, fruit-containing soft drinks, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage preparations, spiced or marinated fresh or salt meat products), eggs or egg products (dried egg, egg white, egg yolk), cereal products (e.g. breakfast cereals, muesli bars, precooked ready-to-eat rice products), dairy products (e.g. full-fat or reduced-fat or fat-free milk drinks, rice pudding, yoghurt, kefir, cream cheese, soft cheese, hard cheese, dried milk powder, whey, butter, buttermilk, ice-cream, partially or completely hydrolysed milk-protein-containing products), products made from soy protein or other soybean fractions (e.g. soy milk and products produced therefrom, drinks containing isolated or enzymatically treated soy protein, drinks containing soy flour, preparations containing soy lecithin, fermented products such as tofu or tempeh or products produced therefrom and mixtures with fruit preparations and optionally flavours), dairy-like preparations (milk-type, yoghurt-type, dessert-type, ice cream) from protein rich plant materials (e.g. from seed materials of oat, almond, pea, lupine, lentils, faba beans, chickpea, rice, canola), plant protein-enriched non-dairy drinks, fruit preparations (e. g. jams, sorbets, fruit sauces, fruit fillings), vegetable preparations (e.g. ketchup, sauces, dried vegetables, frozen vegetables, precooked vegetables, boiled-down vegetables), snacks (e.g. baked or fried potato crisps or potato dough products, maize- or groundnut-based extrudates), fat- and oil-based products or emulsions thereof (e.g. mayonnaise, remoulade, dressings, in each case full-fat or reduced-fat), other ready-made dishes and soups (e.g. dried soups, instant soups, precooked soups), spices, spice mixtures and in particular seasonings which are used, for example, in the snacks field, sweetener preparations, tablets or sachets, other preparations for sweetening or whitening drinks.

The preparation intended for nutrition or enjoyment within the meaning of the invention can also be present as dietary supplements in the form of capsules, tablets (uncoated and coated tablets, e.g. gastro-resistant coatings), sugar-coated pills, granulates, pellets, solid mixtures, dispersions in liquid phases, as emulsions, as powders, as solutions, as pastes or as other formulations that can be swallowed or chewed.

A preferred embodiment of the present invention relates to a method according to the invention, wherein the at least one ene reductase provided in step i) is purified or partially purified. A purified ene reductase refers to an enzyme which shows a purity of 90% (w/w) or more when provided. Suitable methods for the purification of enzymes are known to the person skilled in the art. A partially purified enzyme refers to an enzyme, which has a purity of less than 90% (w/w) and is not present in a living organism.

Another preferred embodiment relates to a method according to the invention, wherein the incubation in step iv) is done for at least 5, 10, 15, 20, 25 minutes, preferably for at least 30 minutes.

Yet another embodiment relates to a method according to the invention, wherein the at least one flavanone and/or at least one chalcone and/or at least one of the corresponding glycosides provided in step iii) is selected from the group consisting of homoeriodictyol, hesperidin, hesperetin-7-glucosid, neohesperidin, naringenin, naringin, narirutin, liquiritigenin, pinocembrin, steppogenin, scuteamoenin, dihydroechiodinin, ponciretin, sakuranetin, isosakuranetin, 4,7-dihydroxy-flavanon, 4,7-dihydroxy-3′-methoxyflavanon, 3,7-dihydroxy-4′-methoxyflavanon, 3′4,7-trihydroxyflavanon, alpinentin, pinostrobin, 7-hydroxyflavanon, 4′-hydroxyflavanon, 3-hydroxyflavanon, tsugafolin.

The preferred flavanones and/or chalcones are depicted with their structural formulas as shown below.

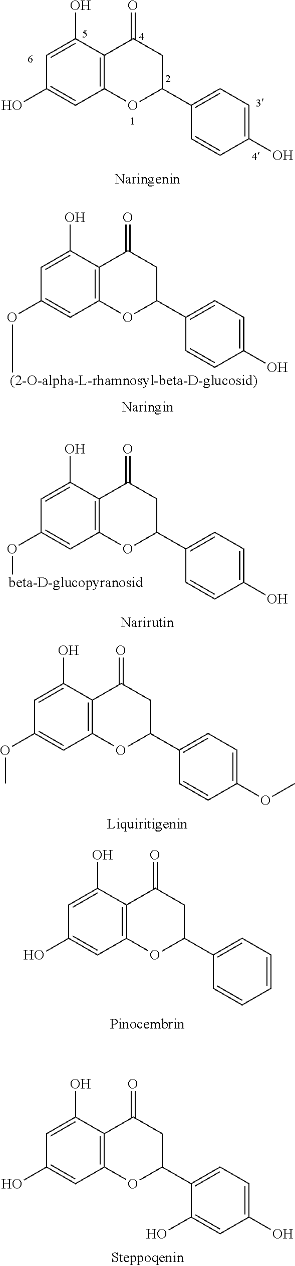

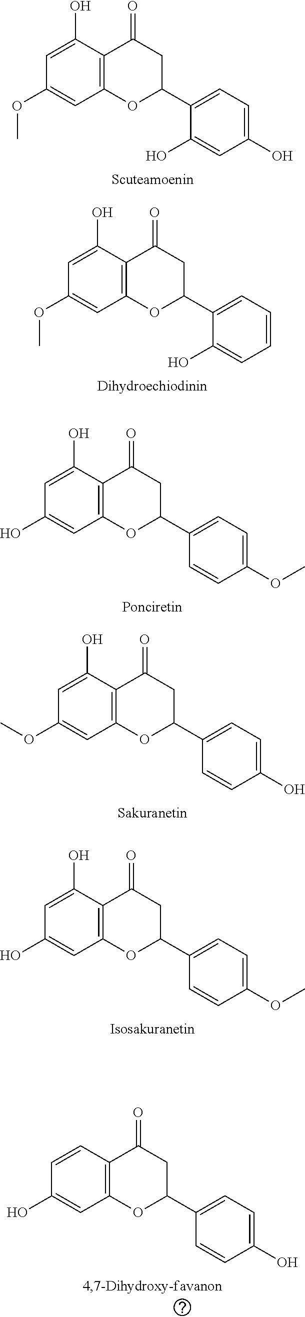

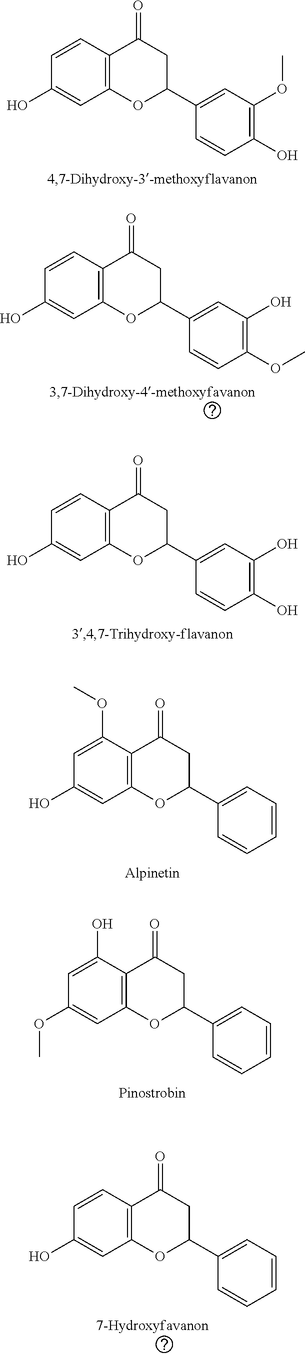 [SMILES: COc1cc(C2CC(=O)c3ccc(O)cc3O2)ccc1O ; COc1ccc(C2CC(=O)c3ccc(O)cc3O2)cc1O ; O=C1CC(c2ccc(O)c(O)c2)Oc2cc(O)ccc21 ; COc1cc(O)cc2c1C(=O)CC(c1ccccc1)O2 ; COc1cc(O)c2c(c1)OC(c1ccccc1)CC2=O ; O=C1CC(c2ccccc2)Oc2cc(O)ccc21]

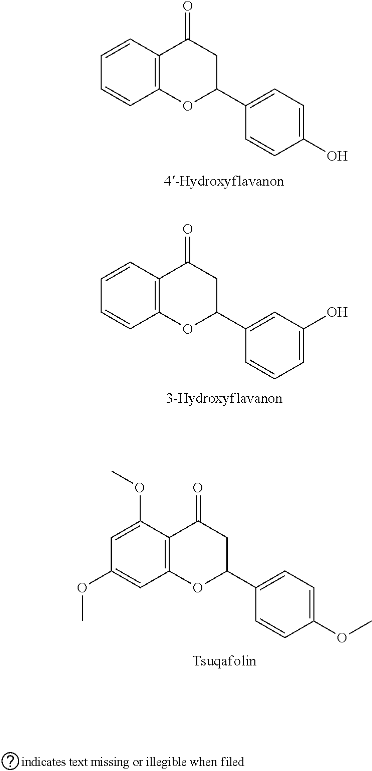 [SMILES: O=C1CC(c2ccc(O)cc2)Oc2ccccc21 ; O=C1CC(c2cccc(O)c2)Oc2ccccc21 ; COc1ccc(C2CC(=O)c3c(OC)cc(OC)cc3O2)cc1]

One embodiment of the present invention relates to a method according to the invention, wherein at least one flavanone and/or at least one chalcone and/or at least one of the corresponding glycosides is provided in step iii), and wherein the at least one flavanone and/or at least one chalcone and/or at least one of the corresponding glycosides is purified or partially purified.

Another embodiment of the present invention relates to a method according to the invention, wherein the at least one dihydrochalcone obtained in step v) is/are selected from the group consisting of butein dihydrochalcone, homobutein dihydrochalcone, 4-O-methylbutein dihydrochalcone, naringenin dihydrochalcone, hesperetin dihydrochalcone, homoeriodictyol dihydrochalcone and eriodictyol dihydrochalcone.

The preferred dihydrochalcones are depicted with their structural formulas as shown below.

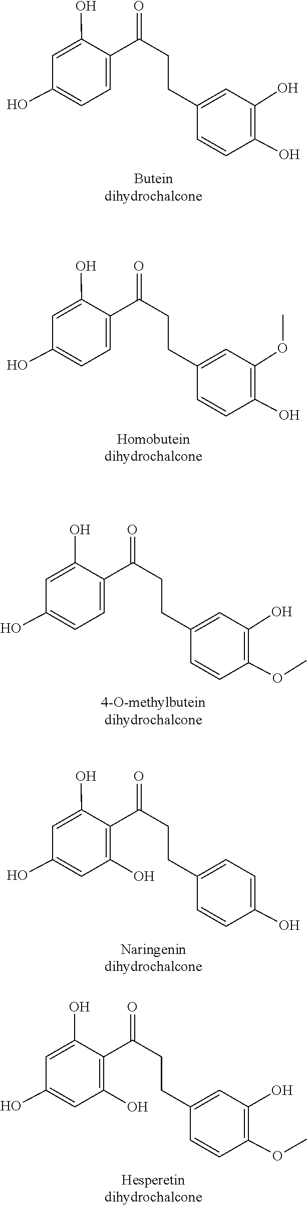 [SMILES: O=C(CCc1ccc(O)c(O)c1)c1ccc(O)cc1O ; COc1cc(CCC(=O)c2ccc(O)cc2O)ccc1O ; COc1ccc(CCC(=O)c2ccc(O)cc2O)cc1O ; O=C(CCc1ccc(O)cc1)c1c(O)cc(O)cc1O ; COc1ccc(CCC(=O)c2c(O)cc(O)cc2O)cc1O]

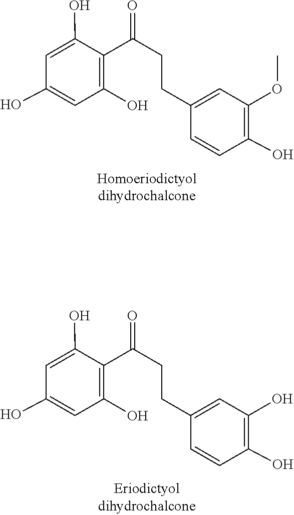 [SMILES: COc1cc(CCC(=O)c2c(O)cc(O)cc2O)ccc1O ; O=C(CCc1ccc(O)c(O)c1)c1c(O)cc(O)cc1O]

All of the embodiments described as preferred above, can be combined with each other interchangeably in terms of the present invention.

Another aspect of the present invention is related to a genetically engineered ene reductase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 169 to 176.

One preferred embodiment of the present invention relates to a genetically engineered ene reductase having a consensus sequence according to SEQ ID NO.: 189.

A consensus sequence describes all genetically engineered enzymes according to the present invention, wherein the variable positions, where an amino acid substitution can be present, are marked by Xaa.

Another preferred embodiment of the present invention relates to a genetically engineered ene reductase, wherein the genetically engineered ene reductase comprises an amino acid at position 276 and/or 290, which is selected from the group consisting of alanine, glycine, serine, asparagine, valine, threonine, leucine, isoleucine, methionine and phenylalanine.

Yet another preferred embodiment of the present invention relates to a genetically engineered ene reductase, wherein the genetically engineered ene reductase comprises an amino acid at position 285, which is selected from the group consisting of leucine, glutamine, threonine, cysteine, phenylalanine aspartate and glutamate.

In another preferred embodiment of the present invention the genetically engineered ene reductase comprises an amino acid substitution at position 285 from valine to glutamine (V285Q) compared to a wild-type sequence.

Ene reductases, which comprise or consist of an amino acid sequence having at least 90%, 91%, 92%, 93%, 94%, 95%, 96%, 97%, 98%, 99% or 100% sequence homology to a sequence selected from the group consisting of SEQ ID Nos 169 to 176 were derived from natural occurring ene reductase AtDBR1 from Arabidopsis thaliana and were genetically engineered to generate new AtDBR1 variants. It was found that especially the variants having at least 90%, 91%, 92%, 93%, 94%, 95%, 96%, 97%, 98%, 99% or 100% sequence homology to a sequence selected from the group consisting of SEQ ID Nos 169 to 176 show an increased activity and selectivity in the conversion of chalcones to dihydrochalcones. These variants show functional amino acid substitutions, which alter the enzyme's specificity and activity.

Also described herein is a genetically engineered chalcone isomerase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 145 to 158.

Preferably, the genetically engineered chalcone isomerase has a consensus sequence according to SEQ ID NO.: 190.

Especially preferably, the genetically engineered chalcone comprises at least one of the following amino acids at the specified positions:
at position 40;
at position 79 alanine, proline, aspartate, glutamate, leucine, valine methionine or isoleucine;
at position 87 lysine, arginine or asparagine;
at position 122 aspartate, asparagine or glutamate;
at position 125 an arginine or glycine

Moreover preferred is a genetically engineered chalcone isomerase, wherein the genetically engineered chalcone isomerase comprises an amino acid at position 79 or 125, which is selected from the group consisting of alanine, glycine, proline, isoleucine, valine, methionine, aspartate, glutamate, and argininelt was surprisingly found that the genetically engineered chalcone isomerase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 145 to 158 show a superior performance in catalysing the reaction from flavanone to chalcone. It was especially surprising that the chalcone isomerases according to the invention have their reaction equivalent on the site of the chalcone; they mainly catalyse the reaction from flavanone to chalcone. The so obtained chalcone can then be catalysed to a dihydrochalcone by an ene reductase according to the present invention and as provided in step i) of the method according to the present invention.

Preferably, the genetically engineered chalcone isomerase comprises or consists of an amino acid sequence having at least 90%, 91%, 92%, 93%, 94%, 95%, 96%, 97%, 98%, 99% or 100% sequence homology to a sequence selected from the group consisting of SEQ ID NOs 154, 157, 145, 152, 155, 153, 147, more preferably selected from the group consisting of SEQ ID NOs 157, 154 and 145.

In a further preferred embodiment, the sequence of the genetically engineered chalcone isomerase is derived from Eubacterium ramulus having at least 90%, 91%, 92%, 93%, 94%, 95%, 96%, 97%, 98%, 99% or 100% sequence homology to a sequence selected from the group consisting of SEQ ID NOs 85 to 98.

All of the above described embodiments of enzymes can be used in terms of the method according to the invention as described above.

Yet another aspect of the present invention relates to a transgenic microorganism comprising a nucleic acid sequence encoding a genetically engineered ene reductase according to the present invention.

One preferred embodiment of the invention relates to a transgenic microorganism according the invention, wherein the microorganism is selected from the group consisting of Escherichia coli spp., such as E. coli BL21, E. coli MG1655, preferably E. coli W3110, Bacillus spp., such as Bacillus licheniformis, Bacillus subitilis, or Bacillus amyloliquefaciens, Saccharomyces spp., preferably S. cerevesiae, Hansenula or Komagataella spp., such as. K. phaffii and H. polymorpha, preferably K. phaffii, Yarrowia spp. such as Y. lipolytica, Kluyveromyces spp, such as K. lactis.

One aspect of the present invention relates to a vector, preferably a plasmid vector, comprising
at least one nucleic acid sequence encoding an ene reductase having at least 90%, 91%, 92%, 93%, 94%, 95%, 96%, 97%, 98%, 99% or 100% sequence homology to an amino acid sequence selected from the group consisting of SEQ ID NOs 24 to 46 and 169 to 176,
and optionally
at least one nucleic acid sequence encoding a genetically engineered chalcone isomerase having at least 90%, 91%, 92%, 93%, 94%, 95%, 96%, 97%, 98%, 99% or 100% sequence homology to a sequence selected from the group consisting of SEQ ID NOs 145 to 158.

In one preferred embodiment of the vector according to the invention, the vector comprises
at least one nucleic acid sequence encoding an ene reductase having at least 90%, 91%, 92%, 93%, 94%, 95%, 96%, 97%, 98%, 99% or 100% sequence homology to an amino acid sequence selected from the group consisting of SEQ ID NOs 24 to 46 and 169 to 176, and
at least one nucleic acid sequence encoding a genetically engineered chalcone isomerase having at least 90%, 91%, 92%, 93%, 94%, 95%, 96%, 97%, 98%, 99% or 100% sequence homology to a sequence selected from the group consisting of SEQ ID NOs 145 to 158.

One aspect of the present invention relates to a vector system comprising two vectors, wherein the first vector comprises
at least one nucleic acid sequence encoding an ene reductase having at least 90%, 91%, 92%, 93%, 94%, 95%, 96%, 97%, 98%, 99% or 100% sequence homology to an amino acid sequence selected from the group consisting of SEQ ID NOs 24 to 46 and 169 to 176,
and the second vector
at least one nucleic acid sequence encoding a genetically engineered chalcone isomerase having at least 90%, 91%, 92%, 93%, 94%, 95%, 96%, 97%, 98%, 99% or 100% sequence homology to a sequence selected from the group consisting of SEQ ID NOs 145 to 158.

Further embodiments of the transgenic microorganism or vector as described above, become apparent when studying the above described preferred embodiments of a method according to the present invention and, thus, apply accordingly in connection with a transgenic microorganism or vector according to the present invention.

Another aspect of the present invention relates to the use of at least one ene reductase according to the present invention and/or at least one transgenic microorganism according to the present invention and/or at least one vector according to the present invention, preferably as described above as preferred, in the biocatalytical manufacturing of dihydrochalcones, preferably in a method according to the present invention.

The invention is further characterized by illustrative, non-limiting examples.

### BRIEF DESCRIPTION OF FIGURES

FIG. 1 shows a LC chromatogram of the biotransformation of butein using lysate supernatant of E. coli BL21(DE3) cells expressing CHI and AtDBR1 (dotted line) or expressing CHI only (solid line).

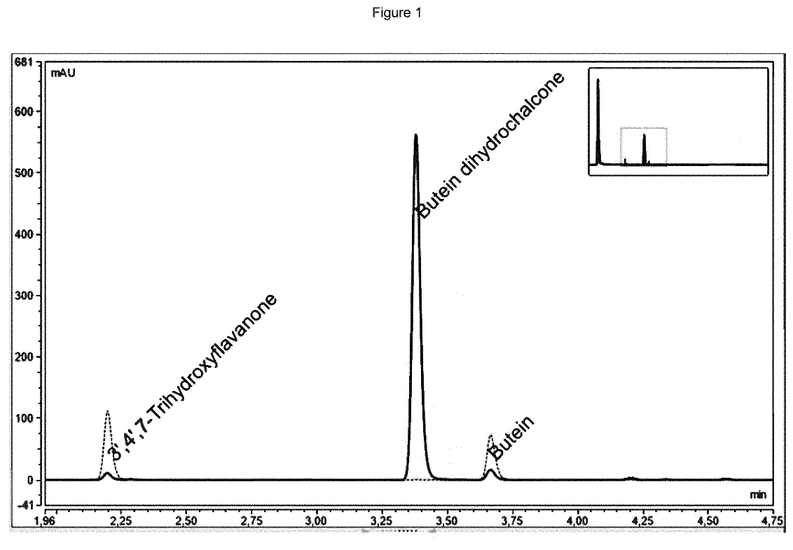

FIG. 2 shows a: LC chromatogram of the biotransformation of homobutein using lysate supernatant of E. coli BL21 (DE3) cells expressing CHI and AtDBR1 (dotted line) or expressing CHI only (solid line).

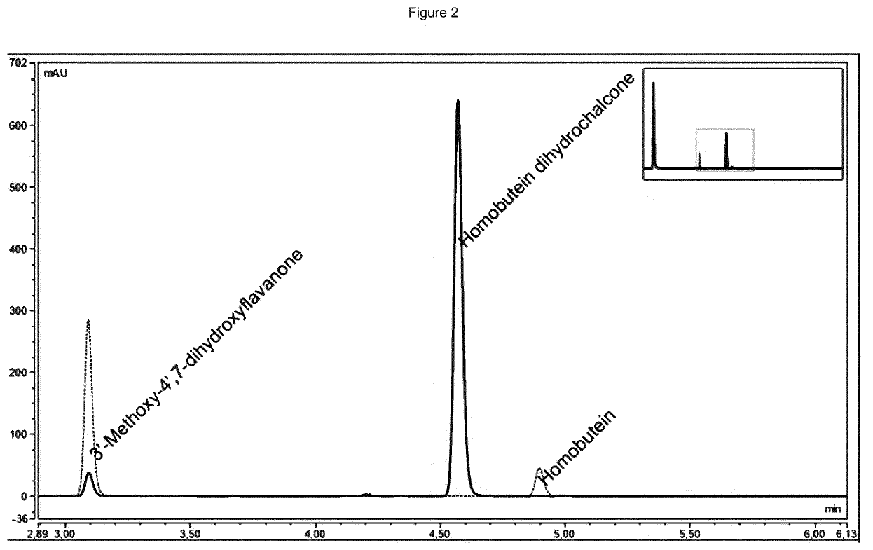

FIG. 3 shows a LC chromatogram of biotransformation of 4-O-methyl butein using lysate supernatant of E. coli BL21(DE3) cells expressing CHI and AtDBR1 (dotted line) or expressing CHI only (solid line).

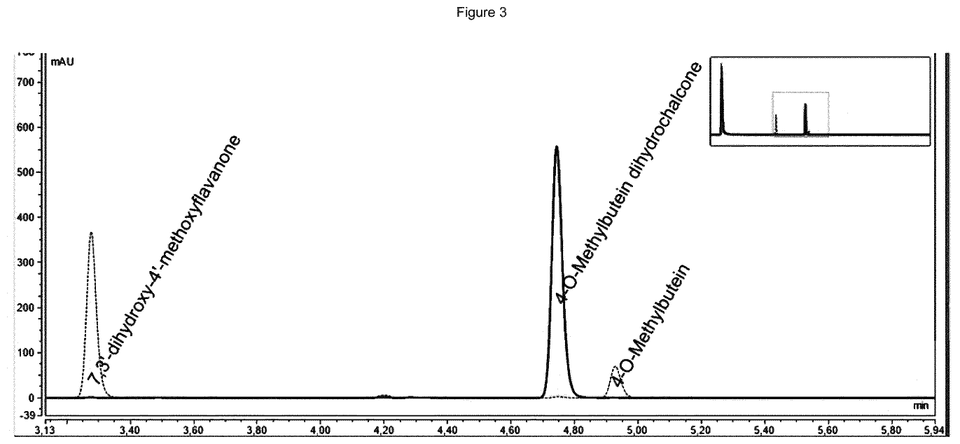

FIG. 4 shows a LC-MS chromatogram of the biotransformation of naringenin chalcone using lysate supernatant of E. coli BL21 (DE3) cells expressing AtDBR1.

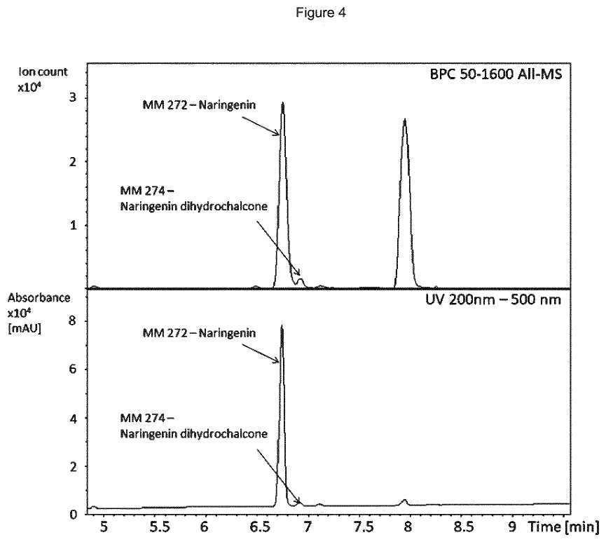

FIG. 5 shows a LC-MS chromatogram of the biotransformation of hesperetin chalcone using lysate supernatant of E. coli BL21 (DE3) cells expressing AtDBR1.

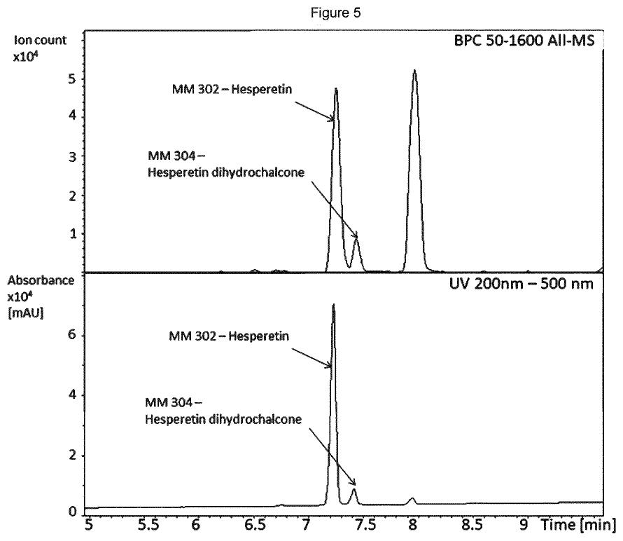

FIG. 6 shows a LC-MS chromatogram of biotransformation of eriodictyol chalcone using lysate supernatant of E. coli BL21 (DE3) cells expressing AtDBR1.

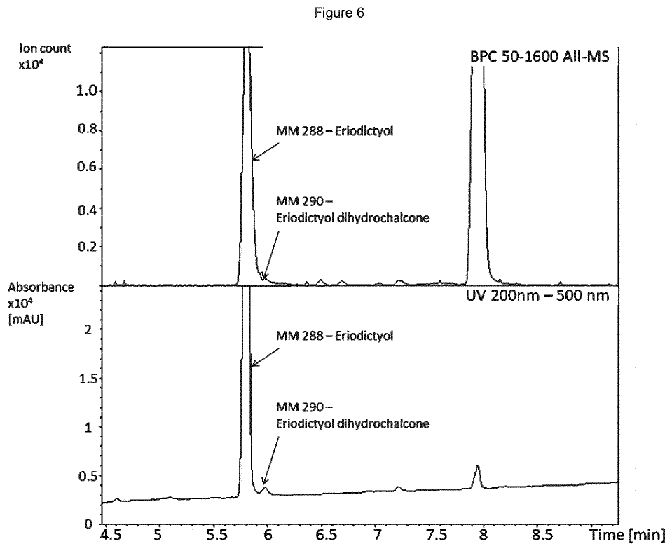

FIG. 7 shows the product butein dihydrochalcone formation of lysate supernatants of E. coli BL21 (DE3) cells expressing different ene reductases incubated with butein.

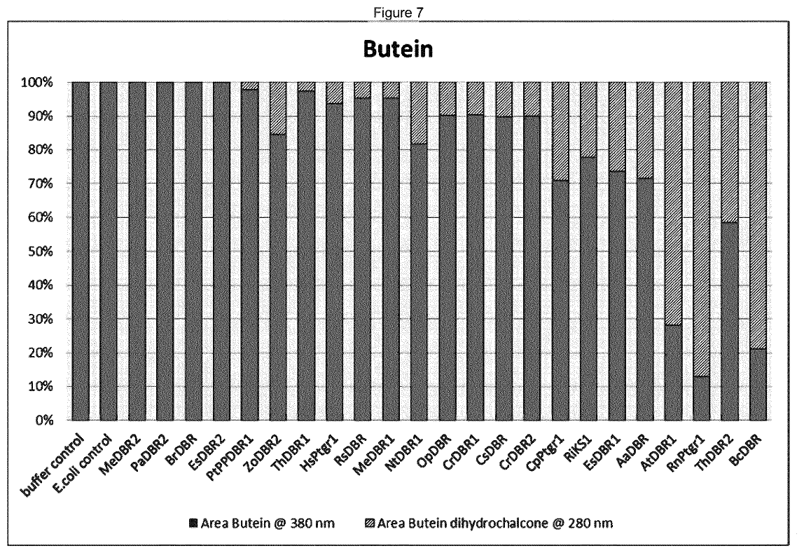

FIG. 8 shows the product homobutein dihydrochalcone formation of lysate supernatants of E. coli BL21 (DE3) cells expressing different ene reductases incubated with homobutein.

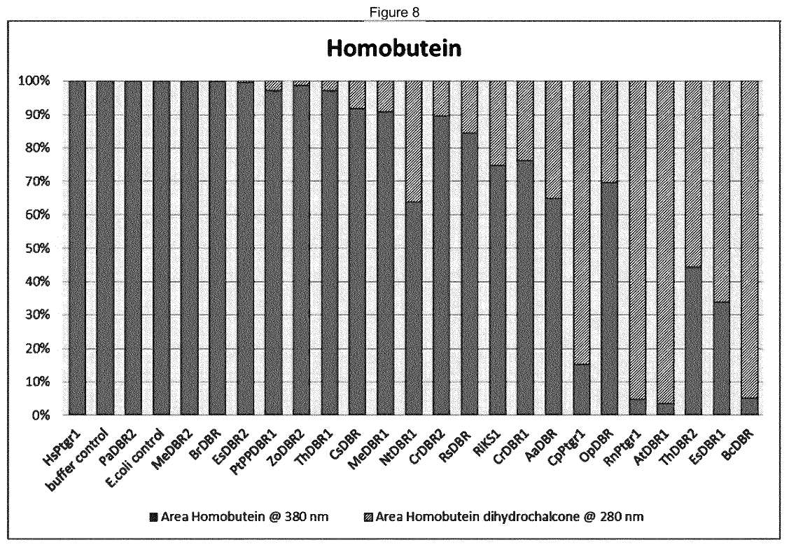

FIG. 9 shows the product 4-O-methyl butein dihydrochalcone formation of lysate supernatants of E. coli BL21 (DE3) cells expressing different ene reductases incubated with 4-O-methyl butein.

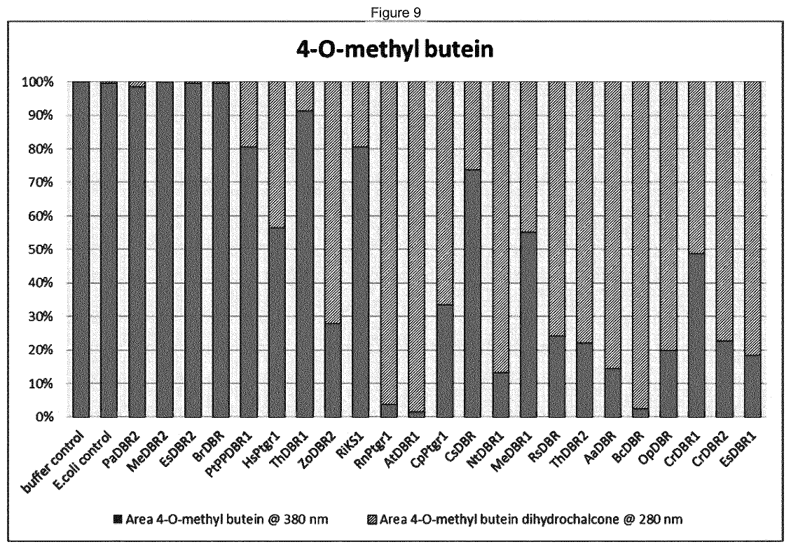

FIG. 10 shows the product naringenin dihydrochalcone formation of lysate supernatants of E. coli BL21 (DE3) cells expressing different ene reductases incubated with Naringenin chalcone.

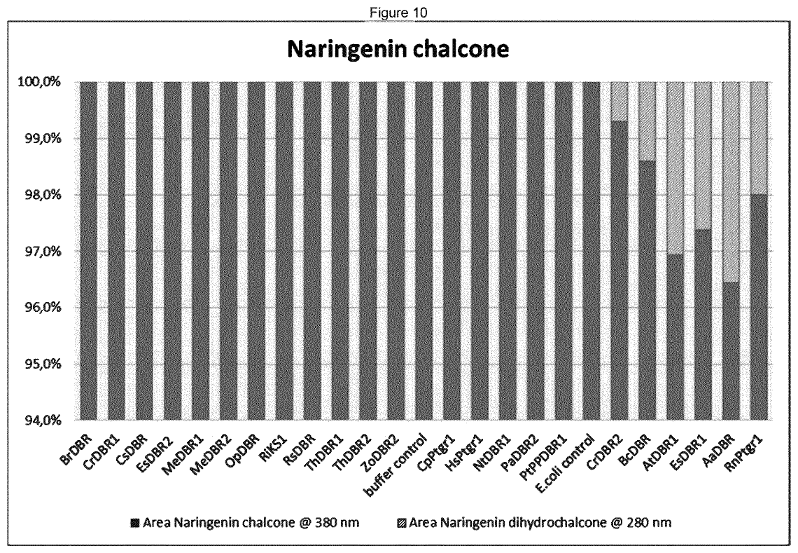

FIG. 11 shows the product hesperetin dihydrochalcone formation of lysate supernatants of E. coli BL21 (DE3) cells expressing different ene reductases incubated with hesperetin chalcone.

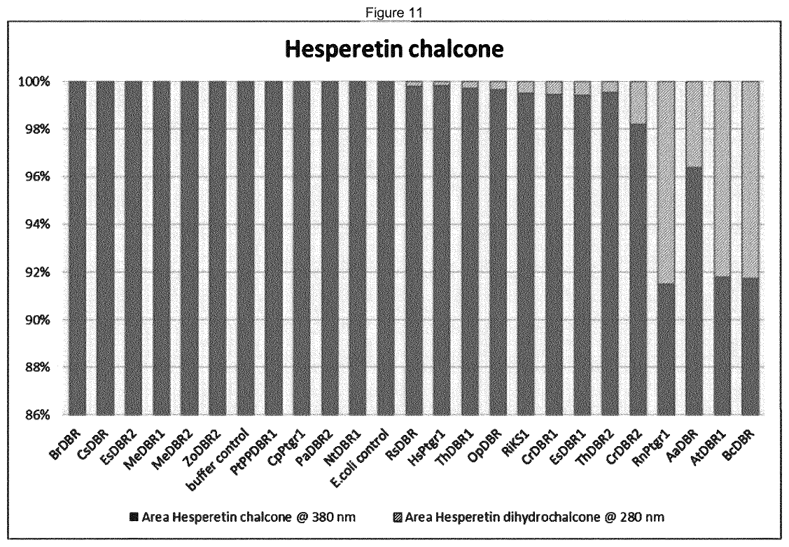

FIG. 12 shows the product eriodictyol dihydrochalcone formation of lysate supernatants of E. coli BL21 (DE3) cells expressing different ene reductases incubated with eriodictyol chalcone.

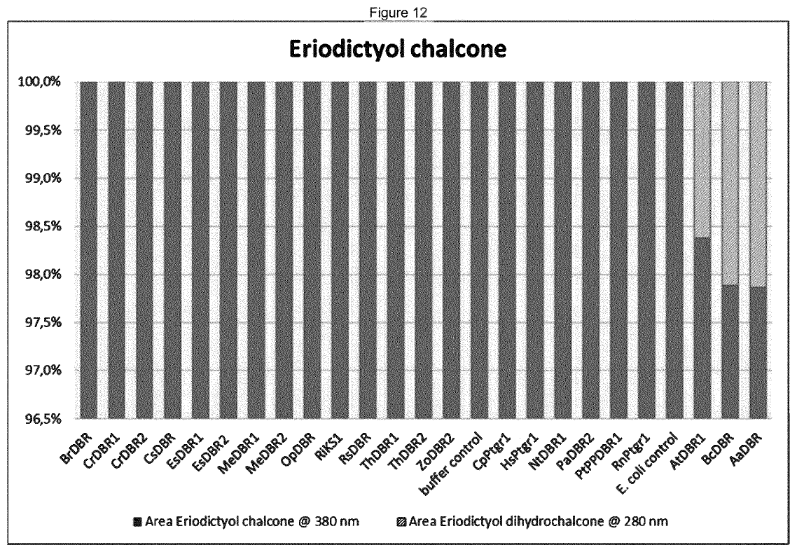

FIG. 13 shows the product homoeriodictyol dihydrochalcone formation of lysate supernatants of E. coli BL21 (DE3) cells expressing different ene reductases incubated with homoeriodictyol chalcone.

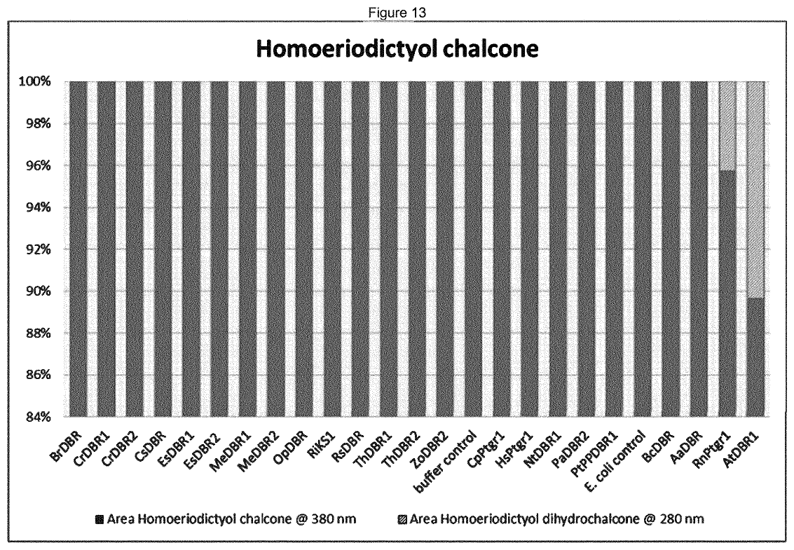

FIG. 14 shows the specific activities of CHIs and CHIera variants towards different chalcones.

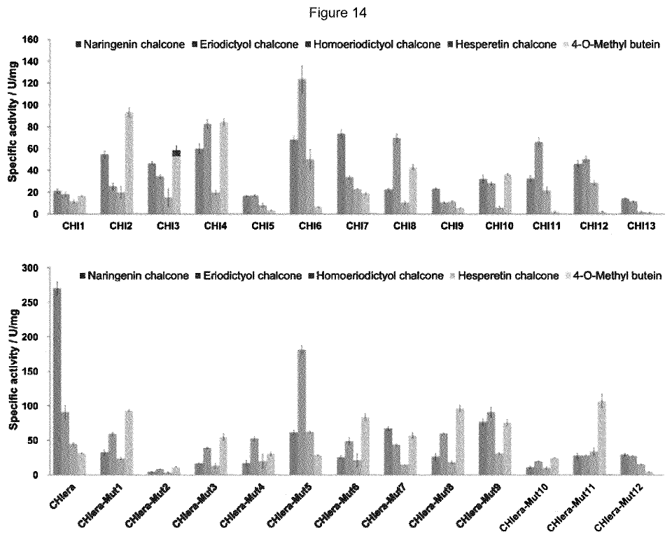

FIG. 15 shows the dihydrochalcone product formation of purified AtDBR1 incubated with different chalcones.

FIG. 16 shows the product hesperetin dihydrochalcone formation of lysate supernatants of E. coli BL21 (DE3) cells expressing different AtDBR1 variants incubated with hesperetin chalcone.

FIG. 17 shows LC-MS chromatograms of hesperetin incubations A) without enzyme in 50 mM phosphate buffer pH 6.0 for 90 min, B) with purified AtDBR1 for 0 min, C) with purified AtDBR1 for 90 min.

### SHORT DESCRIPTION OF SEQUENCES

| SEQ ID NO. | Description |
|---|---|
| 1 | Coding sequence of ene reductase from Arabidopsis alpina |
| 2 | Coding sequence of ene reductase from Arabidopsis thaliana |
| 3 | Coding sequence of ene reductase from Brassica cretica |
| 4 | Coding sequence of ene reductase from Brassica rapa |
| 5 | Coding sequence of ene reductase from Cavia porcellus |
| 6 | Coding sequence 1 of ene reductase from Capsella rubella |
| 7 | Coding sequence 2 of ene reductase from Capsella rubella |
| 8 | Coding sequence of ene reductase from Camelina sativa |
| 9 | Coding sequence 1 of ene reductase from Eutrema salsugineum |
| 10 | Coding sequence 2 of ene reductase from Eutrema salsugineum |
| 11 | Coding sequence of ene reductase from Homo sapiens |
| 12 | Coding sequence 1 of ene reductase from Microthlaspi erraticum |
| 13 | Coding sequence 2 of ene reductase from Microthlaspi erraticum |
| 14 | Coding sequence of ene reductase from Nicotiana tabacum |
| 15 | Coding sequence of ene reductase from Olimarabidopsis pumila |
| 16 | Coding sequence of ene reductase from Plagiochasma appendiculatum |
| 17 | Coding sequence of ene reductase from Pinus taeda |
| 18 | Coding sequence of ene reductase from Rubus idaeus |
| 19 | Coding sequence of ene reductase from Rattus norvegicus |
| 20 | Coding sequence of ene reductase from Raphanus sativus |
| 21 | Coding sequence 1 of ene reductase from Tarenaya hassleriana |
| 22 | Coding sequence 2 of ene reductase from Tarenaya hassleriana |
| 23 | Coding sequence of ene reductase from Zingiber officinale |
| 24 | Ene reductase from Arabidopsis alpina |
| 25 | Ene reductase from Arabidopsis thaliana |
| 26 | Ene reductase from Brassica cretica |
| 27 | Ene reductase from Brassica rapa |
| 28 | Ene reductase from Cavia porcellus |
| 29 | Ene reductase 1 from Capsella rubella |
| 30 | Ene reductase 2 from Capsella rubella |
| 31 | Ene reductase from Camelina sativa |
| 32 | Ene reductase 1 from Eutrema salsugineum |
| 33 | Ene reductase 2 from Eutrema salsugineum |
| 34 | Ene reductase from Homo sapiens |
| 35 | Ene reductase 1 from Microthlaspi erraticum |
| 36 | Ene reductase 2 from Microthlaspi erraticum |
| 37 | Ene reductase from Nicotiana tabacum |
| 38 | Ene reductase from Olimarabidopsis pumila |
| 39 | Ene reductase from Plagiochasma appendiculatum |
| 40 | Ene reductase from Pinus taeda |
| 41 | Ene reductase from Rubus idaeus |
| 42 | Ene reductase from Rattus norvegicus |
| 43 | Ene reductase from Raphanus sativus |
| 44 | Ene reductase 1 from Tarenaya hassleriana |
| 45 | Ene reductase 2 from Tarenaya hassleriana |
| 46 | Ene reductase from Zingiber officinale |
| 47 | Coding sequence for chalcone isomerase from Acetoanaerobium noterae |
| 48 | Chalcone isomerase from Acetoanaerobium noterae |
| 49 | Sequence of Forward primer |
| 50 | Sequence of Reverse primer |
| 51 | Sequence of Forward primer |
| 52 | Sequence of Reverse primer |
| 53 | Coding sequence of Chalcone isomerase from Fusibacter sp. 3D3 |
| 54 | Coding sequence of Chalcone isomerase from Tepidanaerobacter |
| 55 | Coding sequence of Chalcone isomerase from Clostridium sp. JN500901 |
| 56 | Coding sequence of Chalcone isomerase from Acetoanaerobium noterae |
| 57 | Coding sequence of Chalcone isomerase from Parasporobacterium |
| 58 | Coding sequence of Chalcone isomerase from Lachnoclostridium sp. |
| 59 | Coding sequence of Chalcone isomerase from Clostridium sp. SY8519 |
| 60 | Coding sequence of Chalcone isomerase from Roseburia sp. |
| 61 | Coding sequence of Chalcone isomerase from Roseburia sp. OM02-15 |
| 62 | Coding sequence of Chalcone isomerase from Clostridium sp. SY8519 |
| 63 | Coding sequence of Chalcone isomerase from Eubacterium sp. |
| 64 | Coding sequence of Chalcone isomerase from Holophaga foetida |
| 65 | Coding sequence of Chalcone isomerase from Lactobacillus sp. 54-2 |
| 66 | Coding sequence of Chalcone isomerase from Butyrivibrio sp. AC2005 |
| 67 | Coding sequence of Chalcone isomerase from Clostridioides difficile |
| 68 | Coding sequence of Chalcone isomerase from Eubacterium ramulus |
| 69 | Chalcone isomerase from Fusibacter sp. 3D3 |
| 70 | Chalcone isomerase from Tepidanaerobacter acetatoxydans |
| 71 | Chalcone isomerase from Clostridium sp. JN500901 |
| 72 | Chalcone isomerase from Acetoanaerobium noterae |
| 73 | Chalcone isomerase from Parasporobacterium paucivorans |
| 74 | Chalcone isomerase from Lachnoclostridium sp. |
| 75 | Chalcone isomerase from Clostridium sp. SY8519 |
| 76 | Chalcone isomerase from Roseburia sp. |
| 77 | Chalcone isomerase from Roseburia sp. OM02-15 |
| 78 | Chalcone isomerase from Clostridium sp. SY8519 |
| 79 | Chalcone isomerase from Eubacterium sp. |
| 80 | Chalcone isomerase from Holophaga foetida |
| 81 | Chalcone isomerase from Lactobacillus sp. 54-2 |
| 82 | Chalcone isomerase from Butyrivibrio sp. AC2005 |
| 83 | Chalcone isomerase from Clostridioides difficile |
| 84 | Chalcone isomerase from Eubacterium ramulus |
| 85 | Coding sequence of Chalcone isomerase variant 1 from Eubacterium ramulus |
| 86 | Coding sequence of Chalcone isomerase variant 2 from Eubacterium ramulus |
| 87 | Coding sequence of Chalcone isomerase variant 3 from Eubacterium ramulus |
| 88 | Coding sequence of Chalcone isomerase variant 4 from Eubacterium ramulus |
| 89 | Coding sequence of Chalcone isomerase variant 5 from Eubacterium ramulus |
| 90 | Coding sequence of Chalcone isomerase variant 6 from Eubacterium ramulus |
| 91 | Coding sequence of Chalcone isomerase variant 7 from Eubacterium ramulus |
| 92 | Coding sequence of Chalcone isomerase variant 8 from Eubacterium ramulus |
| 93 | Coding sequence of Chalcone isomerase variant 9 from Eubacterium ramulus |
| 94 | Coding sequence of Chalcone isomerase variant 10 from Eubacterium |
| 95 | Coding sequence of Chalcone isomerase variant 11 from Eubacterium |
| 96 | Coding sequence of Chalcone isomerase variant 12 from Eubacterium |
| 97 | Coding sequence of Chalcone isomerase variant 13 from Eubacterium |
| 98 | Coding sequence of Chalcone isomerase variant 14 from Eubacterium |
| 99 | Nucleotide sequence of forward primer of pair 1 |
| 100 | Nucleotide sequence of reverse primer of pair 1 |
| 101 | Nucleotide sequence of forward primer of pair 2 |
| 102 | Nucleotide sequence of reverse primer of pair 2 |
| 103 | Nucleotide sequence of forward primer of pair 3 |
| 104 | Nucleotide sequence of reverse primer of pair 3 |
| 105 | Nucleotide sequence of forward primer of pair 4 |
| 106 | Nucleotide sequence of reverse primer of pair 4 |
| 107 | Nucleotide sequence of forward primer of pair 5 |
| 108 | Nucleotide sequence of reverse primer of pair 5 |
| 109 | Nucleotide sequence of forward primer of pair 6 |
| 110 | Nucleotide sequence of reverse primer of pair 6 |
| 111 | Nucleotide sequence of forward primer of pair 7 |
| 112 | Nucleotide sequence of reverse primer of pair 7 |
| 113 | Nucleotide sequence of forward primer of pair 8 |
| 114 | Nucleotide sequence of reverse primer of pair 8 |
| 115 | Nucleotide sequence of forward primer of pair 9 |
| 116 | Nucleotide sequence of reverse primer of pair 9 |
| 117 | Nucleotide sequence of forward primer of pair 10 |
| 118 | Nucleotide sequence of reverse primer of pair 10 |
| 119 | Nucleotide sequence of forward primer of pair 11 |
| 120 | Nucleotide sequence of reverse primer of pair 11 |
| 121 | Nucleotide sequence of forward primer of pair 12 |
| 122 | Nucleotide sequence of reverse primer of pair 12 |
| 123 | Nucleotide sequence of forward primer of pair 13 |
| 124 | Nucleotide sequence of reverse primer of pair 13 |
| 125 | Nucleotide sequence of forward primer of pair 14 |
| 126 | Nucleotide sequence of reverse primer of pair 14 |
| 127 | Nucleotide sequence of forward primer of pair 15 |
| 128 | Nucleotide sequence of reverse primer of pair 15 |
| 129 | Nucleotide sequence of forward primer of pair 16 |
| 130 | Nucleotide sequence of reverse primer of pair 16 |
| 131 | Nucleotide sequence of forward primer of pair 17 |
| 132 | Nucleotide sequence of reverse primer of pair 17 |
| 133 | Nucleotide sequence of forward primer of pair 18 |
| 134 | Nucleotide sequence of reverse primer of pair 18 |
| 135 | Nucleotide sequence of forward primer of pair 19 |
| 136 | Nucleotide sequence of reverse primer of pair 19 |
| 137 | Nucleotide sequence of forward primer of pair 20 |
| 138 | Nucleotide sequence of reverse primer of pair 20 |
| 139 | Nucleotide sequence of forward primer of pair 21 |
| 140 | Nucleotide sequence of reverse primer of pair 21 |
| 141 | Nucleotide sequence of forward primer of pair 22 |
| 142 | Nucleotide sequence of reverse primer of pair 22 |
| 143 | Nucleotide sequence of forward primer of pair 23 |
| 144 | Nucleotide sequence of reverse primer of pair 23 |
| 145 | Amino acid sequence of Chalcone isomerase variant 1 from Eubacterium |
| 146 | Amino acid sequence of Chalcone isomerase variant 2 from Eubacterium |
| 147 | Amino acid sequence of Chalcone isomerase variant 3 from Eubacterium |
| 148 | Amino acid sequence of Chalcone isomerase variant 4 from Eubacterium |
| 149 | Amino acid sequence of Chalcone isomerase variant 5 from Eubacterium |
| 150 | Amino acid sequence of Chalcone isomerase variant 6 from Eubacterium |
| 151 | Amino acid sequence of Chalcone isomerase variant 7 from Eubacterium |
| 152 | Amino acid sequence of Chalcone isomerase variant 8 from Eubacterium |
| 153 | Amino acid sequence of Chalcone isomerase variant 9 from Eubacterium |
| 154 | Amino acid sequence of Chalcone isomerase variant 10 from Eubacterium |
| 155 | Amino acid sequence of Chalcone isomerase variant 11 from Eubacterium |
| 156 | Amino acid sequence of Chalcone isomerase variant 12 from Eubacterium |
| 157 | Amino acid sequence of Chalcone isomerase variant 13 from Eubacterium |
| 158 | Amino acid sequence of Chalcone isomerase variant 14 from Eubacterium |
| 159 | Coding sequence of ene reductase from Arabidopsis thaliana |
| 160 | Amino acid sequence of ene reductase from Arabidopsis thaliana |
| 161 | Coding sequence of AtDBR1 variant V285Q |
| 162 | Coding sequence of AtDBR1 variant V285T |
| 163 | Coding sequence of AtDBR1 variant V285D |
| 164 | Coding sequence of AtDBR1 variant V285L |
| 165 | Coding sequence of AtDBR1 variant Y81F |
| 166 | Coding sequence of AtDBR1 variant Y276A |
| 167 | Coding sequence of AtDBR1 variant Y290A |
| 168 | Coding sequence of AtDBR1 variant Y290F |
| 169 | Amino acid sequence of AtDBR1 variant V285Q |
| 170 | Amino acid sequence of AtDBR1 variant V285T |
| 171 | Amino acid sequence of AtDBR1 variant V285D |
| 172 | Amino acid sequence of AtDBR1 variant V285L |
| 173 | Amino acid sequence of AtDBR1 variant Y81F |
| 174 | Amino acid sequence of AtDBR1 variant Y276A |
| 175 | Amino acid sequence of AtDBR1 variant Y290A |
| 176 | Amino acid sequence of AtDBR1 variant Y290F |
| 177 | Nucleotide sequence of reverse primer of pairs 1, 2, 3 and 4 |
| 178 | Nucleotide sequence of forward primer of pair 1 |
| 179 | Nucleotide sequence of forward primer of pair 2 |
| 180 | Nucleotide sequence of forward primer of pair 3 |
| 181 | Nucleotide sequence of forward primer of pair 4 |
| 182 | Nucleotide sequence of reverse primer of pair 5 |
| 183 | Nucleotide sequence of forward primer of pair 5 |
| 184 | Nucleotide sequence of reverse primer of pair 6 |
| 185 | Nucleotide sequence of forward primer of pair 6 |
| 186 | Nucleotide sequence of reverse primer of pairs 7 and 8 |
| 187 | Nucleotide sequence of forward primer of pair 7 |
| 188 | Nucleotide sequence of forward primer of pair 8 |
| 189 | Consensus sequence of ene reductase |
| 190 | Consensus sequence of chalcone isomerase |

### EXAMPLES

### 1. Transformation of Plasmid DNA Into Escherichia coli Cells

Plasmid DNA was transformed into chemically competent Escherichia coli (E. coli) DH5α cells (New England Biolabs, Frankfurt am Main, Germany) for plasmid propagation. For generation of expression strains, plasmid DNA was transformed into chemically competent E. coli BL21 (DE3) cells.

50 μL of the respective E. coli strain were incubated on ice for 5 minutes. After addition of 1 μl of plasmid DNA, the suspension was mixed and incubated for 30 minutes on ice. Transformation was performed by incubating the cell suspension for 45 s at 42° C. in a thermoblock followed by incubation on ice for 2 minutes. After addition of 350 μl SOC Outgrowth Medium (New England Biolabs, Frankfurt am Main, Germany) cells were incubated at 37° C. and 200 rpm for 1 hour. Subsequently, the cell suspension was spread out on LB-Agar plates (Car Roth GmbH, Karlsruhe, Germany) containing the respective antibiotic and incubated for 16 hours at 37° C.

### 2. Generation of Expression Plasmids

The SEQ ID NO 47, which encodes SEQ ID NO 48, was cloned into vector pCDFDuet-1 to obtain vector pCDFDuet-1CHI. Vector pCDFDuet-1 with SEQ ID NO 49 and SEQ ID NO 50 as well as SEQ ID NO 56 with SEQ ID NO 51 and SEQ ID NO 52 were amplified by polymerase chain reaction (PCR) according to common practice known to experts, the reaction solutions were mixed in a ratio of 1:1 and 1.5 μl of the mixture was transformed into E. coli DH5α after 1 h incubation at 37° C. as described in Example 1. Vector pCDFDuet-1CHI is transformed as described in Example 1 into E. coli BL21 (DE3).

The sequences SEQ ID NO 1 to SEQ ID NO 23 coding for SEQ ID NO 24 to SEQ ID NO 46, respectively, were synthesized and cloned into the pET28a vector between NcoI and XhoI restriction sites (Twist BioScience, San Francisco, USA) to obtain plasmids listed in Table 1. These expression vectors are transformed as described in Example 1 into E. coli BL21 (DE3) cells or E. coli BL21 (DE3) cells containing plasmid pCDFDuet-1CHI.

TABLE 1
Obtained plasmids and the enzyme are obtained of.

| Plasmid | Insert SEQ ID NO. | Organism |
|---|---|---|
| pET28a_AaDBR | 1 | Arabidopsis alpina |
| pET28a_AtDBR1 | 2 | Arabidopsis thaliana |
| pET28a_BcDBR | 3 | Brassica cretica |
| pET28a_BrDBR | 4 | Brassica rapa |
| pET28a_CpPtgr1 | 5 | Cavia porcellus |
| pET28a_CrDBR1 | 6 | Capsella rubella |
| pET28a_CrDBR2 | 7 | Capsella rubella |
| pET28a_CsDBR | 8 | Camelina sativa |
| pET28a_EsDBR1 | 9 | Eutrema salsugineum |
| pET28a_EsDBR2 | 10 | Eutrema salsugineum |
| pET28a_HsPtgr1 | 11 | Homo sapiens |
| pET28a_MeDBR1 | 12 | Microthlaspi erraticum |
| pET28a_MeDBR2 | 13 | Microthlaspi erraticum |
| pET28a_NtDBR1 | 14 | Nicotiana tabacum |
| pET28a_OpDBR | 15 | Olimarabidopsis pumila |
| pET28a_PaDBR2 | 16 | Plagiochasma |
| | | appendiculatum |
| pET28a_PtPPDBR1 | 17 | Pinus taeda |
| pET28a_RiKS1 | 18 | Rubus idaeus |
| pET28a_RnPtgr1 | 19 | Rattus norvegicus |
| pET28a_RsDBR | 20 | Raphanus sativus |
| pET28a_ThDBR1 | 21 | Tarenaya hassleriana |
| pET28a_ThDBR2 | 22 | Tarenaya hassleriana |
| pET28a_ZoDBR2 | 23 | Zingiber officinale |

The sequences SEQ ID NO 53 to SEQ ID NO 68 coding for SEQ ID NO 69 to SEQ ID NO 84, respectively, were synthesized and cloned into the pET28b vector between NdeI and BamHI restriction sites (BioCat, Heidelberg, Germany) to obtain plasmids listed in Table 2. These expression vectors are transformed as described in Example 1 into E. coli BL21 (DE3) cells.

TABLE 2
Obtained plasmids and organisms, the
enzyme sequences are obtained of.

| Plasmid | Insert SEQ ID NO | Organism |
|---|---|---|
| pET28b_CHI1 | 53 | Fusibacter sp. 3D3 |
| pET28b_CHI2 | 54 | Tepidanaerobacter |
| | | acetatoxydans |
| pET28b_CHI3 | 55 | Clostridium sp. JN500901 |
| pET28b_CHI4 | 56 | Acetoanaerobium noterae |
| pET28b_CHI5 | 57 | Parasporobacterium |
| | | paucivorans |
| pET28b_CHI6 | 58 | Lachnoclostridium sp. |
| pET28b_CHI7 | 59 | Clostridium sp. SY8519 |
| pET28b_CHI8 | 60 | Roseburia sp. |
| pET28b_CHI9 | 61 | Roseburia sp. OM02-15 |
| pET28b_CHI10 | 62 | Clostridium sp. SY8519 |
| pET28b_CHI11 | 63 | Eubacterium sp. |
| pET28b_CHI12 | 64 | Holophaga foetida |
| pET28b_CHI13 | 65 | Lactobacillus sp. 54-2 |
| pET28b_CHI14 | 66 | Butyrivibrio sp. AC2005 |
| pET28b_CHI15 | 67 | Clostridioides difficile |
| pET28b_CHIera | 68 | Eubacterium ramulus |

By artificial design, certain mutants of CHIera were created to optimize the activity and/or specificity of the CHIera enzyme. These CHIera mutant variants correspond to SEQ ID NOs: 85 to SEQ ID NO: 96, which codes for SEQ ID Nos: 145 to SEQ ID NO: 158 and were generated via site-directed mutagenesis using the QuikChange kit (Agilent, USA). For this, vector pET28b_CHIera containing SEQ ID NO 68 was amplified by polymerase chain reaction (PCR) according to manufacturer's manual with one primer pair of same pair number from sequences SEQ ID NO 99 to SEQ ID NO 143, respectively. After digestion with 1 μL DpnI for 1 h at 37° C., the mixture was transformed into E. coli DH5α as described in Example 1 to obtain plasmids listed in Table 3.

TABLE 3
Obtained plasmids and organisms, the
enzyme sequences are obtained of.

| Plasmid | Insert SEQ ID NO | Organism |
|---|---|---|
| pET28b_CHIₑᵣₐMut1 | 85 | Eubacterium ramulus |
| pET28b_CHIₑᵣₐMut2 | 86 | Eubacterium ramulus |
| pET28b_CHIₑᵣₐMut3 | 87 | Eubacterium ramulus |
| pET28b_CHIₑᵣₐMut4 | 88 | Eubacterium ramulus |
| pET28b_CHIₑᵣₐMut5 | 89 | Eubacterium ramulus |
| pET28b_CHIₑᵣₐMut6 | 90 | Eubacterium ramulus |
| pET28b_CHIₑᵣₐMut7 | 91 | Eubacterium ramulus |
| pET28b_CHIₑᵣₐMut8 | 92 | Eubacterium ramulus |
| pET28b_CHIₑᵣₐMut9 | 93 | Eubacterium ramulus |
| pET28b_CHIₑᵣₐMut10 | 94 | Eubacterium ramulus |
| pET28b_CHIₑᵣₐMut11 | 95 | Eubacterium ramulus |
| pET28b_CHIₑᵣₐMut12 | 96 | Eubacterium ramulus |
| pET28b_CHIₑᵣₐMut13 | 97 | Eubacterium ramulus |
| pET28b_CHIₑᵣₐMut14 | 98 | Eubacterium ramulus |

The sequence SEQ ID NO 159 coding for SEQ ID NO 160 was synthesized and cloned into the pET28a vector between NdeI and XhoI restriction sites (Twist BioScience, San Francisco, USA) to obtain plasmid pET28a_his-AtDBR1.

By artificial design, certain mutants of AtDBR1 were created to optimize the activity and/or specificity of the AtDBR1 enzyme. These AtDBR1 mutant variants correspond to SEQ ID NOs: 161 to SEQ ID NO: 168 which code for SEQ ID Nos: 169 to SEQ ID NO: 176 and were generated via site-directed mutagenesis using the Q5® Site-Directed Mutagenesis Kit (New England Biolabs, Germany). For this, vector pET28a_his-AtDBR1 containing SEQ ID NO 159 was amplified by polymerase chain reaction (PCR) according to manufacturer's manual with one primer pair of same pair number from sequences SEQ ID NO 177 to SEQ ID NO 188, respectively. After digestion and ligation according to manufacturer's manual, the mixture was transformed into E. coli DH5α as described in Example 1 to obtain plasmids listed in Table 4.

TABLE 4
Obtained plasmids and organisms, the enzyme sequences are obtained of.

| Plasmid | Insert SEQ ID NO | Organism |
|---|---|---|
| pET28a_his-AtDBR1 | 2 | Arabidopsis thaliana |
| pET28a_his-AtDBR1_V285Q | 161 | Arabidopsis thaliana |
| pET28a_his-AtDBR1_V285T | 162 | Arabidopsis thaliana |
| pET28a_his-AtDBR1_V285D | 163 | Arabidopsis thaliana |
| pET28a_his-AtDBR1_V285L | 164 | Arabidopsis thaliana |
| pET28a_his-AtDBR1_Y81F | 165 | Arabidopsis thaliana |
| pET28a_his-AtDBR1_Y276A | 166 | Arabidopsis thaliana |
| pET28a_his-AtDBR1_Y290A | 167 | Arabidopsis thaliana |
| pET28a_his-AtDBR1_Y290F | 168 | Arabidopsis thaliana |

### 3. Cultivation of E. coli Cells and Biotransformation With Ene Reductases

E. coli BL21 (DE3) cells containing pCDFDuet-1CHI and one of the pET28a plasmids from Table 1 or E. coli BL21 (DE3) cells containing only one of the pET28a plasmids from Table 1 were used to inoculate 5 mL LB medium (Carl Roth GmbH, Karlsruhe, Germany) with the necessary antibiotics, respectively. After incubation of 16 h (37° C., 200 rpm), cells were used to inoculate 50 mL TB medium (Carl Roth GmbH, Karlsruhe, Germany) at OD₆₀₀ of 0.1 with necessary antibiotics. Cells were grown (37° C., 200 rpm) to OD₆₀₀ of 0.5-0.8 and 1 mM isopropyl-β-D-thiogalactopyranoside were added to the cultures. Cell cultures were incubated for 16 h (22° C., 200 rpm), centrifuged (10 min, 10,000 rpm) and supernatant discarded. The cell pellet was lysed using B-PER protein extraction reagent (Thermo Fisher Scientific, Bonn, Germany) according to manufacturer's instructions. After subsequent centrifugation (10 min, 20.000 rpm) the supernatant was used for biotransformations by addition of 1.5 mM nicotinamide adenine dinucleotide phosphate, 1.5 mM nicotinamide adenine dinucleotide, 1 M glucose, 1 U glucose dehydrogenase and 1 mM substrate. Butein, homobutein, 4-O-methyl butein, naringenin chalcone, hesperetin chalcone, eriodictyol chalcone and homoeriodictyol chalcone were used as substrate, respectively. The reaction mixture was incubated at 30° C. for 16 h. After stopping the reaction with methanol (1 volume reaction mixture+1 volume methanol), the sample was centrifuged (20 min, 20,000 rpm) and the supernatant used for LC and LC-MS analytics.

### 4. Purification of CHI and Biotransformation

E. coli BL21 (DE3) cells containing one of the pET28b plasmids from Table 2 or Table 3 were used to inoculate 1 L LB medium at OD of 0.1. Cells were incubated at 37° C., 250 rpm until the OD reached 0.4-0.6 and induced by supplementing with 0.1 mM IPTG. After protein expression at 28° C., 250 rpm for 16 h, the cells were harvested by centrifugation at 4,000×g for 15 min. The harvested cells were lysed with 0.5 mg/mL lysozyme (Sigma-Aldrich), 0.4 U/mL Benzonase (Sigma-Aldrich) and BugBuster (Merck) at room temperature for 0.5 h to obtain a crude cell extract. The crude extract was centrifuged at 10,000×g for 30 min to remove the pellet. Recombinant proteins were purified by Ni-affinity chromatography (GE Healthcare). The supernatant of the crude extract was loaded on the column with 20 mM imidazole. The column was washed by 5 column volumes of 30 mM imidazole with 20 mM PBS (pH 7.4) and 500 mM NaCl. The target proteins were eluted by 150 mM imidazole with 20 mM PBS (pH 7.4) and 500 mM NaCl. Buffer exchange with 50 mM PBS (pH 7.5) was achieved by ultrafiltration with Amicon Ultra-15 (Merck). The activity of the respective purified enzyme was determined by measuring the absorbance decrease at 384 nm in the reaction mix (CHI in 50 mM PBS with 100 μM of either naringenin chalcone, hesperetin chalcone, eriodictyol chalcone, homoeriodictyol chalcone or 4-O-methyl butein at 25° C.). The results are shown in table 5.

TABLE 5
Specific activity of potential bacterial CHIs and CHIₑᵣₐ mutants and their functional
amino acid substitutions.

| | | | | | | | | | | | Homo- | | |
|---|---|---|---|---|---|---|---|---|---|---|---|---|---|
| | | | | | | | | | Narin- | Erio- | erio- | Hesp- | |
| | | | | | | | | | genin | dictyol | dctiol | eretin | 4-O- |
| | Access | | | | | | | | chal- | chal- | chal- | chal- | methyl |
| Label | no. | 121 | 122 | 79 | 87 | 40 | 125 | 37 | cone | cone | cone | cone | butein |
| CHI1 | WP_ | N | T | I | L | Q | G | S | 21.3 ± | 18.2 ± | 11.3 ± | 16.7 ± | 0.2 ± |
| | 069876268.1 | | | | | | | | 1.8 | 2.0 | 1.6 | 0.7 | 0.0 |
| CHI2 | WP_ | G | D | P | K | Q | R | S | 54.8 ± | 25.4 ± | 19.9 ± | 93.5 ± | 0.8 ± |
| | 013779275.1 | | | | | | | | 3.1 | 3.1 | 5.2 | 4.5 | 0.3 |
| CHI3 | WP_ | G | D | P | K | Q | R | S | 20.0 ± | 13.2 ± | 7.8 ± | 12.8 ± | — |
| | 119972745.1 | | | | | | | | 0.9 | 1.2 | 0.3 | 2.9 | |
| CHI4 | WP_ | G | D | P | K | Q | R | S | 46.6 ± | 34.4 ± | 15.2 ± | 58.5 ± | 0.4 ± |
| | 079589066.1 | | | | | | | | 1.5 | 1.7 | 7.7 | 4.0 | 0.0 |
| CHI5 | WP_ | G | D | P | K | Q | R | S | 59.9 ± | 82.7 ± | 19.7 ± | 84.5 ± | 0.4 ± |
| | 073993359.1 | | | | | | | | 4.5 | 4.0 | 1.7 | 3.1 | 0.0 |
| CHI6 | HCD45524.1 | G | D | I | K | Q | R | S | 16.8 ± | 17.0 ± | 8.1 ± | 3.3 ± | 0.1 ± |
| | | | | | | | | | 0.5 | 1.0 | 1.9 | 0.2 | 0.0 |
| CHI7 | WP_ | G | N | D | K | Q | R | S | 68.3 ± | 123.6 ± | 50.2 ± | 6.4 ± | — |
| | 013978403.1 | | | | | | | | 3.3 | 12.3 | 9.3 | 0.4 | |
| CHI8 | SCJ10619.1 | G | D | P | N | K | R | S | 73.5 ± | 33.9 ± | 22.8 ± | 19.2 ± | 1.2 ± |
| | | | | | | | | | 4.5 | 2.0 | 0.7 | 0.9 | 0.1 |
| CHI9 | WP_ | G | E | P | N | K | R | S | 22.7 ± | 69.7 ± | 10.4 ± | 43.0 ± | 0.4 ± |
| | 118702262.1 | | | | | | | | 0.8 | 3.5 | 1.4 | 2.5 | 0.0 |
| CHI10 | WP_ | A | D | P | N | K | R | S | 0.7 ± | 2.5 ± | 0.2 ± | 1.2 ± | — |
| | 013977558.1 | | | | | | | | 0.0 | 1.3 | 0.0 | 0.0 | |
| CHI11 | SCK02381.1 | G | D | P | K | Q | R | S | 23.2 ± | 10.8 ± | 11.4 ± | 5.8 ± | — |
| | | | | | | | | | 1.0 | 1.0 | 0.8 | 0.2 | |
| CHI12 | WP_ | G | D | P | K | Q | R | S | 32.4 ± | 28.2 ± | 6 .2 ± | 36.5 ± | 0.2 ± |
| | 005036737.1 | | | | | | | | 4.0 | 1.5 | 1.3 | 0.8 | 0.0 |
| CHI13 | WP_ | G | D | E | N | K | R | S | 32.7 ± | 66.2 ± | 21.7 ± | 2.0 ± | — |
| | 125715151.1 | | | | | | | | 2.6 | 4.2 | 3.1 | 0.9 | |
| CHI14 | WP_ | G | D | E | N | K | R | S | 45.9 ± | 50.4 ± | 28.8 ± | 2.1 ± | — |
| | 026657636.1 | | | | | | | | 3.3 | 2.8 | 2.4 | 0.3 | |
| CHI15 | WP_ | G | D | E | R | T | R | S | 14.5 ± | 11.7 ± | 2.2 ± | 1.3 ± | — |
| | 095917646.1 | | | | | | | | 0.5 | 0.4 | 0.7 | 0.3 | |
| CHIₑᵣₐ | | G | N | D | K | Q | R | S | 270.0 ± | 90.7 ± | 44.5 ± | 31.5 ± | 0.1 ± |
| | | | | | | | | | 9.4 | 9.3 | 0.8 | 0.9 | 0.0 |
| CHIₑᵣₐ | | G | R | E | N | K | G | S | 32.6 ± | 59.5 ± | 23.7 ± | 93.2 ± | 0.4 ± |
| Mut1 | | | | | | | | | 4.0 | 2.4 | 1.7 | 1.5 | 0.0 |
| CHIₑᵣₐ | | G | R | P | K | Q | G | S | 4.4 ± | 8.3 ± | 3.5 ± | 11.5 ± | — |
| Mut2 | | | | | | | | | 0.5 | 0.2 | 0.8 | 0.3 | |
| CHIₑᵣₐ | | G | R | E | N | K | G | S | 16.7 ± | 39.2 ± | 13.0 ± | 55.1 ± | 0.3 ± |
| Mut3 | | | | | | | | | 0.2 | 1.0 | 3.9 | 4.9 | 0.0 |
| CHIₑᵣₐ | | G | R | E | K | Q | K | S | 2.3 ± | 8.9 ± | 4.8 ± | 4.7 ± | — |
| Mut4 | | | | | | | | | 1.6 | 0.5 | 3.4 | 1.2 | |
| CHIₑᵣₐ | | G | R | P | N | K | K | S | 8.0 ± | 13.5 ± | 8.8 ± | 4.5 ± | 0.1 ± |
| Mut5 | | | | | | | | | 0.7 | 0.3 | 2.5 | 0.7 | 0.0 |
| CHIₑᵣₐ | | G | D | E | R | N | R | S | 17.1 ± | 52.4 ± | 20.0 ± | 30.3 ± | 0.4 ± |
| Mut6 | | | | | | | | | 4.0 | 3.5 | 10.3 | 2.6 | 0.0 |
| CHIₑᵣₐ | | G | D | E | R | T | R | S | 61.4 ± | 181.3 ± | 62.2 ± | 28.4 ± | — |
| Mut7 | | | | | | | | | 3.3 | 6.0 | 1.6 | 0.6 | |
| CHIₑᵣₐ | | G | E | P | N | K | R | S | 25.6 ± | 49.0 ± | 21.2 ± | 83.9 ± | 0.5 ± |
| Mut8 | | | | | | | | | 2.3 | 5.0 | 9.0 | 5.4 | 0.0 |
| CHIₑᵣₐ | | G | N | M | K | Q | R | S | 67.5 ± | 43.7 ± | 14.4 ± | 57.4 ± | — |
| Mut9 | | | | | | | | | 3.1 | 1.3 | 0.3 | 3.8 | |
| CHIₑᵣₐ | | G | D | I | K | Q | R | S | 26.4 ± | 60.0 ± | 17.9 ± | 96.4 ± | — |
| Mut10 | | | | | | | | | 4.8 | 1.5 | 2.1 | 4.8 | |
| CHIₑᵣₐ | | G | D | P | K | Q | R | S | 76.8 ± | 91.1 ± | 30.8 ± | 75.5 ± | — |
| Mut11 | | | | | | | | | 4.9 | 7.1 | 1.5 | 4.8 | |
| CHIₑᵣₐ | | A | R | P | N | K | R | S | 11.0 ± | 19.9 ± | 9.8 ± | 24.9 ± | — |
| Mut12 | | | | | | | | | 1.3 | 0.5 | 1.7 | 0.5 | |
| CHIₑᵣₐ | | D | D | P | N | K | R | S | 28.0 ± | 28.0 ± | 33.6 ± | 107.1 ± | 1.4 ± |
| Mut13 | | | | | | | | | 3.6 | 0.9 | 5.3 | 9.7 | 0.1 |
| CHIₑᵣₐ | | H | R | E | N | K | R | S | 29.5 ± | 27.2 ± | 15.6 ± | 4.5 ± | — |
| Mut14 | | | | | | | | | 1.8 | 1.0 | 1.0 | 1.1 | |

### 5. Purification of AtDBR1 and Substrate Feeding

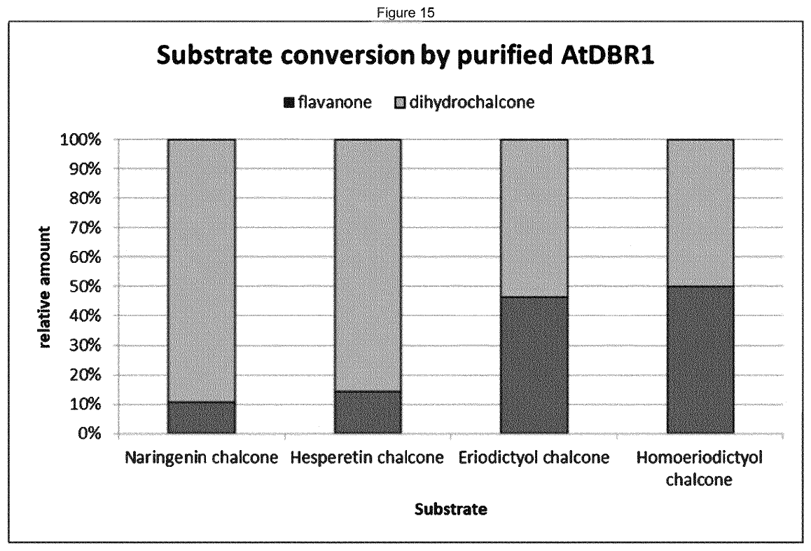

E. coli BL21 (DE3) cells containing plasmid pET28a_his-AtDBR1 were used to inoculate 5 mL LB medium (Carl Roth GmbH, Karlsruhe, Germany) with the necessary antibiotics. After incubation of 16 h (37° C., 200 rpm), cells were used to inoculate 50 mL TB medium (Carl Roth GmbH, Karlsruhe, Germany) at OD₆₀₀ of 0.1 with necessary antibiotics. Cells were grown (37° C., 200 rpm) to OD₆₀₀ of 0.5-0.8 and 1 mM isopropyl-β-D-thiogalactopyranoside were added to the cultures. Cell cultures were incubated for 16 h (22° C., 200 rpm), centrifuged (10 min, 10,000 rpm) and supernatant discarded. The cell pellet was lysed using B-PER protein extraction reagent (Thermo Fisher Scientific, Bonn, Germany) according to manufacturer's instructions. After subsequent centrifugation (10 min, 20,000 rpm) the supernatant was purified using a 1 mL HisTrap FF column (GE Healthcare) following the manufacturers manual. Eluted protein was desalted using a PD-10 desalting column (GE Healthcare) using the gravimetric protocol of manufacturer's manual. Protein was eluted into 50 mM phosphate buffer pH 6.0. Purified protein was used for biotransformations by addition of 1.5 mM nicotinamide adenine dinucleotide phosphate and feeding of substrate (naringenin chalcone, hesperetin chalcone, eriodictyol chalcone or homoeriodictyol chalcone were used, respectively). The reaction was supplemented with 10 ppm of substrate every 10 min for 150 min. Incubation was performed at 30° C. After stopping the reaction with methanol (1 volume reaction mixture+1 volume methanol), the sample was centrifuged (20 min, 20.000 rpm) and the supernatant used for LC and LC-MS analytics (see FIG. 15).

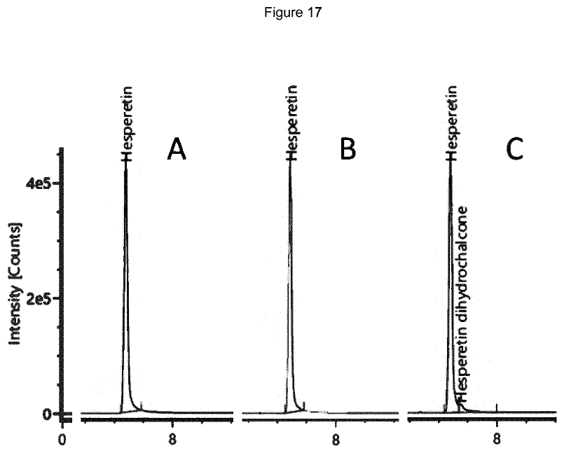

Purified AtDBR1 was used for biotransformations by addition of 1.5 mM nicotinamide adenine dinucleotide phosphate and 10 μM hesperetin. A control reaction was performed as stated above but with 50 mM phosphate buffer pH 6.0 instead of purified AtDBR1. Reactions were incubated at 30° C. Reactions were stopped with methanol (1 volume reaction mixture+1 volume methanol) after 0 min of incubation or after 90 min of incubation. Samples were centrifuged (20 min, 20,000 rpm) and the supernatant used for LC-MS analytics. The results are depicted in FIG. 17.

### 6. Cultivation of E. coli Cells and Biotransformation With AtDBR1 Variants

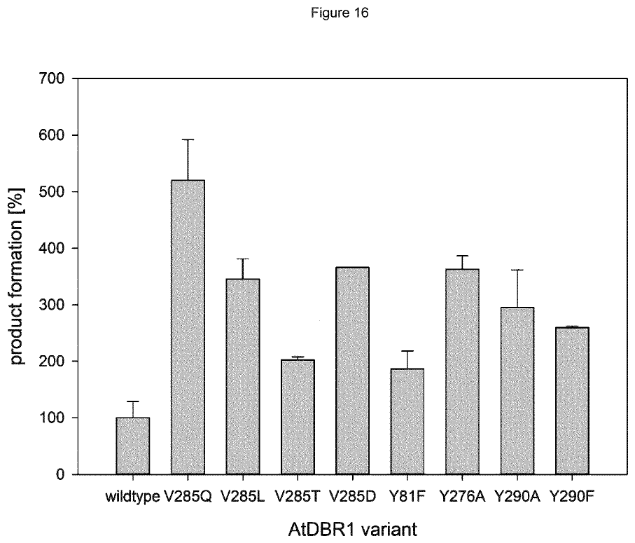

E. coli BL21(DE3) cells containing one of the pET28a plasmids from Table 4 were used to inoculate precultures of 450 μL LB medium (Carl Roth GmbH, Karlsruhe, Germany) with the necessary antibiotics, respectively. After incubation of 6 h (37° C., 300 rpm), cells were used to inoculate 665 μL TB medium (Carl Roth GmbH, Karlsruhe, Germany) with necessary antibiotics with 35 μL of the respective preculture. Cells were grown (37° C., 300 rpm) for 75 min and 50 μl 15 mM isopropyl-β-D-thiogalactopyranoside were added to the cultures. Cell cultures were incubated for 16 h (28° C., 300 rpm), centrifuged (20 min, 5,000 rpm) and supernatant discarded. The cell pellet was lysed in 500 μL B-PER protein extraction reagent (Thermo Fisher Scientific, Bonn, Germany) according to manufacturer's instructions. After subsequent centrifugation (60 min, 5,000 rpm), the supernatant was used for biotransformations by addition of 1.5 mM nicotinamide adenine dinucleotide phosphate and 20 μM of substrate hesperetin chalcone. The reaction mixture was incubated at 40° C. for 3.5 h. After stopping the reaction with methanol (1 volume reaction mixture+1 volume methanol), the sample was centrifuged (60 min, 5,000 rpm) and the supernatant used for LC analytics. The results are depicted in FIG. 16.

1. Method for the biocatalytical manufacturing of dihydrochalcones, comprising or consisting of the steps:
i) providing at least one ene reductase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 24 to 46 and 169 to 176;
ii) optionally providing at least one genetically engineered chalcone isomerase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 145 to 158;
iii) providing at least one flavanone and/or at least one chalcone and/or at least one of the corresponding glycosides;
iv) incubating the at least one ene reductase provided in step i) and optionally the at least one chalcone isomerase provided in step ii) together with the at least one flavanone and/or the at least one chalcone and/or the at least one corresponding glycoside provided in step iii);
v) obtaining at least one dihydrochalcone;
vi) optionally purifying the obtained dihydrochalcone.

2. Method according to claim 1, wherein the at least one ene reductase provided in step i) is purified or partially purified.

3. Method according to any one of claim 1 or 2, wherein the incubation in step iv) is done for at least 5, 10, 15, 20, 25 minutes, preferably for at least 30 minutes.

4. Method according to any one of the previous claims, wherein the at least one flavanone and/or at least one chalcone and/or at least one of the corresponding glycosides provided in step iii) is selected from the group consisting of homoeriodictyol, hesperidin, hesperetin-7-glucosid, neohesperidin, naringenin, naringin, narirutin, liquiritigenin, pinocembrin, steppogenin, scuteamoenin, dihydroechiodinin, ponciretin, sakuranetin, isosakuranetin, 4,7-dihydroxy-flavanon, 4,7-dihydroxy-3′-methoxyflavanon, 3,7-dihydroxy-4′-methoxyflavanon, 3′4,7-trihydroxyflavanon, alpinentin, pinostrobin, 7-hydroxyflavanon, 4′-hydroxyflavanon, 3-hydroxyflavanon, tsugafolin.

5. Method according to any one of the previous claims, wherein at least one flavanone and/or at least one chalcone and/or at least one of the corresponding glycosides is provided in step iii), and wherein the at least one flavanone and/or at least one chalcone and/or at least one of the corresponding glycosides is additionally purified or partially purified.

6. Method according to any one of the previous claims, wherein the at least one dihydrochalcone obtained in step v) is/are selected from the group consisting of butein dihydrochalcone, homobutein dihydrochalcone, 4-O-methylbutein dihydrochalcone, naringenin dihydrochalcone, hesperetin dihydrochalcone, homoeriodictyol dihydrochalcone and eriodictyol dihydrochalcone.

7. Genetically engineered ene reductase comprising or consisting of an amino acid sequence having at least 90%, 91%, 92%, 93%, 94%, 95%, 96%, 97%, 98%, 99% or 100% sequence homology to a sequence selected from the group consisting of SEQ ID NOs 169 to 176.

8. Transgenic microorganism comprising a nucleic acid sequence encoding a genetically engineered ene reductase according to claim 7.

9. Transgenic microorganism according to claim 7 or 8, wherein the microorganism is selected from the group consisting of Escherichia coli spp., such as E. coli BL21, E. coli MG1655, preferably E. coli W3110, Bacillus spp., such as Bacillus licheniformis, Bacillus subitilis, or Bacillus amyloliquefaciens, Saccharomyces spp., preferably S. cerevesiae, Hansenula or Komagataella spp., such as. K. phaffii and H. polymorpha, preferably K. phaffii, Yarrowia spp. such as Y. lipolytica, Kluyveromyces spp, such as K. lactis.

10. A vector, preferably a plasmid vector, comprising
at least one nucleic acid sequence encoding an ene reductase having at least 90%, 91%, 92%, 93%, 94%, 95%, 96%, 97%, 98%, 99% or 100% sequence homology to an amino acid sequence selected from the group consisting of SEQ ID NOs 24 to 46 and 169 to 176,
and optionally
at least one nucleic acid sequence encoding a genetically engineered chalcone isomerase having at least 90%, 91%, 92%, 93%, 94%, 95%, 96%, 97%, 98%, 99% or 100% sequence homology to a sequence selected from the group consisting of SEQ ID NOs 145 to 158.

11. Use of at least one ene reductase according to claim 7 and/or at least one transgenic microorganism according to claim 8 or 9 and/or at least one vector according to claim 10, in the biocatalytical manufacturing of dihydrochalcones, preferably in a method according to any one of claims 1 to 6.


SEQUENCE LISTING (190 sequences)

SEQ ID NO: 1 - DNA, 1041 bp, Arabidopsis alpina
atgggcaccgcaaccaacaaacaggtcattttcaaagattatgtgagcggcttcccgaaagaatcagactttgatttcaccacgactaccgttgagctccgcgtccctgaaggaagcaatagtgtgctggttaaaaatctgtatttatcgtgtgacccatacatgcgcatccgcatggggaaacctgatccagaaaccgccgcattagccgcggcatatcctgtcggtcagccgattattggctatggcgttgcccgcgtcatcgaatcggcgcacccagactacaaaaagggcgacctgttatggggtttcgttgggtgggaagaatattcacttttcaccccggtgcacaatttacatttcaagatccatcataccgatgtgcctctctcctactatacgggtttattgggtatgccgggtatgacggcctacgccggcttttatgagatttgtagtcctaaaaaaggtgaaaccgtctatgtgtctgctgcctcgggtgcggtgggtcagctggtgggccagttcgcgaaaatgatgggctgctacgttgttggctcggcgggttcgaaagaaaaagtggaccttctgaagacgaaatttggattcgatgatgcctttaactacaaagaagaacaggacttgaccgcggcattgaaacgttgcttcccgaagggtatcgatatttatttcgaaaacgtcggcggcaaaatgctggatgccgtcctgctgaacatgaatccgcacggtcgtatcgctgtgtgtggtatgatttcgcagtacaacctgcaggatcaggagggagttcacaacctgagctcaattatttacaagcgtattcgtatccagggcttcgtggtgtccgatttttacgataaatatagtaaattcttggaattcgtagtgccgtgtattaaagaaggcaaaatcacatacgtcgaggacgttgcggagggtctggaaaaagggccggaagcactcgtgggtctgttccatggtaaaaatgtaggaaaacaagtagtcgtggtggctcgggaatga

SEQ ID NO: 2 - DNA, 1038 bp, Arabidopsis thaliana
atgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttgtagtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 3 - DNA, 1041 bp, Brassica cretica
atgggcgcatcgaacaacaagcaggtgattctgcgcgattatgttgcgggtttcccgaaagagtcagatctggtattcaatgatgctaccgtggatctgtctgttccggcaggctcaaacaaagttctggtcaaaaatctgttccttagctgtgatccgtatatgcgtatccgcatgtcgaaaccggatccgtcgactgcggctctggccttgccgtataagcccggacagcctatttacggctacggagtcagtaaggtaattgaatcggggcatcctgattatacgaaaggtgatctgctgtgggggattgtaggctgggaggagtattcggtgatcacgctgacgccatacagtcattttaaaatcctgcacaccgatgtgcccctttcctactacaccggattactgggcatggcaggcatgactgcttatagtggattctatgagatctgctctccgaaaaaaggagaaacagtgtttgtatcggctgcaagcggcgccgttggccagttggtgggccaattagcaaagatcatggggtgttacgtggtgggcagtgcggggtccaacgaaaaagtggaactgctgaagaacaaatttggcttcgatgaggcattcaactataaagaggagcaagatctgaatgcggcactgaagcgctgtttccccgaaggtattgacatttatttcgagaatgttggcggtaaaatgctggacgccgttttgctgaacatgaagctgaatggccgcattgcagtatgtggaatgatttcccagtataaccttgaggaacaagagggtgttcgcaatctggctacagtcatttataaacgcgttcgcctgcaggggttcgtggtgagtgactattaccacaagtactcaaatttcctggaattcgtgcttccgtacatccgcgaaggtaaaatcacctatgttgaagatgttgcagaaggtttggagaatgggccgtcagcgctgatcggcttatttcatggcaaaaacgtcggtaaacagctcatcgttgtggctcgggaataa

SEQ ID NO: 4 - DNA, 1002 bp, Brassica rapa
atgggcgcaaccaataaacaagttattctgcgcgattatgtctcaggcttccctaaagaatctgacttcgagattacgacaacgacggtcgaattgcgtttgccggaagggtccaactctgttctggtaaaaaatctgtacttgtcctgtgacccatacatgcgctcgcgcatgggcaaaccctccgcgttcgctttggcccaggcgtttacgccgggtaagcctattctcggttttggcgtcagtcgtgtgattgaaagtggccatccgggatacaaacaaggcgatttgttgtggggtattgtaggctgggaagaatattccgtggttaccccagaaatgcactttaaaatccacgacaccgatgtcccgttgtcttactacaccggcctgctgggcatgccggggatgaccgcgtatgcaggcttccacgaggtctgctcaccgaaaaaaggggagaccgtgtatgtgtctgctgcgagtggtgcagtaggtcagctcgtaggtcagtttgcgaaaatgatgggttgctatgtggttggtagcgcgggctccaaagagaaagtcgatcttctgaagaacaaatttggatttgacgacgcgtttaattacaaagaagaacaggacctgtccgccgcgcttaaacgttgcttcccgaaaggtatcgatatctacttcgaaaacgtgggcggtaaaatgctggatgcggtactcctgaacatgaacatgcacggccgtattgctgtgtgcgggatgatctcgcaatataatctcgaagatcaggaaggagttcataacttgacttccatcatctataaacgcattcgcattcagggattcgtggttttcgattatttcgaaaaatattccaactttatggaatttgtgatcccttgcattaaagaagagaaaattgcttatgtggaagacgtggccgagggactggaaaaaggcccggaagcgctggttggtctgttctacggtaaaaacgtcggaaagtga

SEQ ID NO: 5 - DNA, 990 bp, Cavia porcellus
atggtaaaggcgaaatcgtggaccctgaaaaaacactttcagggcaaacccacccagagcgactttgaattgaaaactgtcgaactgccaccgttgaaaaacggtgaagtactgctggaggcactctttctgagcgtggacccgtatatgcgtatcgcctctaagcgtctgaaagaaggtgcggtgatgatggggcagcaggtggctcgtgtcgttgaatccaaaaacagtgcctttccggccggctccattgtgttagcgcagtctgggtggaccactcactttatcagcgatggcaagggcttggaaaaacttctgaccgagtggccggacaaacttccgctgtctttggcgttaggaacgattggtatgcctggtctcaccgcttattttggtctgctggaagtgtgcggggtaaaagggggcgaaaccgtgctcgttagcgctgccgcaggtgcagtcggctcggttgtcggccaaattgcgaagcttaaggggtgcaaagtagtgggtgcggccggctcggacgagaaaatcgcgtatctgaagcaaattggtttcgacgccgcgtttaattacaagacggtaaattcgctggaggaagccttaaaaaaagcttccccggatggatatgactgctatttcgataacgtagggggtgaattccttaataccgtgctttcgcagatgaaggattttggcaaaattgcgatttgcggcgcaatctcggtttataaccgtatggatcagctgccgccgggtccaagcccggaatctatcatctacaaacaattacgtatcgaaggcttcatcgtttatcgctggcagggcgatgttcgcgaaaaagctcttcgcgatttgatgaaatgggtgttagagggcaaaatccaatatcacgaacatgtcacaaaaggttttgagaatatgccagcggcgttcattgagatgttgaatggtgccaacttgggcaaggctgttgtcacagcctaa

SEQ ID NO: 6 - DNA, 1038 bp, Capsella rubella
atgggcacggcgacaaacaagcaggtcaccctgaaggattatgtgtccggtttcccaaaagagagcgattttgattttacgactaccaccgtcgaattgcgcgtgccggaaggttcgaactcagtactggtgaaaaatctgtatctgagttgtgatccatacatgcggactcgtatgagcaagccgaaaccatcaacggcactggcgcaggcctatgctttgggccaaccgatctttggtttcggtgttagccgggtgattgaaagcgggcaccccgattataagaaaggcgacttgctgtggggtatcgtgggctgggaagaatactctgtcattaccccaacgccggatgtgcattttaaaatccagcataccgatgtgcccctgagttactacactggcctgctcggcatgccgggcatgacggcctatgcggggttttacgagatttgtagcccgaaagagggtgaaactgtgtatgtctctgccgcgtcgggcgcagtaggtcaattggtgggacagtttgctaagatgatgggctgttacgtggtgggttctgcaggtagtaaagagaaagtcgatttactgaaaacgaaatttgggtttgatgatgcgtttaactacaaagaggaacccgacttgagcgccgcactgaaacggtgttttccgaaaggtattgatatctattttgaaaatgtgggggggaaaatgttagatgcggtactgcttaacatgaacgttcacgggcgcattgcagtatgcggtatgatttctcagtataatctggaaaatcaggagggggtacataacttaagcaatattatttataaacgcattcgcatccaaggatttgcggttttcgatttttatgataaatacagtaaatttttagaatttgtgctgccgcacatcaaggaaggcaagattacctacgtggaggacgttgcggatggcctggaaaaaggcccggaggctttagtcggcctgttccatggaaagaacgtgggcaaacaagttgtagttgtagcgcgggaatga

SEQ ID NO: 7 - DNA, 1038 bp, Capsella rubella
atgggcaccgctaccaacaagcaggtaaccctgaaagattatgtctctggttttccgaaggaaagcgatttcgatttcaccacaactaccgtggagctccgcgtacctgaaggctctaattctgttctcgtcaagaacttgtatttgagctgtgatccttacatgcgcacgcgtatgtcaaaaccgaagcctagtacggctcttgcgcaggcctatgcgctgggtcaaccgatcaacggttatggtgtgtctcgtgtaattgagtcgggtcatccggattataaaaaaggtgatttgttatggggtttcgtaggctgggaagaatatagtgtgattacgcctatgaaccgtatgcatttcaaaattcagcatacagatgttccgctttcatactataccggtctgctcggcatgccgggtatgactgcttacgccggcttctatgaaatttgttcgccgaaagagggcgaaacggtgtatgtgagcgctgcgtcgggtgcggttggccagttggtaggacagttcgccaaaatgatgggctgttatgtggtaggatcagctggttcgaaggagaaggtggatcttttgaaaaccaaattcggtttcgatgatgctttcaactataaagaggaacaagatctttctgcagctctgaagcgttgctttccgaaaggcattgacatctatttcgaaaatgtgggcggcaagatgctggatgctgtgctcttaaatatgaacccacatggtcgcattgccgtatgcggaatgatttcgcagtataatttggagaaccaagagggcgtgcataacttatcaaatattatttacaaacgcatccgcattcagggtttcgtggtgagcgacttctatgataaaatccccaaattcctggagtttgttttgccgcagatcaaagaaggtaaaatcacctacgtcgaggatgttgcagaaggtatcgaaaaaggcccagaagcgctgattggcctgtttcacggcaaaaacgtcggcaaacaggtcgtggtgatcgcccgtgagtaa

SEQ ID NO: 8 - DNA, 1041 bp, Camelina sativa
atgggcacggccactaacaataaacaggtgaccctgcgtgattatgtgagcgggttcccaaaggaatctgatttcgattttaccacctcgacggtggagctgcgcgtcccggaaggttcaaactcggttctggtcaaaaacctgtatctgagttgcgacccatatatgcgcacccgcatgggcgagccgaaaccgagcacggcacttgcacaggcttataccctgggcaagccaatttacggatatggtgtgagtcgtgttatcgagtcaggtcacccggattacaaaaaaggcgacttactgtggggtattgtaggctgggaggaatacagcgtgattacgccggcgccgcacatgcactttaaaatccaacataccgatgtaccactgagctattacactggccttctgggtatgccgggcatgacggcctatgcgggcttttacgagatctgcagcccgaaagaaggcgagactgtgtatgtttctgcagcctcaggcgcggttggccagctcgtgggtcagtttgcgaaaatgatgggttgttacgtagtgggcagcgccggctccaaagataaagttgatctgttgaaaaccaaattcggctttgatgatgcgtttaactacaaagaggaaccggatctgtctgccgcgttaaagcgctgttttccgaaagggatcgatatttactttgagaacgtcggtggtaaaatgctggatgcagtcctgcttaacatgaatgtccatgggcgtattgcggtgtgcgggatgatcagccagtataatcttgaaaatcaggaaggcgttcataacctgagcaacattatttataaacgcatccgtatccagggctttgccgtcttcgacttttacgacaaatacagcaaatttttggaattcgtactgccccacattaaagaaggtaaaattacgtatattgaagatgtcgctgatggtctggaaaagggcccggaggcccttgtcggcctcttccacggaaaaaacgtgggtaaacaggttgtagttgtggcacgtgagtga

SEQ ID NO: 9 - DNA, 1035 bp, Eutrema salsugineum
atgggcacagcaacaaacaaacaggtgattctgcgtgattacgttactggttttcctaaggaatcagacttcgattttactacgaccacggtagaattgcgtgtcccggaagggtctaactcagtgctggtcaaaaacctgtacttatcgtgcgacccctatatgcgcagccgcatgggaaagccggcagacagcgcgttggcacaggcgttcacggttggtcagccgattttgggatatggtgtcagtcgtgtaatcgagagtgggcacccggattacaaaaagggcgatttgctgtggggtattgttgggtgggaagaatactctgtaattaccccgatccccgacatgcactttaaaattcaccacaccgatgtaccgttgtcctattacaccggtttactcggaatgccgggcatgacagcgtatgccggtttctatgagatttgctccccgaaaaagggggagacggtgtatgtttccgctgcttcaggtgcggtcggccaactggtgggtcagttcgcgaaaatgatgggttgttacgtcgtgggatcggcgggttccaaagaaaaagtggacctgctgaaaacgaaattcggctttgatgatgcgttcaattataaagaggaacaggatctgtcagctgcactgaaacgttgtttcccgaagggtattgacatctactttgaaaacgttggtggcaaaatgctggacgcagtattgctgaacatgaatatgcatggccgtattgcggtctgcggcatgattagtcagtataacctggaagatcaagaaggtgtgcataaccttagtaccatcatttacaagcggattcgtatccagggcttcgttgtttatgatcactacgacaaatacagtaaatttttagaatttgttttaccgtgtatcaaagaagggaagattgcctatgttgaagacgtcgcggaaggcctcgagaaaggtccggaggcgctggtcggtctgtttcatgggaaaaatgtcgggaaacaagttgtggtagtagctcgggaataa

SEQ ID NO: 10 - DNA, 1041 bp, Eutrema salsugineum
atgggcacagtgacgaacaaacagatcatttttccggactacgtttccggctttccgaaagaatccgacttaaaaatcaccaccaacaccgtagacctgcgcctgccagaaggatccaaaagcgtgttagtgaaaaacctgtatctgagttgcgatccgtatatgcgcatttgcatggggaaaccggaccccttgacctctagcctcgttccgccgtacaacacaggtgagccgattgtcggcctgggcgtgagtaaagttattgactccggccatccggattacaaaaaaggcgacctgttatggggtctggttggttgggaggaatacacggtcattaccctgacgccgtacagtcattttaaaattcaccacacggatgtaccactgagctactacacgggcctccttggaatgcctggaatgaccgcatatgcaggcttttacgaaatctgcagccctaagaaaggcgaaacagtgtttgtttcggccgcgtccggcgctgtcggccagctcgttggccaatttgcgaaactcatgggctgctatgtcgtcggcagcgttggcagcaaagaaaaggttgatcttctgaagacgaaatttggtttcgacgatgcctttaattataaagaggaacaggatctgaacgccgctttaaaacgctgctttcccgaaggtatcgacatttactttgaaaacgttggtggtaaaatgttggacgctgtgctgattaacatgaagctccatggtcgcatcgcggtgtgtgggatgatcagccaatataaccttgaagacccggagggcgtacataacctggccacgatcctgtataagcgtattaaactgcagggatttggcgttttcgatttctatgacaaatattacaatttcctggattttgtgttgccctacatccgtgaagggaaaatcacttatattgaagatatcgcggaaggccttgaatcgggcccggccgcgctgttgggcttattccatgggaaaaatttcggcaaacaactgtttgttgttgctcgtgaatga

SEQ ID NO: 11 - DNA, 1050 bp, Homo sapiens
atgcaccatcaccaccaccattcaagcggcgtggatctgggtaccgaaaacctgtattttcaatctatgacgaagacctggacccttaaaaaacacttcgtgggttacccgactaacagtgacttcgaattaaaaaccagcgaactgccgccgctcaaaaacggggaggtgctgctggaagcactgtttttaacggtggacccctacatgcgcgttgcagcaaaacgcctcaaagagggtgatacgatgatgggtcaacaggtggcgaaagttgtcgaatccaaaaacgtggcacttccgaaagggacgattgtgttagccagcccggggtggacaactcacagtatcagtgatggaaaagatttggaaaagcttttaactgaatggccagatactattccgcttagtctcgcgttgggcaccgtgggtatgccgggcttaacagcttattttggtctgctggagatctgcggggttaaaggtggtgaaacggttatggttaacgcggcagccggcgctgtagggagtgtagtgggccagattgctaagctgaaaggttgcaaagtggtgggtgctgtgggttccgatgaaaaagtggcttaccttcaaaagctgggattcgatgtggtgttcaattataaaaccgtcgaaagcctcgaggaaaccctgaaaaaagcaagtccggatggttacgattgctacttcgacaacgtcggcggcgaattctccaacacggtgatcggccaaatgaaaaagttcggccgcattgctatctgcggcgcgatttccacatataaccgcacgggaccgctgccgcccgggccgcctccggaaattgttatctaccaggaactgcgtatggaagccttcgtggtctaccgttggcagggcgacgctcgtcagaaagcactgaaagatctgttaaaatgggtgctcgagggtaaaattcaatataaagaatatattattgaaggtttcgaaaatatgccggccgcgttcatgggaatgctgaaaggcgacaatttaggtaaaactatcgttaaggcttga

SEQ ID NO: 12 - DNA, 1035 bp, Microthlaspi erraticum
atgggcaccgccactaacaaacaaattattttgcgggattatgtatccggttttccgaaggagtcagactttgagttcaccaccacaaccgtggagctgcgggcgccggaaggctcaaaatccctgctggtgaaaaacctgtatctgtcctgtgacccgtatatgcgctcgcgtatgggaaaaccggctgcctcgggaatcgctcaggcgtttacggtgggcaaaccgattatcggattcggtgtgtcgcgcgtgattgaaagcggtcacccggactacaaaaaaggtgatctgatctggggaatcgtcggctgggaagagtattcggttgttacgccgatccctgatatgcactttaaaattcatgatacggatgttccattaagttactacactggcctccttgggatgccggggatgaccgcatatgcaggcttctacgaaatctgcagcccaaaaaaaggagaaaccgtttacgtcagcgctgcatctggcgcggtcgggcagctcgtgggccagtttgccaaaatgatgggctgctacgtggtaggtagcgccggtagccaggaaaaggtagacctgttaaaaacaaaattcggcttcgacgacgcttttaattacaaagaagaacaagatttaagcgcggctctgaaacgttgctttccgaaaggcattgatatttattttgaaaacgtcggtgggaaaatgcttgatgcagttttgttaaacatgaacacattcggacgggtggctgtttgcggaatgatcagtcagtataaccttgaagatcacgagggggtttataaccttacttctattatctataaacgtatccgtattcagggctttgcggtctttgatcactatgacaaatattccaaattcttagaactggtgatcccgagtatcaaggaagggaaaattgcctatgttgaagatgtcgcggaaggacttgaaaaagccccggaagccctggtcggtctgtttcacggcaaaaatgtaggcaaacaggttgtcgtggttgcgcgcgaataa

SEQ ID NO: 13 - DNA, 1041 bp, Microthlaspi erraticum
atgggcaccactaccaacaaacagattatttttcctgaatatgtttcgggatttccgaaagaaagcgatctcaatattaccacaacgactatcgacctgaaactccctgagggctctaccagtgttctcgtgaaaaatctgtatctgagctgcgacccgtatatgcgtatttgcatgggtaaaccggatccgctggcaaattccctggtgcccccatacattaccggcgaacctatcgtgggcttaggcgtctcgaaagtcatcgattctggtcatccggattataaaaaaggggatttactgtggggccttgttggctgggaagagtataccgtgattacgttgaccccatactctcatttcaaaattaaccataccgacgttcctttatcatattataccggtctgctgggtatgcctggtatgaccgcttacgcgggcttttatgaagtctgctctccgaaaaaaggtgagacggtctttgtgtcggcagcgagtggcgccgtgggccaactggtaggccagttcgcgaaaatgatgggctgttatgtggtggggtccgctggctccaaggaaaaagtggacctgctcaaagagaagtttggcttcgatgaggcgttcaattataaagaagaacaggatttaagcagtagcctgaaacgttgttttccggaaggcatcgatatctactttgaaaacgttggaggcaaaatgttagacgccgtactggttaacatgaaatcgcagggccgcattgcggtctgcgggatgatttctcagtacaacctggtcgataccgaaggtgtgcataatctgccgaccatcctgtataaacgcattaaaatccagggctttcgtgtgttcgatttctatgataagtattctaaatttttggacttcgtactgccgcacattcgtgagggcaacatcacgtatatcgaggatatcaccgaaggcttagaatccgggccgtctgcgttactcggcctctttcatggtaaaaacgtgggcaaacagttattcgttgtggcccgtgagtaa

SEQ ID NO: 14 - DNA, 1032 bp, Nicotiana tabacum
atggcggaagaagtttctaacaagcaagttatcctgaagaactatgtcaccggttatccgaaagaatctgatatggaaatcaagaatgttactattaaattaaaagtcccagaagggtccaacgatgttgtggttaagaatctgtatttgtcgtgcgacccatatatgcgctcacgtatgcgtaaaatcgaaggcagctatgtcgagtcttttgcgccggggtcgcctatcacaggctatggagtagccaaagtgctggagagcggcgacccgaaattccagaaaggcgatttggtgtggggtatgactggatgggaagaatactcgattatcacgccgacccagaccctgttcaaaattcacgataaggatgtgccactgagttactataccggcattctgggtatgccgggtatgacggcatatgccggctttcacgaggtatgttcgcctaaaaaaggtgaaactgtcttcgtatcggctgcgagcggcgcggtgggccagttagtggggcaattcgccaaaatgctgggctgctatgtcgtggggtcagcggggtccaaagagaaagtggacctcctcaaaagcaaattcgggtttgatgaggccttcaactataaagaggaacaggatctttcggccgcattaaaacgttactttccggatggcatcgatatttatttcgaaaacgtaggaggcaaaatgttggacgcggtacttgtcaacatgaaactttacggtcgtattgcggtctgtggcatgatttctcagtataacctggaacaaaccgaaggcgtccataacttgttctgtctgattacgaaacgcatccgtatggaaggctttttagtgtttgactattaccacctttacccaaagtatctggaaatggtgatcccgcagattaaagccggcaaggtagtgtatgtagaagatgttgctcatggtctggagagcgcgccgaccgccctggtgggtctgttttccggtcgtaacatcgggaagcaggtggtgatggtctctcgtgaataa

SEQ ID NO: 15 - DNA, 1038 bp, Olimarabidopsis pumila
atgggcacggcgaccaacaaacaggtgattctgaaagattatgtgagcggctttccgaaggaatcggactttgatttcaccacaaccaccgttgagttaaaagtaccggagggttcaaatagcgtgcttgtcaaaaacctgtacttaagctgtgatccttacatgcgcacccgcatgggcaagccgaaaccttcaacggcactggctcaagcgtatgctctgggccagccgatctacggctatggcgtaagtcgtgtaatcgaaagtggtcatccggattacaaaaaaggcgacctgttatggggtattgtgggctgggaagaatattcggtaatcaccccgaccccggatgtgcactttaaaattcaacacacgcacgtcccgctcagctattatacgggtctgcttggcatgccgggcatgacggcctatgcaggcttctatgaaatttgctctcccaaagaaggcgagactgtgtacgtatctgcggcaagtggcgcggtcgggcagttagtgggtcagttcgctaaaatgatgggttgttatgtggtcggcagcgcggggtccaaggagaaagttgacctgctgaaaaccaaatttggctttgacgacgcttttaattataaagaagagcctgatttgtctgcagcactcaaacgttgctttccgaaaggtgtcgatatttattttgaaaacgttggcgggaagatgctggatgcggtcctgttaaacatgaatgtccacggccgtattgcggtgtgtggaatgattagccagtacaacttagagaaccaggaaggtgttcacaatcttagtaacattatttacaaacgtattcgtattcagggatttgctgtttttgatttctatgataaatatagtaagtttttagagtttgtactgccgcagatcaaagaaggtaaaattaagtatgttgaggatgttgcagatggactcgagaaaggcccggaagcactggtaggcctgtttcacggcaaaaacgtaggaaagcaagtggtggtgatcgcccgtgaatga

SEQ ID NO: 16 - DNA, 1026 bp, Plagiochasma appendiculatum
atggcgggcacggaagtcaccaaccgccaaattctgttcaaaaatttcgtggttgggtggccgcaggaaagtgatatggaatttgtaaccacgaaaaaaaaattggaactccgtgaagggcagaaagacgtgattgttcgcgttctgtacctgagctgtgatccgtaccagcgcggtcgtatgcgtaatagcacagattcttatattccgccgtataccccggggaagccgatcgaaggttatggtctgggcaaagtaattttgtctagcgatccggcattcaacgaaggagacattgtgtccggtctgatgggttgggaagattattcgtatggctttcgtctggtcaaaattgatgacctggcactgccgatctcctattacctgggtgcgctgggaatggcggggtttacagcctatgtgggattttttcgcatttgtgaaccgaagaagggcgatacgatctatgtaagcgcagcgtcgggcgccgtcggacagatggtaggtcaatttgcaaaactgtttggatgccgcgtggttggttctgcggggagtcaacagaaagtcgatttactcacctcgaaattcggctatgatgaagcctttaactacaaagaggagccggacctggatgcggcgctcaaacgttgttgcccggatggaattgatatctacttcgaaaatgtcgggggtaaaatgctggatgcggtgctggtcaacatgaaggtctttgggcgcatcgcggtctgtggcatggtttcgcagtataacaaagaagtacaagaccccatttacaatcttaactatacggtgaaacggcgtctgaaaattcacggcttcctgcagtccgaccatatggatgtgcagcccgaattttttaaaaacgtgatccagtggtttaaggaaggtaaacttgtgtatgtggaagatattgcagatggtctggaaaaaggccctgccgcactcattggtctccttgctggaaagaatgtggggaaacaaagcgtgaaaatcgccgacccctga

SEQ ID NO: 17 - DNA, 1056 bp, Pinus taeda
atggaacaacgggtgcccaaccgcgagctgattcttgtggcatatgcaaacgaaggcccggtgaccgattctcatctgaacattcgtgaaactaaacttgatttaggtagtgtgggaaaggacggcagctcgggggacgtggcggtccagaatctgtggatttcggtagatccctacctgcggcagctgatgaaagaaagtgacgacggactgtatctgccatcgtttccgcttaatcaagctatccgttctattctggttggtaaagtcgttgcctcggctaaccccgcattcgaagttggtgacattgtaagcggtttctaccaggtaagtgaatacgcgattgttccacgtggtgatctcatgaaaatcgacacaagtgtcgtgaaaccgagtgactatctggggttactgggtatgcctgcactgacagcatgggcggggttcattattgtcggcgaacctaagccgggggacgaagtcttcgttagcgcggccgccggttctgtaggcatgctggtgggccagcttgcgaagattaaatcttgtcgcgttgtgggttccgcgggcagtgatcagaaagtaaagctgctcaaagagtttggctttgatgatgcttttaactacaaatgcgaaaccgatctggacgctgcgctgagccggtacttcccccgcggtatcgacatctattttgataacgtaggcggtcgtatgctggaagcggtgctgaatcatatcaacatgaaagcgcgcattccgttatgtggtatgattagccagtataaccaagaatggaaacagcgtttcggtgttcgtaacttactgaatctggtcggcaaatgtgcgaaaatggaaggcttcatgtcaggtcagtatcaccaccgtatgggggaatttttcgaggaaatgactggctacatcaaacagggtaaaattaaatataaagaagatgtcaaagtcggcctggactcttttctggaagcctttaacagcatgttcactggcgaaaatattggcaaacccgtgatctatcttgggccacccctgcctaaataa

SEQ ID NO: 18 - DNA, 1050 bp, Rubus idaeus
atgggcgcgtcgggcggggagatgcaggtgagcaataaacaagtcatttttcgtgactacgttacgggcttcccgaaagaaagcgatatggagttaaccacacgttccatcacactgaaactcccgcagggcagcacaggtttgctgctgaaaaatctgtatctgtcgtgtgacccttacatgcgtgcgcggatgacgaatcaccatcggttgtcgtatgttgactcctttaaacctgggagtccgattattggatacggggtggcccgtgtgttagaatctggcaatcctaaattcaatccgggcgatctggtgtggggttttacgggttgggaagagtactcggttattactgcgacggaaagcctgtttaaaattcataacaccgatgtgccgctgtcgtactacactggattactgggcatgcccggcatgacagcctacgctggcttttacgaaatctgcagtcctaagaaaggcgagacggtgtatgtcagcgcggcatctggcgcagtggggcagctggttggtcaatttgcaaaattgaccggctgttacgtggttggcagcgcaggtagcaaagaaaaggttgacctgttaaaaaacaaatttggctttgatgaagccttcaattataaagaagaggcagatctcgacgctgctctgcgtcgctatttccccgatggcatcgatatctattttgaaaatgttggtggaaaaatgttggatgcggttttgccgaacatgcgtccgaaagggcgtattgcagtgtgtggcatgatttcacagtacaacctggaacagccggaaggcgttcgcaatctgatggctcttattgttaaacaggtacgcatggaaggctttatggttttcagctattaccacctgtatggtaaatttctggaaaccgtgttaccttatattaaacagggcaaaattacatacgtggaggatgtagtcgacgggttagataatgcgccggcggcgctgattgggctttactctggccgtaatgtcggaaaacaggtagtcgtcgtttcccgggagtga

SEQ ID NO: 19 - DNA, 990 bp, Rattus norvegicus
atggtacaagcgaaaacctggaccttgaaaaagcatttcgaagggtttcctacggacagcaattttgagctccgtacgactgaacttccgccgttaaataatggtgaagtcctccttgaagcactgtttctgagcgttgacccgtacatgcgtgtggcggcgaaaaaactgaaggaaggcgatagcatgatgggagaacaagttgcccgtgtcgtcgaaagtaaaaattccgccttcccgaccggtactattgtagtggccctcctgggatggacctcgcattctattagcgatggcaacggcttgcggaaattgccggcggagtggcctgataagttaccgttaagccttgcgcttggaactgtggggatgccggggctgaccgcgtacttcgggcttttagacatctgcggcttaaaaggcggcgagacggtcttggtaaatgctgcagccggcgcggttggtagcgttgtaggtcagattgcgaaactgaaaggttgtaaggtggtgggtacggcggggagtgatgaaaaagtcgcgtatctgaaaaaattagggtttgatgtcgcctttaattacaaaacggtgaagtccctcgaggaagcgttgcgcacggccagcccggatgggtatgattgctattttgataatgtcgggggcgaattctcgaatactgtcattctgcaaatgaagacctttggtcgtattgccatttgcggcgcgattagccaatataaccgtactggtccttgtccgccggggccgtcacccgaagtcattatctatcagcaactgcgcatggaagggtttattgtaacccgctggcagggggaagtgcgccagaaagcgttgactgaccttatgaactgggtttcagaaggtaagatccgctatcacgaatacatcactgaggggtttgaaaaaatgccggcggcgttcatgggtatgctgaaaggcgataatcttggtaaaactattgtgaaagcgtga

SEQ ID NO: 20 - DNA, 1038 bp, Raphanus sativus
atgggcacggccaccaataaacaggtgattttgcgtgattatgtgagcggttttccaaaagagagtgatttcgatatcacgaccacgacaacagtggagctgaaattgccgaagggctcaaacagtgtactggtgaagaatctgtatttaagctgcgatccatatatgcggtctcgcatgggcaaaccggcggcaagcggcattgcccaagcatacactcccggtaaaccgattcttggctttggtgttagtcgcgttatcgaatcgggccatccggattacaaagaaggcgacttgatgtgggggatcgtcggttgggaagaatatagtgtggttacgccaattccagacatgcactttaaaattcaccacacggatgtacctttgtcttattatacgggcctgttggggatgcccggtatgaccgcgtacgcgggtttttacgaggtctgcagcccgaagaaaggcgagactgtgtacgtttctgctgcatccggtgcggtaggccagttagttgggcagttcgccaagatgatgggttgttacgtggtgggtagcgccggcagcaaagagaaagtcgatctcctgaaaaataaatttgggttcgacgatgcgtttaactataaagaggaacaggacctcaccgcagctctgaaacgttgtttcccgcgcggcattgatatttatttcgaaaacgtagggggtaagatgctggatgcggttttactcaacatgaacgtgcacggccgtatcgcggtctgcggaatgatttctcagtacaacctcgagaatcatgaaggagttcacaatctcagcaatattatttacaagcgtattcgcatccaggggttcgcggttttcgatttttatgacaaatatgccaagtttttggagttcgttattccgtgcatcaaagaaggcaagattgcttatgtggaggatgtagcggaaggtctggaaaaggcgccggaagcgctgatcgggctgtttcatgggaacaatgtcgggaagcaggttgtggttgtagctcacgaataa

SEQ ID NO: 21 - DNA, 1041 bp, Tarenaya hassleriana
atgggcacagctacctctaaacaggtgattctccgcgattatgtatctggattccccaaagaaagcgatctcgaaatcaccgccaccgccgtggaactgaaagtgccggccaatagtagtagcgtgcttgtaaagaatttatatctctcatgtgatccgtacatgcgtatgcggatgggcaaaccgaatcctgcgaccgcagccttagcgcaggcgtataacccgggcaagccggtttttgggttcggagttagcaaggttattgatagcggtcatcccgatttccagaagggcgatttgctgtggggcattgttggttgggaagaatatagcgtcatcacccccacgccgaacctccatttcaaaatccatcacacggacgtcccactgtcttattacacgggccttctgggtatgcccggcatgacggcctatgcaggcttttacgagatctgcaccccgaagaaaggtgaatatgttttcgtaagcgccgcatccggtgcggttgggcagctggtggggcaatttgcgaaaatgatggggtgttacgtcgtggggtcggctggcagtaaagaaaaagtggatctcctgaaaaacaaatttggcttcgatgatgcatttaactacaaagaagaaccggatctggccgccgctctgaaacgctgttttccggaaggcattgatatctattttgagaacgtaggcggtaagatgctcgatgccgtccttgttaatatgcgtatgaacggccgtatcgcggtctgtgggatgatcagccaatacaatctggaaaacccggagggcgtgcataatctgaccaccattatctacaatcgcattcgtatcgaaggctttgttgtgttcgattatttcgaccggcattcgaaatttctggatttcgtcctgccttacatccgtgaaggaaagattacttatgtggaagatgttgcggaaggcctggaaagtgggccaagtgcactcattggcctgtttcatggcaaaaatgttggcaaacagttagtcgcggtcgcgccggaataa

SEQ ID NO: 22 - DNA, 1041 bp, Tarenaya hassleriana
atgggcaccgcgagcaataagcaagtcatcctgcgggactacgtcaccggattccctaaaggttccgatctcattgttaccacgacagctgttgaactgcgtgtacaaaccgagtctcctactgttctggtgaaaaacttgtatctgtcgtgtgacccctatatgcgtattcgtatgggcaaacctgacccaagcagtccagcactgtcccagccctatgaaccgggtaaacccctcctcggttttggggtaagccgcgtgatcgattctacccatcctgacttcgagaaaggagacttactgtggggatttgtgggttgggaagaatatagcgtcattaccccgattcctaacgtgcataacaaaattcaccacatggatgttccgctcagctattatactggcctgttgggcatgccgggtatgacagcatatgttggcttttatgacatttgcgccccgcgcaagggcgaatttgtgtttgtttcatcggcgtctggtgcggtgggacagctcgtaggtcaattcgctaaactgatgggctgtcatgtcgtgggttctgcgggctcaaaagaaaaggtcgatttgttgaaaaataaatttggctttgatgacgcctttaactataaggaagagccggatctggacgccgcgctgaagcgctgctttcctgatggcattgacatttattttgaaaacgttggtgggaaaatgctggatgccgttttgctgaacatgcggatgaatggccgcatcgctgtctgcgggatgattagtcaatataacctggaagatccagagggcgtccataatctggcgacggttatttacaaacgcatccgcatccagggatttattgtaagcgattatttccagcgtcacagcgaatttcttgattttgtgctgccgtatatccgcgaacgcaagatcacatacgtggaagacactgtgcaaggtttagaaaacggtccatctgctcttattggtctgttccatggaaacaatgtgggcaagcaggtcgtggttgtggcaccggaatga

SEQ ID NO: 23 - DNA, 1056 bp, Zingiber officinale
atgggcgccagcgccgaagacgtagtggtagtgaataaacaggtacttcttaaacattttatccctgaaggtgccccgaaggaaacagacatggaactggtcaccactggcacgattcgtctgcgcgttccggaagggtcaaacgcggtactgctgaaaaatttatatctgtcttgtgacccgtacatgcgtatgcgtatgacgaagcacgaggaagcctcgtatgtggacgacttcgtgccgggggcccctatcaccgggtttggggtgggcaaagtggtggatagcagtcatccggatttcaaaacgggtgactatgtctgggggctcatcggctgggaagagtacagcctcattaccaagccacaaggcctgtttaaaattcatcacactgaaatcccgctgagctattacaccggcatccttggcatggtggggttaacggcgtatgtcggtttttacgacatttgttcgccgaaaaaaggcgaacgtgtcttcgtgtctgcggccgcaggcgcggtcggccaaattgtcggccagtttgcaaagcagtttggttgttatgtcgttggttccgcaggcagcgatgaaaaagtcaacctgctcaaaaccaaattcggttttgatgaagcgtttaattataaaaaagaaccggatcttacaaaagcgctgaaacgctatttccccgagggtatcgatatttattttgaaaatgttggtgggccgatgctcgaggcagtgctgcacaatatgcgcattaaagggcgcatcgcagcgtgcggaatgatcagccagtataacctggaaaaaccagaaggcgtccataatctctttctgattgtcggcaagcgcatccgcctggaaggcttccttgtctttgatcactatgggagctaccctgaatttgaagaaaaagtagttcaactgattaaagaagagaaaattaaatatttagaagatatcgtggagggcctggaaaacgccccggcggcgcttatcggcctgtttgagggtcgtaacgtgggcaaacaagtcgtggttgtaagccgtgagtaa

SEQ ID NO: 24 - protein, 346 aa, Arabidopsis alpina
MGTATNKQVIFKDYVSGFPKESDFDFTTTTVELRVPEGSNSVLVKNLYLSCDPYMRIRMGKPDPETAALAAAYPVGQPIIGYGVARVIESAHPDYKKGDLLWGFVGWEEYSLFTPVHNLHFKIHHTDVPLSYYTGLLGMPGMTAYAGFYEICSPKKGETVYVSAASGAVGQLVGQFAKMMGCYVVGSAGSKEKVDLLKTKFGFDDAFNYKEEQDLTAALKRCFPKGIDIYFENVGGKMLDAVLLNMNPHGRIAVCGMISQYNLQDQEGVHNLSSIIYKRIRIQGFVVSDFYDKYSKFLEFVVPCIKEGKITYVEDVAEGLEKGPEALVGLFHGKNVGKQVVVVARE

SEQ ID NO: 25 - protein, 345 aa, Arabidopsis thaliana
MTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFVVSDFYDKYSKFLEFVLPHIREGKITYVEDVADGLEKAPEALVGLFHGKNVGKQVVVVARE

SEQ ID NO: 26 - protein, 346 aa, Brassica cretica
MGASNNKQVILRDYVAGFPKESDLVFNDATVDLSVPAGSNKVLVKNLFLSCDPYMRIRMSKPDPSTAALALPYKPGQPIYGYGVSKVIESGHPDYTKGDLLWGIVGWEEYSVITLTPYSHFKILHTDVPLSYYTGLLGMAGMTAYSGFYEICSPKKGETVFVSAASGAVGQLVGQLAKIMGCYVVGSAGSNEKVELLKNKFGFDEAFNYKEEQDLNAALKRCFPEGIDIYFENVGGKMLDAVLLNMKLNGRIAVCGMISQYNLEEQEGVRNLATVIYKRVRLQGFVVSDYYHKYSNFLEFVLPYIREGKITYVEDVAEGLENGPSALIGLFHGKNVGKQLIVVARE

SEQ ID NO: 27 - protein, 333 aa, Brassica rapa
MGATNKQVILRDYVSGFPKESDFEITTTTVELRLPEGSNSVLVKNLYLSCDPYMRSRMGKPSAFALAQAFTPGKPILGFGVSRVIESGHPGYKQGDLLWGIVGWEEYSVVTPEMHFKIHDTDVPLSYYTGLLGMPGMTAYAGFHEVCSPKKGETVYVSAASGAVGQLVGQFAKMMGCYVVGSAGSKEKVDLLKNKFGFDDAFNYKEEQDLSAALKRCFPKGIDIYFENVGGKMLDAVLLNMNMHGRIAVCGMISQYNLEDQEGVHNLTSIIYKRIRIQGFVVFDYFEKYSNFMEFVIPCIKEEKIAYVEDVAEGLEKGPEALVGLFYGKNVGK

SEQ ID NO: 28 - protein, 329 aa, Cavia porcellus
MVKAKSWTLKKHFQGKPTQSDFELKTVELPPLKNGEVLLEALFLSVDPYMRIASKRLKEGAVMMGQQVARVVESKNSAFPAGSIVLAQSGWTTHFISDGKGLEKLLTEWPDKLPLSLALGTIGMPGLTAYFGLLEVCGVKGGETVLVSAAAGAVGSVVGQIAKLKGCKVVGAAGSDEKIAYLKQIGFDAAFNYKTVNSLEEALKKASPDGYDCYFDNVGGEFLNTVLSQMKDFGKIAICGAISVYNRMDQLPPGPSPESIIYKQLRIEGFIVYRWQGDVREKALRDLMKWVLEGKIQYHEHVTKGFENMPAAFIEMLNGANLGKAVVTA

SEQ ID NO: 29 - protein, 345 aa, Capsella rubella
MGTATNKQVTLKDYVSGFPKESDFDFTTTTVELRVPEGSNSVLVKNLYLSCDPYMRTRMSKPKPSTALAQAYALGQPIFGFGVSRVIESGHPDYKKGDLLWGIVGWEEYSVITPTPDVHFKIQHTDVPLSYYTGLLGMPGMTAYAGFYEICSPKEGETVYVSAASGAVGQLVGQFAKMMGCYVVGSAGSKEKVDLLKTKFGFDDAFNYKEEPDLSAALKRCFPKGIDIYFENVGGKMLDAVLLNMNVHGRIAVCGMISQYNLENQEGVHNLSNIIYKRIRIQGFAVFDFYDKYSKFLEFVLPHIKEGKITYVEDVADGLEKGPEALVGLFHGKNVGKQVVVVARE

SEQ ID NO: 30 - protein, 345 aa, Capsella rubella
MGTATNKQVTLKDYVSGFPKESDFDFTTTTVELRVPEGSNSVLVKNLYLSCDPYMRTRMSKPKPSTALAQAYALGQPINGYGVSRVIESGHPDYKKGDLLWGFVGWEEYSVITPMNRMHFKIQHTDVPLSYYTGLLGMPGMTAYAGFYEICSPKEGETVYVSAASGAVGQLVGQFAKMMGCYVVGSAGSKEKVDLLKTKFGFDDAFNYKEEQDLSAALKRCFPKGIDIYFENVGGKMLDAVLLNMNPHGRIAVCGMISQYNLENQEGVHNLSNIIYKRIRIQGFVVSDFYDKIPKFLEFVLPQIKEGKITYVEDVAEGIEKGPEALIGLFHGKNVGKQVVVIARE

SEQ ID NO: 31 - protein, 346 aa, Camelina sativa
MGTATNNKQVTLRDYVSGFPKESDFDFTTSTVELRVPEGSNSVLVKNLYLSCDPYMRTRMGEPKPSTALAQAYTLGKPIYGYGVSRVIESGHPDYKKGDLLWGIVGWEEYSVITPAPHMHFKIQHTDVPLSYYTGLLGMPGMTAYAGFYEICSPKEGETVYVSAASGAVGQLVGQFAKMMGCYVVGSAGSKDKVDLLKTKFGFDDAFNYKEEPDLSAALKRCFPKGIDIYFENVGGKMLDAVLLNMNVHGRIAVCGMISQYNLENQEGVHNLSNIIYKRIRIQGFAVFDFYDKYSKFLEFVLPHIKEGKITYIEDVADGLEKGPEALVGLFHGKNVGKQVVVVARE

SEQ ID NO: 32 - protein, 344 aa, Eutrema salsugineum
MGTATNKQVILRDYVTGFPKESDFDFTTTTVELRVPEGSNSVLVKNLYLSCDPYMRSRMGKPADSALAQAFTVGQPILGYGVSRVIESGHPDYKKGDLLWGIVGWEEYSVITPIPDMHFKIHHTDVPLSYYTGLLGMPGMTAYAGFYEICSPKKGETVYVSAASGAVGQLVGQFAKMMGCYVVGSAGSKEKVDLLKTKFGFDDAFNYKEEQDLSAALKRCFPKGIDIYFENVGGKMLDAVLLNMNMHGRIAVCGMISQYNLEDQEGVHNLSTIIYKRIRIQGFVVYDHYDKYSKFLEFVLPCIKEGKIAYVEDVAEGLEKGPEALVGLFHGKNVGKQVVVVARE

SEQ ID NO: 33 - protein, 266 aa, Eutrema salsugineum
GLGVSKVIDSGHPDYKKGDLLWGLVGWEEYTVITLTPYSHFKIHHTDVPLSYYTGLLGMPGMTAYAGFYEICSPKKGETVFVSAASGAVGQLVGQFAKLMGCYVVGSVGSKEKVDLLKTKFGFDDAFNYKEEQDLNAALKRCFPEGIDIYFENVGGKMLDAVLINMKLHGRIAVCGMISQYNLEDPEGVHNLATILYKRIKLQGFGVFDFYDKYYNFLDFVLPYIREGKITYIEDIAEGLESGPAALLGLFHGKNFGKQLFVVARE

SEQ ID NO: 34 - protein, 349 aa, Homo sapiens
MHHHHHHSSGVDLGTENLYFQSMTKTWTLKKHFVGYPTNSDFELKTSELPPLKNGEVLLEALFLTVDPYMRVAAKRLKEGDTMMGQQVAKVVESKNVALPKGTIVLASPGWTTHSISDGKDLEKLLTEWPDTIPLSLALGTVGMPGLTAYFGLLEICGVKGGETVMVNAAAGAVGSVVGQIAKLKGCKVVGAVGSDEKVAYLQKLGFDVVFNYKTVESLEETLKKASPDGYDCYFDNVGGEFSNTVIGQMKKFGRIAICGAISTYNRTGPLPPGPPPEIVIYQELRMEAFVVYRWQGDARQKALKDLLKWVLEGKIQYKEYIIEGFENMPAAFMGMLKGDNLGKTIVKA

SEQ ID NO: 35 - protein, 344 aa, Microthlaspi erraticum
MGTATNKQIILRDYVSGFPKESDFEFTTTTVELRAPEGSKSLLVKNLYLSCDPYMRSRMGKPAASGIAQAFTVGKPIIGFGVSRVIESGHPDYKKGDLIWGIVGWEEYSVVTPIPDMHFKIHDTDVPLSYYTGLLGMPGMTAYAGFYEICSPKKGETVYVSAASGAVGQLVGQFAKMMGCYVVGSAGSQEKVDLLKTKFGFDDAFNYKEEQDLSAALKRCFPKGIDIYFENVGGKMLDAVLLNMNTFGRVAVCGMISQYNLEDHEGVYNLTSIIYKRIRIQGFAVFDHYDKYSKFLELVIPSIKEGKIAYVEDVAEGLEKAPEALVGLFHGKNVGKQVVVVARE

SEQ ID NO: 36 - protein, 346 aa, Microthlaspi erraticum
MGTTTNKQIIFPEYVSGFPKESDLNITTTTIDLKLPEGSTSVLVKNLYLSCDPYMRICMGKPDPLANSLVPPYITGEPIVGLGVSKVIDSGHPDYKKGDLLWGLVGWEEYTVITLTPYSHFKINHTDVPLSYYTGLLGMPGMTAYAGFYEVCSPKKGETVFVSAASGAVGQLVGQFAKMMGCYVVGSAGSKEKVDLLKEKFGFDEAFNYKEEQDLSSSLKRCFPEGIDIYFENVGGKMLDAVLVNMKSQGRIAVCGMISQYNLVDTEGVHNLPTILYKRIKIQGFRVFDFYDKYSKFLDFVLPHIREGNITYIEDITEGLESGPSALLGLFHGKNVGKQLFVVARE

SEQ ID NO: 37 - protein, 343 aa, Nicotiana tabacum
MAEEVSNKQVILKNYVTGYPKESDMEIKNVTIKLKVPEGSNDVVVKNLYLSCDPYMRSRMRKIEGSYVESFAPGSPITGYGVAKVLESGDPKFQKGDLVWGMTGWEEYSIITPTQTLFKIHDKDVPLSYYTGILGMPGMTAYAGFHEVCSPKKGETVFVSAASGAVGQLVGQFAKMLGCYVVGSAGSKEKVDLLKSKFGFDEAFNYKEEQDLSAALKRYFPDGIDIYFENVGGKMLDAVLVNMKLYGRIAVCGMISQYNLEQTEGVHNLFCLITKRIRMEGFLVFDYYHLYPKYLEMVIPQIKAGKVVYVEDVAHGLESAPTALVGLFSGRNIGKQVVMVSRE

SEQ ID NO: 38 - protein, 345 aa, Olimarabidopsis pumila
MGTATNKQVILKDYVSGFPKESDFDFTTTTVELKVPEGSNSVLVKNLYLSCDPYMRTRMGKPKPSTALAQAYALGQPIYGYGVSRVIESGHPDYKKGDLLWGIVGWEEYSVITPTPDVHFKIQHTHVPLSYYTGLLGMPGMTAYAGFYEICSPKEGETVYVSAASGAVGQLVGQFAKMMGCYVVGSAGSKEKVDLLKTKFGFDDAFNYKEEPDLSAALKRCFPKGVDIYFENVGGKMLDAVLLNMNVHGRIAVCGMISQYNLENQEGVHNLSNIIYKRIRIQGFAVFDFYDKYSKFLEFVLPQIKEGKIKYVEDVADGLEKGPEALVGLFHGKNVGKQVVVIARE

SEQ ID NO: 39 - protein, 341 aa, Plagiochasma appendiculatum
MAGTEVTNRQILFKNFVVGWPQESDMEFVTTKKKLELREGQKDVIVRVLYLSCDPYQRGRMRNSTDSYIPPYTPGKPIEGYGLGKVILSSDPAFNEGDIVSGLMGWEDYSYGFRLVKIDDLALPISYYLGALGMAGFTAYVGFFRICEPKKGDTIYVSAASGAVGQMVGQFAKLFGCRVVGSAGSQQKVDLLTSKFGYDEAFNYKEEPDLDAALKRCCPDGIDIYFENVGGKMLDAVLVNMKVFGRIAVCGMVSQYNKEVQDPIYNLNYTVKRRLKIHGFLQSDHMDVQPEFFKNVIQWFKEGKLVYVEDIADGLEKGPAALIGLLAGKNVGKQSVKIADP

SEQ ID NO: 40 - protein, 351 aa, Pinus taeda
MEQRVPNRELILVAYANEGPVTDSHLNIRETKLDLGSVGKDGSSGDVAVQNLWISVDPYLRQLMKESDDGLYLPSFPLNQAIRSILVGKVVASANPAFEVGDIVSGFYQVSEYAIVPRGDLMKIDTSVVKPSDYLGLLGMPALTAWAGFIIVGEPKPGDEVFVSAAAGSVGMLVGQLAKIKSCRVVGSAGSDQKVKLLKEFGFDDAFNYKCETDLDAALSRYFPRGIDIYFDNVGGRMLEAVLNHINMKARIPLCGMISQYNQEWKQRFGVRNLLNLVGKCAKMEGFMSGQYHHRMGEFFEEMTGYIKQGKIKYKEDVKVGLDSFLEAFNSMFTGENIGKPVIYLGPPLPK

SEQ ID NO: 41 - protein, 349 aa, Rubus idaeus
MGASGGEMQVSNKQVIFRDYVTGFPKESDMELTTRSITLKLPQGSTGLLLKNLYLSCDPYMRARMTNHHRLSYVDSFKPGSPIIGYGVARVLESGNPKFNPGDLVWGFTGWEEYSVITATESLFKIHNTDVPLSYYTGLLGMPGMTAYAGFYEICSPKKGETVYVSAASGAVGQLVGQFAKLTGCYVVGSAGSKEKVDLLKNKFGFDEAFNYKEEADLDAALRRYFPDGIDIYFENVGGKMLDAVLPNMRPKGRIAVCGMISQYNLEQPEGVRNLMALIVKQVRMEGFMVFSYYHLYGKFLETVLPYIKQGKITYVEDVVDGLDNAPAALIGLYSGRNVGKQVVVVSRE

SEQ ID NO: 42 - protein, 329 aa, Rattus norvegicus
MVQAKTWTLKKHFEGFPTDSNFELRTTELPPLNNGEVLLEALFLSVDPYMRVAAKKLKEGDSMMGEQVARVVESKNSAFPTGTIVVALLGWTSHSISDGNGLRKLPAEWPDKLPLSLALGTVGMPGLTAYFGLLDICGLKGGETVLVNAAAGAVGSVVGQIAKLKGCKVVGTAGSDEKVAYLKKLGFDVAFNYKTVKSLEEALRTASPDGYDCYFDNVGGEFSNTVILQMKTFGRIAICGAISQYNRTGPCPPGPSPEVIIYQQLRMEGFIVTRWQGEVRQKALTDLMNWVSEGKIRYHEYITEGFEKMPAAFMGMLKGDNLGKTIVKA

SEQ ID NO: 43 - protein, 345 aa, Raphanus sativus
MGTATNKQVILRDYVSGFPKESDFDITTTTTVELKLPKGSNSVLVKNLYLSCDPYMRSRMGKPAASGIAQAYTPGKPILGFGVSRVIESGHPDYKEGDLMWGIVGWEEYSVVTPIPDMHFKIHHTDVPLSYYTGLLGMPGMTAYAGFYEVCSPKKGETVYVSAASGAVGQLVGQFAKMMGCYVVGSAGSKEKVDLLKNKFGFDDAFNYKEEQDLTAALKRCFPRGIDIYFENVGGKMLDAVLLNMNVHGRIAVCGMISQYNLENHEGVHNLSNIIYKRIRIQGFAVFDFYDKYAKFLEFVIPCIKEGKIAYVEDVAEGLEKAPEALIGLFHGNNVGKQVVVVAHE

SEQ ID NO: 44 - protein, 346 aa, Tarenaya hassleriana
MGTATSKQVILRDYVSGFPKESDLEITATAVELKVPANSSSVLVKNLYLSCDPYMRMRMGKPNPATAALAQAYNPGKPVFGFGVSKVIDSGHPDFQKGDLLWGIVGWEEYSVITPTPNLHFKIHHTDVPLSYYTGLLGMPGMTAYAGFYEICTPKKGEYVFVSAASGAVGQLVGQFAKMMGCYVVGSAGSKEKVDLLKNKFGFDDAFNYKEEPDLAAALKRCFPEGIDIYFENVGGKMLDAVLVNMRMNGRIAVCGMISQYNLENPEGVHNLTTIIYNRIRIEGFVVFDYFDRHSKFLDFVLPYIREGKITYVEDVAEGLESGPSALIGLFHGKNVGKQLVAVAPE

SEQ ID NO: 45 - protein, 346 aa, Tarenaya hassleriana
MGTASNKQVILRDYVTGFPKGSDLIVTTTAVELRVQTESPTVLVKNLYLSCDPYMRIRMGKPDPSSPALSQPYEPGKPLLGFGVSRVIDSTHPDFEKGDLLWGFVGWEEYSVITPIPNVHNKIHHMDVPLSYYTGLLGMPGMTAYVGFYDICAPRKGEFVFVSSASGAVGQLVGQFAKLMGCHVVGSAGSKEKVDLLKNKFGFDDAFNYKEEPDLDAALKRCFPDGIDIYFENVGGKMLDAVLLNMRMNGRIAVCGMISQYNLEDPEGVHNLATVIYKRIRIQGFIVSDYFQRHSEFLDFVLPYIRERKITYVEDTVQGLENGPSALIGLFHGNNVGKQVVVVAPE

SEQ ID NO: 46 - protein, 351 aa, Zingiber officinale
MGASAEDVVVVNKQVLLKHFIPEGAPKETDMELVTTGTIRLRVPEGSNAVLLKNLYLSCDPYMRMRMTKHEEASYVDDFVPGAPITGFGVGKVVDSSHPDFKTGDYVWGLIGWEEYSLITKPQGLFKIHHTEIPLSYYTGILGMVGLTAYVGFYDICSPKKGERVFVSAAAGAVGQIVGQFAKQFGCYVVGSAGSDEKVNLLKTKFGFDEAFNYKKEPDLTKALKRYFPEGIDIYFENVGGPMLEAVLHNMRIKGRIAACGMISQYNLEKPEGVHNLFLIVGKRIRLEGFLVFDHYGSYPEFEEKVVQLIKEEKIKYLEDIVEGLENAPAALIGLFEGRNVGKQVVVVSRE

SEQ ID NO: 47 - DNA, 861 bp, Acetoanaerobium noterae
atggcagattttgaattcaacccgatgcgtagcctgatctatgtggatgttgtgaaagaagattatcgccacatgctgaaacattggctgtatcgccatcatattccggatagcattagtcagtttggtccgtatgttaccaaatatgcattttatccggccctgccgaccccgccggaaggtgacagatttggcaccattcgtatgcagctgaccgaacattattggaccgttaatccgtttaccagcaattttaaaaacaaggcctttaccgaattctttccgattgatattctgaaatggcagggtaatattccggatgatagcaaagttgcagcactggaaggtgaagaagttgttaaactggaaggtgacgatgcacgcgccagtggtggtgacaatggttgtccgccgtttatttttgcatttgtgccgatttgttgggaagaagattttaaaggtagccagcgtaccgttgaagatggtccgaattatcgttggcagtttgtgatgaaatatccggaaggtgtgaccatgaaagaaggcgataaatggttttatgaagaaatgattccgaagtttaaggatatgccggatgttctgcgtattctgagtagtaaaattattcagcaggtgaatggctgcccgtatcatcgtgttgtggaaatgtggtttaattgcccgagcgcatggtatcgtagtgccgttgaaaaagcaaaagaaattaagaaaccgagttgggcaaaatatgatgtgtttccgtatctgaaaccgaaatttgaaattgcaagtctgtttctgaccgatattgcagaaagcgataattatagccagtatcgtggttatattaccatgcgctaa

SEQ ID NO: 48 - protein, 286 aa, Acetoanaerobium noterae
MADFEFNPMRSLIYVDVVKEDYRHMLKHWLYRHHIPDSISQFGPYVTKYAFYPALPTPPEGDRFGTIRMQLTEHYWTVNPFTSNFKNKAFTEFFPIDILKWQGNIPDDSKVAALEGEEVVKLEGDDARASGGDNGCPPFIFAFVPICWEEDFKGSQRTVEDGPNYRWQFVMKYPEGVTMKEGDKWFYEEMIPKFKDMPDVLRILSSKIIQQVNGCPYHRVVEMWFNCPSAWYRSAVEKAKEIKKPSWAKYDVFPYLKPKFEIASLFLTDIAESDNYSQYRGYITMR

SEQ ID NO: 49 - DNA, 20 bp, Artificial Sequence
  Forward primer
aagcttgcggccgcataatg

SEQ ID NO: 50 - DNA, 30 bp, Artificial Sequence
  Reverse Primer
ggtatatctccttattaaagttaaacaaaa

SEQ ID NO: 51 - DNA, 49 bp, Artificial Sequence
  Forward Primer
tttaactttaataaggagatataccatggcagattttgaattcaacccg

SEQ ID NO: 52 - DNA, 49 bp, Artificial Sequence
  Reverse Primer
ttaagcattatgcggccgcaagcttttagcgcatggtaatataaccacg

SEQ ID NO: 53 - DNA, 831 bp, Fusibacter sp. 3D3
agctatgtgccggaattcaaaccgatgcgtagctttattctgaataatgtgcgcagcgataatgatcgcccgaaagcatttcgttggctgtataaacagcatgttccggaaagcattagtcagtttcgtccgtattgtaccaaatatgcaacctatcatgccctgccgctggttgaaggtgccgaacagtttggcacctataattggattatgaccgaacattattggaattttaacatcttccaggataagggcctgggccatagtcgtggcctggcattcactgaagcatatccgcatgattttatggaaattacctgccagccgccgagtaataccctgcgccagagcggctggcagggtagtgccgaaggttatcatccgattgtgttttgttttgcaccgattttctgggaaaatgattttaaaggcagcgaacgtctgaccgatgatggtgccaattttcgttggttatttttcctgagttacccggaaggtgttaccgtggaagaaggcgatcagtggtttctggaaacctttgccaaagaagcagccgaaatgccggaagttattcgttttctgagtagtcgcgttatggatagtccgcgcattaatccgtttgatcgtattgttgaactgtggtttgatgatagcgaagcctggcataatgccatggtgaaaaatggccatctgtttaccaaaccggcatggagcaaagtggatcagtttccgtttttcgaagcctataaagatattaccggcgtttttattatggatcgcccggatagcgatcatctggcccagtggcagggttatattaccacccgctaa

SEQ ID NO: 54 - DNA, 837 bp, Tepidanaerobacter acetatoxydans
gataccgaattcaaaccgatgcgtagtctgatctatgtgggcgttgccaaagaagaatatcgtcataaactgaaaaactggctgtatcgttatcatattccggatagtattagccagtttgcaccgtatgttaccaaatatgcattttataacgcgctgccggtgccgccggaaggtgaacgttttggtgcatataatatgcagctgaccgaacattattggctgattaatccgatgaatccgcagctgaaaattaaggcattcactgaatttttcccggtggatgttctgaaatggcagggtaatattccggatgatgcagatgcagatagcgttaatattgaaggcgatgccgcacgcagtagtggtggtgacaatggcatgccgccgtttatttttgcattcattccgttttggtgggaagaagattttaaaggtgcaggtcgtaccgttgaagatggcccgaattatcgctggcagtttctgattcagtatccgaatggcattagtctggaagaaggtgaccagtggttttataaaaaactggttccggcctttaaagaaatgccggaagtgaatcgttttctgaccagccgcgttattcgcgatgtgaatgattgtcagtttcatcgcgttattgaaatgtggtttgatggtccggaagaatggtatcgcgccgccgtggaaaaaggcaaaagcattgaacgtccgcattgggcaaccgccgaagaatttccgtatctgaaaccgaaatttcagattgcaagcctgtttctgaccgatattgcagatagtgataattatagccagtatcgcggttatattaccatgcgttaa

SEQ ID NO: 55 - DNA, 857 bp, Clostridium sp. JN500901
agcattgaattcaagccgatgcgtagcctgatctatgtggatgtggttaaagaagattatcgtcacatgctgaaacattggctgtattatcatcatattccggatagcattagtcagtttggcccgtatgttaccaaatatgccttttatccggccctgccgaccccgccggatggtgagagatttggtacccatcgtatgcagctgaccgaacattattggctggttaatccgctgaccccggaattcaaaaataagaccatgaccgaatatttcccggttgatgtgctgaaatggcagggtaatgttccggatgataatgaagttgcaattctggaaggtaaagaaaccgaaaaagtggaaagcggtgacgcagcccgtagtaccggcggtgacaatggtatgccgccgtttatttgggcctttgtgccgattagttgggaagaagatctgaaaggtgaaggtcgtagtatggaagatggtccgaattatcgttggcagtttgttatgaaatacccggatggtgtgccgatggaagaaggcgataaatggttttataaagaagtgattccgaagtttcaggatagcccggatgttacccgtattctgaccagcaaaattattcagaatatcaatgactgcccgtatcagcgtattgtggaaatgtggtttaattgtccgagtggctggcatcgcgccgcagttgaaaaagcacgtgaaattaagaaaccgagttgggcaaaatatgatgaatttccgtatctgaaaccgaaatttgaaattagtagcctgtttctgaccgatattgccgaaagtgataattatacccagtatcgtggttttattccgatgcgtta

SEQ ID NO: 56 - DNA, 858 bp, Acetoanaerobium noterae
gcagattttgaattcaacccgatgcgtagcctgatctatgtggatgttgtgaaagaagattatcgccacatgctgaaacattggctgtatcgccatcatattccggatagcattagtcagtttggtccgtatgttaccaaatatgcattttatccggccctgccgaccccgccggaaggtgacagatttggcaccattcgtatgcagctgaccgaacattattggaccgttaatccgtttaccagcaattttaaaaacaaggcctttaccgaattctttccgattgatattctgaaatggcagggtaatattccggatgatagcaaagttgcagcactggaaggtgaagaagttgttaaactggaaggtgacgatgcacgcgccagtggtggtgacaatggttgtccgccgtttatttttgcatttgtgccgatttgttgggaagaagattttaaaggtagccagcgtaccgttgaagatggtccgaattatcgttggcagtttgtgatgaaatatccggaaggtgtgaccatgaaagaaggcgataaatggttttatgaagaaatgattccgaagtttaaggatatgccggatgttctgcgtattctgagtagtaaaattattcagcaggtgaatggctgcccgtatcatcgtgttgtggaaatgtggtttaattgcccgagcgcatggtatcgtagtgccgttgaaaaagcaaaagaaattaagaaaccgagttgggcaaaatatgatgtgtttccgtatctgaaaccgaaatttgaaattgcaagtctgtttctgaccgatattgcagaaagcgataattatagccagtatcgtggttatattaccatgcgctaa

SEQ ID NO: 57 - DNA, 831 bp, Parasporobacterium paucivorans
ggtccggaatttcagccgatgcgcagcctgatctatgtggattgtgttaaagaagaataccgccatcgtctgcagaattggctgtatcattatcatgttccggatagtattagtcagtttgcaccgtatgtgagcaaatattgtttttataacgccctgccgaccccgccggaaggtgaacgttttggtaccattcgtcagcagctgaccgaacattattggatggtgaatccgtttaccgaacattttaaaaataaggccctgaccgaatattttccgtgcgatgttctgaaatggcagggcaatattccggatgatgatctggaagttaattttgaaggtgacgaagcccgcagtaaaggcggtcataatggctgtgcaccgtttatttttgcctttctgccgctgtgttgggaagaagattttaaaggcaaagaacgtaccattgaaaatggtccgaattatcgttggaattttgtgatgaaatacccggaaggtaccagcatggaagaaggtgacaaatggttttatgaaaaaatgatcccggcctttcaggaaatgccggaagttacccgcattctgaccagcaaagttattcagagcattaatggttgcgcctttcatcgtgttgtggaaatgtggtttgatggcccgaatgcctggtatcaggccgccgtggtgaaagcaaaaaatattgaaaaaccgagttgggcaagcgttgatgcctttccgtatctgcaggcaacctttgaatttgatggtatttttctgaccgattatgccgccagtgataatatgagccagtatcgtggttatctggccatgcgttaa

SEQ ID NO: 58 - DNA, 834 bp, Lachnoclostridium sp.
ggtaccgaatttgatccgatgcgcagttttatctatgttgatgtgattaaggaggaatatcgccataaactgcagcattggctgtattttcatcatattccggaaagtattagccagtttgaaccgtattgtaccaaatatgcattttataacgccctgccggttccggaaggtggtgaacgttttggcaccgcccgcatgcagattaccgaacatagctggctggtgaatattatggaccctgcactgaaagttaaagcatttcaggaatatatgccgattgaagcactgaaatggcagggcaccgttccggaaaccgccggcgatgtgctgatgagcggcgatgatgcacgctgcaccggtggcaataatggttgcatgccgtttgtgtttgccttttgcccgatgtggtgggaagaagatgtgaaaggtgccggtcgtaccctgaccgaaggcgccaattatcgttggaattttgcactgacctatccggaaggtattagtaaagaagaaggtgacaaatggctgtttgaagaagtttttccgtattttgaagcaaataactgctgtacccgtattctgaccagtaaagtgcgccaggaaattaatggttgtccgatgtatcgcgtggttgaaatgtggtttaccggcccgaccgcctggcatcatctggccgtggaaaccgcagataaaattaagagcccgagctgggccaaagatggtgacgtgtttccgtatctgaaaagtggctttgaaattcgcggcgcatttgtgaccgattatccgacctgtgatgcctatagtcagtatcgcggttatattccgcgtcgctaa

SEQ ID NO: 59 - DNA, 870 bp, Clostridium sp. SY8519
gcagattttaagttcgaaccgatgcgtagctttatctatgttaattgtgcaaaagaggactatcgtccgaaactgcagcgctggctgtataaaacccatgttccggatagcattggtcagtttgaaccgtatgttaccaaatatgcattttatccgagttttccgaccccgccggaaggtgaacgttttggttattgcagtatgcagctgaccgaacatcattggatggtgagtgattttgatgaacgtctgggcattaaggcaattgccgaaacctttccgccggaagttctgctgtggcagggcaatattaccgaaggcatgctggaagcaatgaaaaagaatggtggtgcaccgggcgatgatggcggtgacagtggtaatgaagcccgcagtaccaatatggaaggcgcaaatccgtttattttctgttttgcaccgtattggtgggaagatgatattaagggcaaaggtcgtaccattgaagatggtgcaaattatcgttggaatgtggccattggttatccggaaggtgtgagtgccgaagaaggcgatcgctggctgtttgaagaagtgattccggtttttgaagccgcaccggaatgcacccgtattctgcatagtaaagttaaaaaagagatcaacggtggtagcggcatggaacatattctggaattttggtttgaaaaccagagtggttggtatcagaccgccgttgaagccaccaaaaccctgaaaaaaccggcctgggcacagcaggatcagtttccgtttctgaaaccgtatcataaattcattagcgtggccgtggcagattttaccatggccaataatctggttaattatcatggctatatcccgcgccgttaa

SEQ ID NO: 60 - DNA, 804 bp, Roseburia sp.
cgcagtctgatctatgttgatgtggccaaagaacagtatcgcgtgaaactgaatcattggctgtatgcagtgcatgttccggaaagcattagtaaatttcgcccgtatgttagccagtatgcattttataatgcactgccggtgccgccgctgggtgaacagtttggtaccgttcgtatgcagctgaccgaacattattggcaggtgaatccgatggatgtgagcatggccgttaatgtgtttaccgaaacctttccgaccgaagtgctgatttggcagggcaatatgccggaaaatgccattgccctgaattatgaaggtgacgaagcacgcagcgcaaaagttgaaaattgtaatccgtttgttttcgcctttctgccgattagttgggaagaagatctgaaaggtgcaggccgcaccatggcagatggtccgaattatcgctggaattttgcactgcattatccggatggtgttagtgttgaaaccggcgatcagtggttttatgaaaaatttgttccggcatttctggaaattccggaagcaacccgtatggtgaccagtaaaattattaaggaaatcaacgactgcccgatggatcgtgtggttgaaatttggtttgaaggtccggatcagtggagcgcagccatggaaaaagccaaaagtattgaaccgccggaatgggcccaggaagaaacctttccttatctgaaaccgtatcagaatattatgggtgtttttgtggccgataatattagcagcgataatctgaataaccatcgcggttatattaccatgcgttaa

SEQ ID NO: 61 - DNA, 837 bp, Roseburia sp. OM02-15
ggtgaaattacctatcagccgctgcgcagtctgatctatgttgattgtgttaaagaagaataccgtcataaactgcagcattggctgtattattatcatgcaccggaaagcattgccaaatttgaaccgtgggtgaccaaatatgcattttataatgcactgccgaccccgccgcagggtgaacgttttggtacctatcgtatgcagctgaccgaacattattggatggtgaatccgatgagtcaggaaagcctggtgaatagcctgaatgaatattttccgattgatgccatgatttggcagggcaccctgccggaaaatgccaaagaagatggctttatggcagatggcgaagcagcacgcgccagcggtggtgacaatggctgtccgccgtttatttttgcatttctgccgattttctgggaacaggatctgaaaggcgccggccgtaccatggttgatggtccgaattatcgttggcagtttctggtgagttatccggaaggccataaagaagatggtgaccagtggatgatgaatgaagtgctgccggcctttgcagccatgccggaagtgacccgtatgatgaccagtaaagtgaaaaaagaaatcaacaactgccacatggatcgttgcgttgaaatgtggtttgatggtccggaagaatggtatcattgtgcagtggaaaatgcagcaagctttaaaaaaccggaatgggccgaatatgataattttccgtatctgcgcccgcagtttcagtttgcaagtctgtttctgaccgatattgcaaccagtgataatctgaaacagtatcgtggttatctgccgatgcgttaa

SEQ ID NO: 62 - DNA, 861 bp, Clostridium sp. SY8519
gcagattttgaattcaacccgatgcgcagtctgatctatattgattgtgttaaaaacgactaccgtcataaactgcagtattggctgtataaacatcatattccggatagtattgcaaaatttgaaccgtatgttagtaagtatgccttttatagcgcactgccggtgccggaagatggtgaagaatttggcacctataattatcagctgaccgaacattattggctgtgtaatccgctgaccgcagattttgcagtgaatagtctgcaggaaagttttaccccggatgttctgaaatggcagggtattattccggatattgatattaccccggaaatgatggccgccatgcagaatgccgatgatgcacgcgccagcgaaggcagcgaagcccctggttttgatctgccgccgtttatttttgcctttctgccgctgagttgggaatttcagaccaaaggtgaaaaactgaccattgaagatggtccgaattatcgctggcagtttctggttaaatatccggaagcagcagatgccgaacaggccgataaatggctgatggaagaagtgattccggcctttgcacagatggatgaagttaaacgtattctgagtagtaaagtgaaacaggaagttaatggttgcgtttattatcgtgttgttgaaatgtggtttgatagcccgagcgattggcatcgtgccgttaaagaaaaaaccggcgccattagtaaaccggaatgggcaagtaccgatagttttccgtatctgcgcccgaaacatgaaatggcaagcatttttctgagcgatattccgaccaccgataatctgctgcagtatcgtggttttattccgatgcgttaa

SEQ ID NO: 63 - DNA, 852 bp, Eubacterium sp
accaaaaacatgctgggttttgaaccgatgcgtagcctgatctatgtggatgtggccaaagaagattatcgcgttaaactggaaaattggctgtataaataccatattccggatagtattagccagtttggcccgtatgtgagtaaatatgccttttatcaggccctgccggtgccggaaggtggcgaacgttttggtgcaattaagatgcagctgaccgaacattattggctggccaatccgaataatattgcacagtttgatcatcataaggcactgaccgaatattttccgccggatgtgctgaaatggcagggtaatctgccggatgaagattttcagtgtgaaaatgtggaaggcgatgaagcccgtgccgttaaaggtgacgccagtaaaggtaccgtgccgtttatttttgcctttgttccggtttggtgggaagaagattataaaggtgccggtcgcaccattgaagatggcccgaattatcgttggcagatggttattaagtttccggatgatgccaaagataccggtgacaaatggctgcgcgatgaattcattccggcctttgttgaatgtgaagaaaccacccgtattctgagcagtaaagttattaaggaagccaatggttgtgaatttgatcgcgttgttgaaatgtggtttgaaggtccgagtgcatggaaagcagcagttggcaaagcagcaaaaaaggtgcgtcgtccggcctggtttgaaaccgaagattttccgtatctgacccagagctatggtattagcagcatttttctgagcgatattgcccgtagtgataatatgacccagtataatggttatgtgaccatgcgctaa

SEQ ID NO: 64 - DNA, 834 bp, Holophaga foetida
accgtggaattttgcccgatgcgcagcctgatctatgttggcctgagcaaagaaaaatatcgtcatctgctgcagaattggctgtatcgcattcatattccggatagcattagccagtttggcccgtatgttaccaaatatgccttttataatgccctgccggtgccgccggaaggtgaacgttttggcaccattaagatgcagctgaccgaacattattggcagtgcaatccgtttcagcaggcagtggccgttaaaacctttaaagaagtgtttccgattgatgttctgcgctggcagggtaatatgccggatgaaagcgatggcgccgttgaactggaaggcgatgaagcccgtagcgcaaaaggtggccagggcgcaaaaccgtttatttttgcatttgttccgctgtggtgggaagaagatattaagggcaaaatgcgcaccattgccgatggtccgaattatcgctggcagtttgtgatgaaatatccggaaggtgttagtcaggaacagggcgataaatggtttctggaagaagtggcaccggcctttgccgcaatgccggaagtgaatcgcattctgagcagtaaaattattcagaatgtgaatggttgcagctttaatcgtgtggttgaaatgtggtttgatggcccggatgaatggtatgccgcagcagttaccaaagccgcagcactgccgaaaccgacctgggcaaaacaggataaatttccgtttctggcaagtgattatgaatttgccagcgtgtttctgaccgattatgccaccagtgataatctgagccagtatcgtggctatattaccctgcgttaa

SEQ ID NO: 65 - DNA, 837 bp, Lactobacillus sp. 54-2
agctttgaattccgcccgatgcgtagcgttatctatgttgatgttattaaggaaaagtaccgcccgaaactgctgcattggctgtatggccatcatattcaggatagtattagcaaattcggcccgtatgtgaccaaatatgccttttataatgcctttccggttccgccggaaggtgaacgttttggtacccgtcgtatgcagatgaccgaacattattggctgattaatgaaatgaccccggaaatgaaaaataatgcaattaccgaatacatgccgaaagatgtgctgcgttggcagggtaatattccggataccgatgcagcagccgccggcaatgttgatggcgataccggccgcagcgcaggtggcgcaaatggttgtcagccgtttgtttttgcccatgtgccgattaattgggaagaagattataaaggtgacggccgtatggtggccgatggcccgaattatcgctggcagtttgtgattaagtatccggaaaatctgaccgaagcagccggtgaagcctggtttcatgatgaagtggttccgtattttaaaggtcgcccggaagttcatcgctttatgagtagccgcattatgcaggatgttgtgggttgtgaatttgaacgtctggttgaaatgtggtttgatagtgaagatgaatggtataccgcagccgtggccggcaccaccgcactgaaaaaaccgagctgggcccagcagaatcagtttccgtttctgaccccgcagtttaatattgttggcatgtttctgccggatattccgaccagtgataatctggcccagtatcgcggttatattaccatgcgttaa

SEQ ID NO: 66 - DNA, 849 bp, Butyrivibrio sp. AC2005
gcatacgaattcaatctgatgcgcagcctgatctatgtgggtctggcaaaagaagaatatcgtgcaaaactggaacattggctgtataatcatcatgttccggaaagtattagtaaattttctccgtatgtgaccaaatatgccttttatcaggcatatccgaccccgccggaaggtgaccgttttggtgcccgtaaaatgcagctgaccgaacattattggctggtgaatgaacatctgccggaaatggccaataatatttttcaggaattcatgccgatggatgttctgcgttggcagggctgtattccggatgttgatagcgccaccgttcatgataatgcagaaagtggtgacgccggccgcgccgttggcggtggtgactttccgccgtttatttttgcatttgttccgattaattgggaagaagattttaaaggtaaagagcgtaccccggtgaatggcccgaattttcgttggcagtttctgattcagtatccggaaggctgtagcaaagatgaaggtgaacgttggtttcatgaagaagttgtgccgtttctgaccggtagcgtgcgtgttaatcgtctggttagtagtaaagttaaagttaattacggcgccccgagtgcacagtttgatcgtctggttgaagtgtggtttgatggtccggaagaatggtatgatctgtgtgtgaatctggcaaaagataaactgaaaaaaccggaatgggcacagcaggatacctttccgtttctgaaaccgcagcataatattgccagtatgtttctgggtgaccgttgtaccagcgataatctgacccagtatcgcggttttattaccatgcgctaa

SEQ ID NO: 67 - DNA, 849 bp, Clostridioides difficile
gcagattttgaattcaagatcatgcgtagcctgatctatgttggcctggcaaaagaagaatatcgtccgaaactgatggattggctgtatcgtcatcatattccggatagtattagtacctttggcccgtattgcaccaaatatgcattttatcaggcctatccgaccccgaatgaaggcgaacgctttggtgcccgcaaaatgcagctgaccgaacattattggctggtggatgaacacatgccggaaatggcaaatcgtattatgaccgaatatatgccgatggatgtgctgcgctggcagggttgcattccggatgtggaaaataagcgtgtgcatgaaaatgtggaaagcggcgatgccggccgcgccgttggtggtgacaatggctgtccgccgtttatttttgcatttgttccgattaattgggaagaagattttcgcggtaaaggccgtaccgttcaggatggcccgaattatcgctggcagtttatgattaagtatccggatggtattagcaaagaagaaggtgaaaaatggttttatgacgaagttgtgccgtattttaccaattgctgttatgttaatcgctttgtgagtagcaaaattatgattaattacggcgcaaccgcatttgatcgtgtgagcgaactgtggtttgaaggtgaagaagaatggtataaagcagttgtggaagaaaccaaaagttttattaagaagccggaatgggcacaggaagaagaatttccgtatctgaaaccgcagtttaatattgccagcgtgtttctgggtgacattgccaccatggatgcctatagccagtatcgtggttatattccgatgcgttaa

SEQ ID NO: 68 - DNA, 849 bp, Eubacterium ramulus
gcagatttcaaattcgaaccaatgagaagtcttatctacgttgactgcgtaagcgaagactacagaccaaaacttcagagatggatttataaagtacatattccggacagcatctctcagtttgagccgtatgttaccaaatatgcattttatccgtccttcccgattccaccacagggtgatcgtttcggatacgcaagaatgcagctgacagagcatcactggttagtaagcgaccttgatcctcgtcttgagatcaaagcaatcgctgagacattcccgatggacgtacttgtatggcagggacagatcccggcagcagctcatacagacgctcagatcgattctgacggagatgcaggaaatgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 69 - protein, 276 aa, Fusibacter sp. 3D3
SYVPEFKPMRSFILNNVRSDNDRPKAFRWLYKQHVPESISQFRPYCTKYATYHALPLVEGAEQFGTYNWIMTEHYWNFNIFQDKGLGHSRGLAFTEAYPHDFMEITCQPPSNTLRQSGWQGSAEGYHPIVFCFAPIFWENDFKGSERLTDDGANFRWLFFLSYPEGVTVEEGDQWFLETFAKEAAEMPEVIRFLSSRVMDSPRINPFDRIVELWFDDSEAWHNAMVKNGHLFTKPAWSKVDQFPFFEAYKDITGVFIMDRPDSDHLAQWQGYITTR

SEQ ID NO: 70 - protein, 278 aa, Tepidanaerobacter acetatoxydans
DTEFKPMRSLIYVGVAKEEYRHKLKNWLYRYHIPDSISQFAPYVTKYAFYNALPVPPEGERFGAYNMQLTEHYWLINPMNPQLKIKAFTEFFPVDVLKWQGNIPDDADADSVNIEGDAARSSGGDNGMPPFIFAFIPFWWEEDFKGAGRTVEDGPNYRWQFLIQYPNGISLEEGDQWFYKKLVPAFKEMPEVNRFLTSRVIRDVNDCQFHRVIEMWFDGPEEWYRAAVEKGKSIERPHWATAEEFPYLKPKFQIASLFLTDIADSDNYSQYRGYITMR

SEQ ID NO: 71 - protein, 285 aa, Clostridium sp. JN500901
SIEFKPMRSLIYVDVVKEDYRHMLKHWLYYHHIPDSISQFGPYVTKYAFYPALPTPPDGERFGTHRMQLTEHYWLVNPLTPEFKNKTMTEYFPVDVLKWQGNVPDDNEVAILEGKETEKVESGDAARSTGGDNGMPPFIWAFVPISWEEDLKGEGRSMEDGPNYRWQFVMKYPDGVPMEEGDKWFYKEVIPKFQDSPDVTRILTSKIIQNINDCPYQRIVEMWFNCPSGWHRAAVEKAREIKKPSWAKYDEFPYLKPKFEISSLFLTDIAESDNYTQYRGFIPMR

SEQ ID NO: 72 - protein, 285 aa, Acetoanaerobium noterae
ADFEFNPMRSLIYVDVVKEDYRHMLKHWLYRHHIPDSISQFGPYVTKYAFYPALPTPPEGDRFGTIRMQLTEHYWTVNPFTSNFKNKAFTEFFPIDILKWQGNIPDDSKVAALEGEEVVKLEGDDARASGGDNGCPPFIFAFVPICWEEDFKGSQRTVEDGPNYRWQFVMKYPEGVTMKEGDKWFYEEMIPKFKDMPDVLRILSSKIIQQVNGCPYHRVVEMWFNCPSAWYRSAVEKAKEIKKPSWAKYDVFPYLKPKFEIASLFLTDIAESDNYSQYRGYITMR

SEQ ID NO: 73 - protein, 276 aa, Parasporobacterium paucivorans
GPEFQPMRSLIYVDCVKEEYRHRLQNWLYHYHVPDSISQFAPYVSKYCFYNALPTPPEGERFGTIRQQLTEHYWMVNPFTEHFKNKALTEYFPCDVLKWQGNIPDDDLEVNFEGDEARSKGGHNGCAPFIFAFLPLCWEEDFKGKERTIENGPNYRWNFVMKYPEGTSMEEGDKWFYEKMIPAFQEMPEVTRILTSKVIQSINGCAFHRVVEMWFDGPNAWYQAAVVKAKNIEKPSWASVDAFPYLQATFEFDGIFLTDYAASDNMSQYRGYLAMR

SEQ ID NO: 74 - protein, 277 aa, Lachnoclostridium sp.
GTEFDPMRSFIYVDVIKEEYRHKLQHWLYFHHIPESISQFEPYCTKYAFYNALPVPEGGERFGTARMQITEHSWLVNIMDPALKVKAFQEYMPIEALKWQGTVPETAGDVLMSGDDARCTGGNNGCMPFVFAFCPMWWEEDVKGAGRTLTEGANYRWNFALTYPEGISKEEGDKWLFEEVFPYFEANNCCTRILTSKVRQEINGCPMYRVVEMWFTGPTAWHHLAVETADKIKSPSWAKDGDVFPYLKSGFEIRGAFVTDYPTCDAYSQYRGYIPRR

SEQ ID NO: 75 - protein, 289 aa, Clostridium sp. SY8519
ADFKFEPMRSFIYVNCAKEDYRPKLQRWLYKTHVPDSIGQFEPYVTKYAFYPSFPTPPEGERFGYCSMQLTEHHWMVSDFDERLGIKAIAETFPPEVLLWQGNITEGMLEAMKKNGGAPGDDGGDSGNEARSTNMEGANPFIFCFAPYWWEDDIKGKGRTIEDGANYRWNVAIGYPEGVSAEEGDRWLFEEVIPVFEAAPECTRILHSKVKKEINGGSGMEHILEFWFENQSGWYQTAVEATKTLKKPAWAQQDQFPFLKPYHKFISVAVADFTMANNLVNYHGYIPRR

SEQ ID NO: 76 - protein, 267 aa, Roseburia sp.
RSLIYVDVAKEQYRVKLNHWLYAVHVPESISKFRPYVSQYAFYNALPVPPLGEQFGTVRMQLTEHYWQVNPMDVSMAVNVFTETFPTEVLIWQGNMPENAIALNYEGDEARSAKVENCNPFVFAFLPISWEEDLKGAGRTMADGPNYRWNFALHYPDGVSVETGDQWFYEKFVPAFLEIPEATRMVTSKIIKEINDCPMDRVVEIWFEGPDQWSAAMEKAKSIEPPEWAQEETFPYLKPYQNIMGVFVADNISSDNLNNHRGYITMR

SEQ ID NO: 77 - protein, 278 aa, Roseburia sp. OM02-15
GEITYQPLRSLIYVDCVKEEYRHKLQHWLYYYHAPESIAKFEPWVTKYAFYNALPTPPQGERFGTYRMQLTEHYWMVNPMSQESLVNSLNEYFPIDAMIWQGTLPENAKEDGFMADGEAARASGGDNGCPPFIFAFLPIFWEQDLKGAGRTMVDGPNYRWQFLVSYPEGHKEDGDQWMMNEVLPAFAAMPEVTRMMTSKVKKEINNCHMDRCVEMWFDGPEEWYHCAVENAASFKKPEWAEYDNFPYLRPQFQFASLFLTDIATSDNLKQYRGYLPMR

SEQ ID NO: 78 - protein, 286 aa, Clostridium sp. SY8519
ADFEFNPMRSLIYIDCVKNDYRHKLQYWLYKHHIPDSIAKFEPYVSKYAFYSALPVPEDGEEFGTYNYQLTEHYWLCNPLTADFAVNSLQESFTPDVLKWQGIIPDIDITPEMMAAMQNADDARASEGSEAPGFDLPPFIFAFLPLSWEFQTKGEKLTIEDGPNYRWQFLVKYPEAADAEQADKWLMEEVIPAFAQMDEVKRILSSKVKQEVNGCVYYRVVEMWFDSPSDWHRAVKEKTGAISKPEWASTDSFPYLRPKHEMASIFLSDIPTTDNLLQYRGFIPMR

SEQ ID NO: 79 - protein, 283 aa, Eubacterium sp
TKNMLGFEPMRSLIYVDVAKEDYRVKLENWLYKYHIPDSISQFGPYVSKYAFYQALPVPEGGERFGAIKMQLTEHYWLANPNNIAQFDHHKALTEYFPPDVLKWQGNLPDEDFQCENVEGDEARAVKGDASKGTVPFIFAFVPVWWEEDYKGAGRTIEDGPNYRWQMVIKFPDDAKDTGDKWLRDEFIPAFVECEETTRILSSKVIKEANGCEFDRVVEMWFEGPSAWKAAVGKAAKKVRRPAWFETEDFPYLTQSYGISSIFLSDIARSDNMTQYNGYVTMR

SEQ ID NO: 80 - protein, 277 aa, Holophaga foetida
TVEFCPMRSLIYVGLSKEKYRHLLQNWLYRIHIPDSISQFGPYVTKYAFYNALPVPPEGERFGTIKMQLTEHYWQCNPFQQAVAVKTFKEVFPIDVLRWQGNMPDESDGAVELEGDEARSAKGGQGAKPFIFAFVPLWWEEDIKGKMRTIADGPNYRWQFVMKYPEGVSQEQGDKWFLEEVAPAFAAMPEVNRILSSKIIQNVNGCSFNRVVEMWFDGPDEWYAAAVTKAAALPKPTWAKQDKFPFLASDYEFASVFLTDYATSDNLSQYRGYITLR

SEQ ID NO: 81 - protein, 278 aa, Lactobacillus sp. 54-2
SFEFRPMRSVIYVDVIKEKYRPKLLHWLYGHHIQDSISKFGPYVTKYAFYNAFPVPPEGERFGTRRMQMTEHYWLINEMTPEMKNNAITEYMPKDVLRWQGNIPDTDAAAAGNVDGDTGRSAGGANGCQPFVFAHVPINWEEDYKGDGRMVADGPNYRWQFVIKYPENLTEAAGEAWFHDEVVPYFKGRPEVHRFMSSRIMQDVVGCEFERLVEMWFDSEDEWYTAAVAGTTALKKPSWAQQNQFPFLTPQFNIVGMFLPDIPTSDNLAQYRGYITMR

SEQ ID NO: 82 - protein, 282 aa, Butyrivibrio sp. AC2005
AYEFNLMRSLIYVGLAKEEYRAKLEHWLYNHHVPESISKFSPYVTKYAFYQAYPTPPEGDRFGARKMQLTEHYWLVNEHLPEMANNIFQEFMPMDVLRWQGCIPDVDSATVHDNAESGDAGRAVGGGDFPPFIFAFVPINWEEDFKGKERTPVNGPNFRWQFLIQYPEGCSKDEGERWFHEEVVPFLTGSVRVNRLVSSKVKVNYGAPSAQFDRLVEVWFDGPEEWYDLCVNLAKDKLKKPEWAQQDTFPFLKPQHNIASMFLGDRCTSDNLTQYRGFITMR

SEQ ID NO: 83 - protein, 282 aa, Clostridioides difficile
ADFEFKIMRSLIYVGLAKEEYRPKLMDWLYRHHIPDSISTFGPYCTKYAFYQAYPTPNEGERFGARKMQLTEHYWLVDEHMPEMANRIMTEYMPMDVLRWQGCIPDVENKRVHENVESGDAGRAVGGDNGCPPFIFAFVPINWEEDFRGKGRTVQDGPNYRWQFMIKYPDGISKEEGEKWFYDEVVPYFTNCCYVNRFVSSKIMINYGATAFDRVSELWFEGEEEWYKAVVEETKSFIKKPEWAQEEEFPYLKPQFNIASVFLGDIATMDAYSQYRGYIPMR

SEQ ID NO: 84 - protein, 282 aa, Eubacterium ramulus
ADFKFEPMRSLIYVDCVSEDYRPKLQRWIYKVHIPDSISQFEPYVTKYAFYPSFPIPPQGDRFGYARMQLTEHHWLVSDLDPRLEIKAIAETFPMDVLVWQGQIPAAAHTDAQIDSDGDAGNAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 85 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaaatttgagccgtatgttaccaaatatgcattttatccgtccttcccgattccaccacagggtgatcgtttcggatacgcaagaatgcagctgacagagcatcactggttagtaagcgaacttgatcctcgtcttgagatcaatgcaatcgctgagacattcccgatggacgtacttgtatggcagggacagatcccggcagcagctcatacagacgctcagatcgattctgacggagatgcaggacgtgcagccggcaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 86 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctcagtttgagccgtatgttaccaaatatgcattttatccgtccttcccgattccaccacagggtgatcgtttcggatacgcaagaatgcagctgacagagcatcactggttagtaagcccgcttgatcctcgtcttgagatcaaagcaatcgctgagacattcccgatggacgtacttgtatggcagggacagatcccggcagcagctcatacagacgctcagatcgattctgacggagatgcaggacgtgcagccggcaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 87 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaattttgagccgtatgttaccaaatatgcattttatccgtccttcccgattccaccacagggtgatcgtttcggatacgcaagaatgcagctgacagagcatcactggttagtaagcgaacttgatcctcgtcttgagatcaaagcaatcgctgagacattcccgatggacgtacttgtatggcagggacagatcccggcagcagctcatacagacgctcagatcgattctgacggagatgcaggacgtgcagccggcaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 88 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctcagtttgagccgtatgttaccaaatatgcattttatccgtccttcccgattccaccacagggtgatcgtttcggatacgcaagaatgcagctgacagagcatcactggttagtaagcgaacttgatcctcgtcttgagatcaaagcaatcgctgagacattcccgatggacgtacttgtatggcagggacagatcccggcagcagctcatacagacgctcagatcgattctgacggagatgcaggacgtgcagccaaaaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 89 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaaatttgagccgtatgttaccaaatatgcattttatccgtccttcccgattccaccacagggtgatcgtttcggatacgcaagaatgcagctgacagagcatcactggttagtaagcccgcttgatcctcgtcttgagatcaatgcaatcgctgagacattcccgatggacgtacttgtatggcagggacagatcccggcagcagctcatacagacgctcagatcgattctgacggagatgcaggacgtgcagccaaaaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 90 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaattttgagccgtatgttaccaaatatgcattttatccgtccttcccgattccaccacagggtgatcgtttcggatacgcaagaatgcagctgacagagcatcactggttagtaagcgaacttgatcctcgtcttgagatccgtgcaatcgctgagacattcccgatggacgtacttgtatggcagggacagatcccggcagcagctcatacagacgctcagatcgattctgacggagatgcaggagatgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 91 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctacctttgagccgtatgttaccaaatatgcattttatccgtccttcccgattccaccacagggtgatcgtttcggatacgcaagaatgcagctgacagagcatcactggttagtaagcgaacttgatcctcgtcttgagatccgtgcaatcgctgagacattcccgatggacgtacttgtatggcagggacagatcccggcagcagctcatacagacgctcagatcgattctgacggagatgcaggagatgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 92 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaaatttgagccgtatgttaccaaatatgcattttatccgtccttcccgattccaccacagggtgatcgtttcggatacgcaagaatgcagctgacagagcatcactggttagtaagcccgcttgatcctcgtcttgagatcaatgcaatcgctgagacattcccgatggacgtacttgtatggcagggacagatcccggcagcagctcatacagacgctcagatcgattctgacggagatgcaggagaagcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 93 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctcagtttgagccgtatgttaccaaatatgcattttatccgtccttcccgattccaccacagggtgatcgtttcggatacgcaagaatgcagctgacagagcatcactggttagtaagcatgcttgatcctcgtcttgagatcaaagcaatcgctgagacattcccgatggacgtacttgtatggcagggacagatcccggcagcagctcatacagacgctcagatcgattctgacggagatgcaggaaatgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 94 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctcagtttgagccgtatgttaccaaatatgcattttatccgtccttcccgattccaccacagggtgatcgtttcggatacgcaagaatgcagctgacagagcatcactggttagtaagcattcttgatcctcgtcttgagatcaaagcaatcgctgagacattcccgatggacgtacttgtatggcagggacagatcccggcagcagctcatacagacgctcagatcgattctgacggagatgcaggagatgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 95 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctcagtttgagccgtatgttaccaaatatgcattttatccgtccttcccgattccaccacagggtgatcgtttcggatacgcaagaatgcagctgacagagcatcactggttagtaagcccgcttgatcctcgtcttgagatcaaagcaatcgctgagacattcccgatggacgtacttgtatggcagggacagatcccggcagcagctcatacagacgctcagatcgattctgacggagatgcaggagatgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 96 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaaatttgagccgtatgttaccaaatatgcattttatccgtccttcccgattccaccacagggtgatcgtttcggatacgcaagaatgcagctgacagagcatcactggttagtaagcccgcttgatcctcgtcttgagatcaatgcaatcgctgagacattcccgatggacgtacttgtatggcagggacagatcccggcagcagctcatacagacgctcagatcgattctgacggagatgcagcgcgtgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 97 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaaatttgagccgtatgttaccaaatatgcattttatccgtccttcccgattccaccacagggtgatcgtttcggatacgcaagaatgcagctgacagagcatcactggttagtaagcccgcttgatcctcgtcttgagatcaatgcaatcgctgagacattcccgatggacgtacttgtatggcagggacagatcccggcagcagctcatacagacgctcagatcgattctgacggagatgcagatgatgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 98 - DNA, 852 bp, Eubacterium ramulus
atggcagatttcaaattcgaaccaatgagaagtcttatctacgttgactgcgtaagcgaagactacagaccaaaacttcagagatggatttataaagtacatattccggacagcatctctaaatttgagccgtatgttaccaaatatgcattttatccgtccttcccgattccaccacagggtgatcgtttcggatacgcaagaatgcagctgacagagcatcactggttagtaagcgaacttgatcctcgtcttgagatcaatgcaatcgctgagacattcccgatggacgtacttgtatggcagggacagatcccggcagcagctcatacagacgctcagatcgattctgacggagatgcacatcgtgcagcccgtaaatccaacaatgcagaaggaaatccatttatctttgcattccttccgatgtggtgggagaaagacctgaaaggaaaaggacgtacgatcgaggacggcgcaaactatcgtttcaatatgactatcggtttcccagaaggcgtagacaaagcagagggagagaaatggttatttgagaaagtagttccgattcttcaggcagctccggagtgtacacgtgtacttgcaagtgccgtaaagaaagacatcaacggatgcgtaatggattgggtacttgaaatctggtttgagaatcagtccggatggtacaaagtaatggtagatgacatgaaagcacttgaaaaaccgtcatgggctcagcaggatgctttcccgttcctgaaaccataccacaatgtttgcagtgcagcagttgctgattatacaccaagcaacaaccttgcaaattatcgtggatacatcaccatgagataa

SEQ ID NO: 99 - DNA, 49 bp, Artificial Sequence
  Forward Primer
ccaacaatgcagaaggaaatccagcgatctttgcattccttccgatgtg

SEQ ID NO: 100 - DNA, 49 bp, Artificial Sequence
  Reverse Primer
ccaacaatgcagaaggaaatccaccgatctttgcattccttccgatgtg

SEQ ID NO: 101 - DNA, 49 bp, Artificial Sequence; identical to SEQ ID NO: 100
  Forward Primer

SEQ ID NO: 102 - DNA, 49 bp, Artificial Sequence
  Reverse Primer
cacatcggaaggaatgcaaagatcggtggatttccttctgcattgttgg

SEQ ID NO: 103 - DNA, 41 bp, Artificial Sequence
  Forward Primer
agatgcaggaaatgcagccggcaaatccaacaatgcagaag

SEQ ID NO: 104 - DNA, 41 bp, Artificial Sequence
  Reverse Primer
cttctgcattgttggatttgccggctgcatttcctgcatct

SEQ ID NO: 105 - DNA, 45 bp, Artificial Sequence
  Forward Primer
ggagatgcaggaaatgcagccgtgaaatccaacaatgcagaagga

SEQ ID NO: 106 - DNA, 45 bp, Artificial Sequence
  Reverse Primer
tccttctgcattgttggatttcacggctgcatttcctgcatctcc

SEQ ID NO: 107 - DNA, 33 bp, Artificial Sequence
  Forward Primer
tgacggagatgcaggagatgcagcccgtaaatc

SEQ ID NO: 108 - DNA, 33 bp, Artificial Sequence
  Reverse Primer
gatttacgggctgcatctcctgcatctccgtca

SEQ ID NO: 109 - DNA, 35 bp, Artificial Sequence
  Forward Primer
ctgacggagatgcaggacgtgcagcccgtaaatcc

SEQ ID NO: 110 - DNA, 35 bp, Artificial Sequence
  Reverse Primer
ggatttacgggctgcacgtcctgcatctccgtcag

SEQ ID NO: 111 - DNA, 35 bp, Artificial Sequence
  Forward Primer
ctgacggagatgcaggagaagcagcccgtaaatcc

SEQ ID NO: 112 - DNA, 35 bp, Artificial Sequence
  Reverse Primer
ggatttacgggctgcttctcctgcatctccgtcag

SEQ ID NO: 113 - DNA, 43 bp, Artificial Sequence
  Forward Primer
gattctgacggagatgcaggaggcgcagcccgtaaatccaaca

SEQ ID NO: 114 - DNA, 43 bp, Artificial Sequence
  Reverse Primer
tgttggatttacgggctgcgcctcctgcatctccgtcagaatc

SEQ ID NO: 115 - DNA, 33 bp, Artificial Sequence
  Forward Primer
tgacggagatgcaggacatgcagcccgtaaatc

SEQ ID NO: 116 - DNA, 33 bp, Artificial Sequence
  Reverse Primer
gatttacgggctgcatgtcctgcatctccgtca

SEQ ID NO: 117 - DNA, 43 bp, Artificial Sequence
  Forward Primer
gattctgacggagatgcaggagcggcagcccgtaaatccaaca

SEQ ID NO: 118 - DNA, 43 bp, Artificial Sequence
  Reverse Primer
tgttggatttacgggctgccgctcctgcatctccgtcagaatc

SEQ ID NO: 119 - DNA, 35 bp, Artificial Sequence
  Forward Primer
ctgacggagatgcaggaagcgcagcccgtaaatcc

SEQ ID NO: 120 - DNA, 35 bp, Artificial Sequence
  Reverse Primer
ggatttacgggctgcgcttcctgcatctccgtcag

SEQ ID NO: 121 - DNA, 35 bp, Artificial Sequence
  Forward Primer
tcctcgtcttgagatcaatgcaatcgctgagacat

SEQ ID NO: 122 - DNA, 35 bp, Artificial Sequence
  Reverse Primer
atgtctcagcgattgcattgatctcaagacgagga

SEQ ID NO: 123 - DNA, 45 bp, Artificial Sequence
  Forward Primer
cttgatcctcgtcttgagatccgtgcaatcgctgagacattcccg

SEQ ID NO: 124 - DNA, 45 bp, Artificial Sequence
  Reverse Primer
cgggaatgtctcagcgattgcacggatctcaagacgaggatcaag

SEQ ID NO: 125 - DNA, 45 bp, Artificial Sequence
  Reverse Primer
gagcatcactggttagtaagcccgcttgatcctcgtcttgagatc

SEQ ID NO: 126 - DNA, 45 bp, Artificial Sequence
  Reverse Primer
gatctcaagacgaggatcaagcgggcttactaaccagtgatgctc

SEQ ID NO: 127 - DNA, 45 bp, Artificial Sequence
  Forward Primer
gagcatcactggttagtaagcatgcttgatcctcgtcttgagatc

SEQ ID NO: 128 - DNA, 45 bp, Artificial Sequence
  Reverse Primer
gatctcaagacgaggatcaagcatgcttactaaccagtgatgctc

SEQ ID NO: 129 - DNA, 35 bp, Artificial Sequence
  Forward Primer
tcactggttagtaagcgaacttgatcctcgtcttg

SEQ ID NO: 130 - DNA, 35 bp, Artificial Sequence
  Reverse Primer
caagacgaggatcaagttcgcttactaaccagtga

SEQ ID NO: 131 - DNA, 50 bp, Artificial Sequence
  Forward Primer
acagcatctctcagtttgagccgggcgttaccaaatatgcattttatccg

SEQ ID NO: 132 - DNA, 50 bp, Artificial Sequence
  Reverse Primer
cggataaaatgcatatttggtaacgcccggctcaaactgagagatgctgt

SEQ ID NO: 133 - DNA, 50 bp, Artificial Sequence
  Forward Primer
acagcatctctcagtttgagccggcggttaccaaatatgcattttatccg

SEQ ID NO: 134 - DNA, 50 bp, Artificial Sequence
  Reverse Primer
cggataaaatgcatatttggtaaccgccggctcaaactgagagatgctgt

SEQ ID NO: 135 - DNA, 44 bp, Artificial Sequence
  Forward Primer
gcatctctcagtttgagccggatgttaccaaatatgcattttat

SEQ ID NO: 136 - DNA, 44 bp, Artificial Sequence
  Reverse Primer
ataaaatgcatatttggtaacatccggctcaaactgagagatgc

SEQ ID NO: 137 - DNA, 45 bp, Artificial Sequence
  Forward Primer
catattccggacagcatctctaaatttgagccgtatgttaccaaa

SEQ ID NO: 138 - DNA, 45 bp, Artificial Sequence
  Reverse Primer
tttggtaacatacggctcaaatttagagatgctgtccggaatatg

SEQ ID NO: 139 - DNA, 45 bp, Artificial Sequence
  Forward Primer
catattccggacagcatctctaattttgagccgtatgttaccaaa

SEQ ID NO: 140 - DNA, 45 bp, Artificial Sequence
  Reverse Primer
tttggtaacatacggctcaaaattagagatgctgtccggaatatg

SEQ ID NO: 141 - DNA, 51 bp, Artificial Sequence
  Forward Primer
gtacatattccggacagcatctctacctttgagccgtatgttaccaaatat

SEQ ID NO: 142 - DNA, 51 bp, Artificial Sequence
  Reverse Primer
atatttggtaacatacggctcaaaggtagagatgctgtccggaatatgtac

SEQ ID NO: 143 - DNA, 53 bp, Artificial Sequence
  Forward Primer
agtacatattccggacagcatctctatttttgagccgtatgttaccaaatatg

SEQ ID NO: 144 - DNA, 53 bp, Artificial Sequence
  Reverse Primer
catatttggtaacatacggctcaaaaatagagatgctgtccggaatatgtact

SEQ ID NO: 145 - protein, 283 aa, Eubacterium ramulus
MADFKFEPMRSLIYVDCVSEDYRPKLQRWIYKVHIPDSISKFEPYVTKYAFYPSFPIPPQGDRFGYARMQLTEHHWLVSELDPRLEINAIAETFPMDVLVWQGQIPAAAHTDAQIDSDGDAGRAAGKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 146 - protein, 283 aa, Eubacterium ramulus
MADFKFEPMRSLIYVDCVSEDYRPKLQRWIYKVHIPDSISQFEPYVTKYAFYPSFPIPPQGDRFGYARMQLTEHHWLVSPLDPRLEIKAIAETFPMDVLVWQGQIPAAAHTDAQIDSDGDAGRAAGKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 147 - protein, 283 aa, Eubacterium ramulus
MADFKFEPMRSLIYVDCVSEDYRPKLQRWIYKVHIPDSISNFEPYVTKYAFYPSFPIPPQGDRFGYARMQLTEHHWLVSELDPRLEIKAIAETFPMDVLVWQGQIPAAAHTDAQIDSDGDAGRAAGKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 148 - protein, 283 aa, Eubacterium ramulus
MADFKFEPMRSLIYVDCVSEDYRPKLQRWIYKVHIPDSISQFEPYVTKYAFYPSFPIPPQGDRFGYARMQLTEHHWLVSELDPRLEIKAIAETFPMDVLVWQGQIPAAAHTDAQIDSDGDAGRAAKKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 149 - protein, 283 aa, Eubacterium ramulus
MADFKFEPMRSLIYVDCVSEDYRPKLQRWIYKVHIPDSISKFEPYVTKYAFYPSFPIPPQGDRFGYARMQLTEHHWLVSPLDPRLEINAIAETFPMDVLVWQGQIPAAAHTDAQIDSDGDAGRAAKKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 150 - protein, 283 aa, Eubacterium ramulus
MADFKFEPMRSLIYVDCVSEDYRPKLQRWIYKVHIPDSISNFEPYVTKYAFYPSFPIPPQGDRFGYARMQLTEHHWLVSELDPRLEIRAIAETFPMDVLVWQGQIPAAAHTDAQIDSDGDAGDAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 151 - protein, 283 aa, Eubacterium ramulus
MADFKFEPMRSLIYVDCVSEDYRPKLQRWIYKVHIPDSISTFEPYVTKYAFYPSFPIPPQGDRFGYARMQLTEHHWLVSELDPRLEIRAIAETFPMDVLVWQGQIPAAAHTDAQIDSDGDAGDAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 152 - protein, 283 aa, Eubacterium ramulus
MADFKFEPMRSLIYVDCVSEDYRPKLQRWIYKVHIPDSISKFEPYVTKYAFYPSFPIPPQGDRFGYARMQLTEHHWLVSPLDPRLEINAIAETFPMDVLVWQGQIPAAAHTDAQIDSDGDAGEAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 153 - protein, 283 aa, Eubacterium ramulus
MADFKFEPMRSLIYVDCVSEDYRPKLQRWIYKVHIPDSISQFEPYVTKYAFYPSFPIPPQGDRFGYARMQLTEHHWLVSMLDPRLEIKAIAETFPMDVLVWQGQIPAAAHTDAQIDSDGDAGNAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 154 - protein, 283 aa, Eubacterium ramulus
MADFKFEPMRSLIYVDCVSEDYRPKLQRWIYKVHIPDSISQFEPYVTKYAFYPSFPIPPQGDRFGYARMQLTEHHWLVSILDPRLEIKAIAETFPMDVLVWQGQIPAAAHTDAQIDSDGDAGDAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 155 - protein, 283 aa, Eubacterium ramulus
MADFKFEPMRSLIYVDCVSEDYRPKLQRWIYKVHIPDSISQFEPYVTKYAFYPSFPIPPQGDRFGYARMQLTEHHWLVSPLDPRLEIKAIAETFPMDVLVWQGQIPAAAHTDAQIDSDGDAGDAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 156 - protein, 283 aa, Eubacterium ramulus
MADFKFEPMRSLIYVDCVSEDYRPKLQRWIYKVHIPDSISKFEPYVTKYAFYPSFPIPPQGDRFGYARMQLTEHHWLVSPLDPRLEINAIAETFPMDVLVWQGQIPAAAHTDAQIDSDGDAARAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 157 - protein, 283 aa, Eubacterium ramulus
MADFKFEPMRSLIYVDCVSEDYRPKLQRWIYKVHIPDSISKFEPYVTKYAFYPSFPIPPQGDRFGYARMQLTEHHWLVSPLDPRLEINAIAETFPMDVLVWQGQIPAAAHTDAQIDSDGDADDAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 158 - protein, 283 aa, Eubacterium ramulus
MADFKFEPMRSLIYVDCVSEDYRPKLQRWIYKVHIPDSISKFEPYVTKYAFYPSFPIPPQGDRFGYARMQLTEHHWLVSELDPRLEINAIAETFPMDVLVWQGQIPAAAHTDAQIDSDGDAHRAARKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR

SEQ ID NO: 159 - DNA, 1094 bp, Arabidopsis thaliana
gcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttgtagtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 160 - protein, 365 aa, Arabidopsis thaliana
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFVVSDFYDKYSKFLEFVLPHIREGKITYVEDVADGLEKAPEALVGLFHGKNVGKQVVVVARE

SEQ ID NO: 161 - DNA, 1098 bp, Artificial Sequence
  AtDBR1 variant V285Q
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttcaggtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 162 - DNA, 1098 bp, Artificial Sequence
  AtDBR1 variant V285T
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttaccgtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 163 - DNA, 1098 bp, Artificial Sequence
  AtDBR1 variant V285D
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttgacgtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 164 - DNA, 1098 bp, Artificial Sequence
  AtDBR1 variant V285L
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttttggtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 165 - DNA, 1098 bp, Artificial Sequence
  AtDBR1 variant Y81F
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatttggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttgtagtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 166 - DNA, 1098 bp, Artificial Sequence
  AtDBR1 variant Y276A
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatcgctaagcgtaatcgtattcaaggctttgtagtcagcgacttctacgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 167 - DNA, 1098 bp, Artificial Sequence
  AtDBR1 variant Y290A
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttgtagtcagcgacttcgctgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 168 - DNA, 1098 bp, Artificial Sequence
  AtDBR1 variant Y290F
atgggcagcagccatcatcatcatcatcacagcagcggcctggtgccgcgcggcagccatatgacagccaccaacaaacaggtcatcttaaaagactacgtatctggctttcctaccgaaagtgactttgacttcacgaccacgaccgtggagttgcgtgttcctgaaggtaccaacagtgtcctggttaaaaacctgtacctgtcgtgcgatccgtacatgcgtattcgcatgggcaagccggatccgtcgacggcggccctggcacaagcgtacacccccggtcaacctatccaaggatatggcgtatctcgcatcatcgaatccggacacccggattacaagaaaggcgatctgttgtggggcattgtggcgtgggaagaatattcggtaatcaccccaatgactcatgcgcatttcaaaattcagcacaccgacgtccctctgagctattacaccggtctgctgggcatgccgggtatgaccgcatatgccggtttctatgaagtttgtagcccgaaagaaggggagacagtgtacgtgagcgcagcatccggcgccgtgggacagctggtgggccagctcgcaaaaatgatgggttgttatgtcgtcggtagcgccggcagcaaggaaaaagttgatctgctgaagacgaaattcggcttcgatgacgcctttaactataaagaagaatcagatctgaccgcggcgctgaagcgttgtttcccgaacggtattgatatttacttcgaaaacgttgggggtaaaatgctggatgcggtgttggtcaacatgaacatgcacggccgcattgctgtgtgcggcatgatttctcaatataatttggaaaaccaggaaggtgttcataatctgagcaatattatctataagcgtaatcgtattcaaggctttgtagtcagcgacttctttgataaatattcgaagtttttggaatttgtactgccgcacattcgtgaaggcaaaatcacctacgtcgaggatgtagcggatggactggaaaaagccccagaggcgcttgtcggtctgttccatggtaaaaatgtagggaaacaggttgttgttgtggcacgtgaataa

SEQ ID NO: 169 - protein, 365 aa, Artificial Sequence
  AtDBR1 variant V285Q
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFQVSDFYDKYSKFLEFVLPHIREGKITYVEDVADGLEKAPEALVGLFHGKNVGKQVVVVARE

SEQ ID NO: 170 - protein, 365 aa, Artificial Sequence
  AtDBR1 variant V285T
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFTVSDFYDKYSKFLEFVLPHIREGKITYVEDVADGLEKAPEALVGLFHGKNVGKQVVVVARE

SEQ ID NO: 171 - protein, 365 aa, Artificial Sequence
  AtDBR1 variant V285D
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFDVSDFYDKYSKFLEFVLPHIREGKITYVEDVADGLEKAPEALVGLFHGKNVGKQVVVVARE

SEQ ID NO: 172 - protein, 365 aa, Artificial Sequence
  AtDBR1 variant V285L
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFLVSDFYDKYSKFLEFVLPHIREGKITYVEDVADGLEKAPEALVGLFHGKNVGKQVVVVARE

SEQ ID NO: 173 - protein, 365 aa, Artificial Sequence
  AtDBR1 variant Y81F
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGFGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFVVSDFYDKYSKFLEFVLPHIREGKITYVEDVADGLEKAPEALVGLFHGKNVGKQVVVVARE

SEQ ID NO: 174 - protein, 365 aa, Artificial Sequence
  AtDBR1 variant Y276A
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIAKRNRIQGFVVSDFYDKYSKFLEFVLPHIREGKITYVEDVADGLEKAPEALVGLFHGKNVGKQVVVVARE

SEQ ID NO: 175 - protein, 365 aa, Artificial Sequence
  AtDBR1 variant Y290A
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFVVSDFADKYSKFLEFVLPHIREGKITYVEDVADGLEKAPEALVGLFHGKNVGKQVVVVARE

SEQ ID NO: 176 - protein, 365 aa, Artificial Sequence
  AtDBR1 variant Y290F
MGSSHHHHHHSSGLVPRGSHMTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRNRIQGFVVSDFFDKYSKFLEFVLPHIREGKITYVEDVADGLEKAPEALVGLFHGKNVGKQVVVVARE

SEQ ID NO: 177 - DNA, 27 bp, Artificial Sequence
  Reverse Primer
atacgattacgcttatagataatattg

SEQ ID NO: 178 - DNA, 25 bp, Artificial Sequence
  Forward Primer
tcaaggctttcaggtcagcgacttc

SEQ ID NO: 179 - DNA, 25 bp, Artificial Sequence
  Forward primer
tcaaggctttaccgtcagcgacttc

SEQ ID NO: 180 - DNA, 25 bp, Artificial Sequence
  Forward primer
tcaaggctttgacgtcagcgacttc

SEQ ID NO: 181 - DNA, 25 bp, Artificial Sequence
  Forward primer
tcaaggctttttggtcagcgacttc

SEQ ID NO: 182 - DNA, 18 bp, Artificial Sequence
  Reverse Primer
ggttgaccgggggtgtac

SEQ ID NO: 183 - DNA, 25 bp, Artificial Sequence
  Forward primer
tatccaaggatttggcgtatctcgc

SEQ ID NO: 184 - DNA, 20 bp, Artificial Sequence
  Reverse Primer
ctcagattatgaacaccttc

SEQ ID NO: 185 - DNA, 32 bp, Artificial Sequence
  Forward primer
caatattatcgctaagcgtaatcgtattcaag

SEQ ID NO: 186 - DNA, 19 bp, Artificial Sequence
  Forward primer
actacaaagccttgaatac

SEQ ID NO: 187 - DNA, 34 bp, Artificial Sequence
  Forward primer
cagcgacttcgctgataaatattcgaagtttttg

SEQ ID NO: 188 - DNA, 28 bp, Artificial Sequence
  Forward primer
cagcgacttctttgataaatattcgaag

SEQ ID NO: 189 - protein, 345 aa, Artificial Sequence
  Consensus sequence ene reductase
  misc_feature (81)..(81): Xaa can be any naturally occurring amino acid
  misc_feature (276)..(276): Xaa can be any naturally occurring amino acid
  misc_feature (285)..(285): Xaa can be any naturally occurring amino acid
  misc_feature (290)..(290): Xaa can be any naturally occurring amino acid
MTATNKQVILKDYVSGFPTESDFDFTTTTVELRVPEGTNSVLVKNLYLSCDPYMRIRMGKPDPSTAALAQAYTPGQPIQGXGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIXKRNRIQGFXVSDFXDKYSKFLEFVLPHIREGKITYVEDVADGLEKAPEALVGLFHGKNVGKQVVVVARE

SEQ ID NO: 190 - protein, 283 aa, Artificial Sequence
  Consensus sequence chalcone isomerase
  misc_feature (41)..(41): Xaa can be any naturally occurring amino acid
  misc_feature (80)..(80): Xaa can be any naturally occurring amino acid
  misc_feature (88)..(88): Xaa can be any naturally occurring amino acid
  misc_feature (122)..(123): Xaa can be any naturally occurring amino acid
  misc_feature (126)..(126): Xaa can be any naturally occurring amino acid
MADFKFEPMRSLIYVDCVSEDYRPKLQRWIYKVHIPDSISXFEPYVTKYAFYPSFPIPPQGDRFGYARMQLTEHHWLVSXLDPRLEIXAIAETFPMDVLVWQGQIPAAAHTDAQIDSDGDAXXAAXKSNNAEGNPFIFAFLPMWWEKDLKGKGRTIEDGANYRFNMTIGFPEGVDKAEGEKWLFEKVVPILQAAPECTRVLASAVKKDINGCVMDWVLEIWFENQSGWYKVMVDDMKALEKPSWAQQDAFPFLKPYHNVCSAAVADYTPSNNLANYRGYITMR